SEQUENCE LISTING (1 sequence)

SEQ ID NO: 1 - DNA, 19 bp, synthetic construct
  source 1..19: mol_type = other DNA
atgacggatcagccggcaa

(19) United States
(12) Patent Application Publication
(10) Pub. No.: US 2026/0100241 A1
(43) Pub. Date: Apr. 9, 2026

(54) PREDICTING EFFECTS OF GENE REGULATORY SEQUENCES ON ENDOPHENOTYPES USING MACHINE LEARNING

(71) Applicant: Inari Agriculture Technology, Inc., Cambridge, MA (US)

(72) Inventors: Ross Everett ALTMAN, Boston, MA (US); Karl Anton Grothe KREMLING, Somerville, MA (US)

(73) Assignee: Inari Agriculture Technology, Inc., Cambridge, MA (US)

(21) Appl. No.: 18/877,673

(22) PCT Filed: Jun. 23, 2023
(86) PCT No.: PCT/US2023/069025

§ 371 Date: Dec. 20, 2024

Related U.S. Application Data
(60) Provisional application No. 63/355,513, filed on Jun. 24, 2022.

(51) Int. Cl. G16B 5/20; G06N 20/00; G16B 20/50; G16B 40/20

(52) U.S. Cl. CPC G16B 5/20; G06N 20/00; G16B 20/50; G16B 40/20

(57) ABSTRACT

A method for generating a gene regulatory sequence with a desired endophenotype profile includes obtaining a plurality of gene regulatory sequences and inputting the plurality of gene regulatory sequences into a machine-learning model trained to obtain a plurality of effect predictions corresponding to a plurality of endophenotypes. The method further includes selecting one or more desired endophenotypes based on the plurality of endophenotypes and selecting a gene regulatory sequence in accordance with the one or more desired endophenotypes.

### CROSS-REFERENCE TO RELATED APPLICATIONS

This application claims priority to U.S. Provisional Patent Application No. 63/355,513, filed on Jun. 24, 2022, the entirety of which is incorporated herein by reference.

### REFERENCE TO AN ELECTRONIC SEQUENCE LISTING

The contents of the electronic sequence listing (165362000740SEQLIST.xml; Size: 1,980 bytes; and Date of Creation: Jun. 23, 2023) is herein incorporated by reference in its entirety.

### TECHNICAL FIELD

This application relates generally to gene endophenotypes, and, more particularly, to predicting gene endophenotypes based on mutations in gene regulatory sequences and factors.

### BACKGROUND

A thorough investigation of the organization, function, and evolution of plant genes can be paramount to ascertaining and manipulating certain complex plant biological processes allowing development of plants with improved traits. In many instances, ascertaining and manipulating such complex plant biological processes may often be performed by determining and manipulating, for example, the genes and regulatory mechanisms controlling these biological processes. Often genes are knocked in, knocked out, or mutated to produce a desired phenotype. However, while it may be a very time- and/or space-inefficient process to wait for a phenotype to develop in a mature plant, a variety of intermediate endophenotype biomarkers may also present at smaller scales during the processing of genetic information which serve as effective indicators of the ultimate phenotype. Such regulators may be classified in terms of their structure as cis regulatory sequences and trans regulatory factors and represent a powerful way to modulate the amount or timing of endophenotypes of one or multiple genes.

For example, the cis regulatory sequences may include linear nucleotide fragments of non-coding DNA, in which the cis regulatory sequences may be located directly adjacent to or in the transcribed DNA strand including promoters, enhancers, silencers, insulators, and so forth. Similarly, the trans regulatory factors may include, for example, certain regulatory proteins that may interact with the cis regulatory sequences and/or other proteins to form active complexes. Therefore, understanding such cis and trans regulatory elements in plants has the possibility of allowing for rational engineering of plants to produce plants with beneficial traits.

### BRIEF SUMMARY

In one aspect, the present disclosure provides a method of modifying an endophenotype in a plant, the method comprising, by one or more computing devices: obtaining a plurality of gene regulatory sequences; inputting the plurality of gene regulatory sequences into a machine-learning model trained to obtain a plurality of effect predictions corresponding to a plurality of endophenotypes; selecting one or more desired endophenotypes based on the plurality of endophenotypes; selecting a gene regulatory sequence in accordance with the one or more desired endophenotypes, and introducing the selected gene regulatory sequence into the plant, thereby modifying the endophenotype of the plant.

In another aspect, the present disclosure provides a method for generating a gene regulatory sequence with a desired endophenotype profile, the method comprising, by one or more computing devices: obtaining a plurality of gene regulatory sequences; inputting the plurality of gene regulatory sequences into a machine-learning model trained to obtain a plurality of effect predictions corresponding to a plurality of endophenotypes; selecting one or more desired endophenotypes based on the plurality of endophenotypes; and selecting a gene regulatory sequence in accordance with the one or more desired endophenotypes. In some embodiments, the method further comprises introducing the selected gene regulatory sequence into the plant, thereby modifying the endophenotype of the plant. In some embodiments, selecting the gene regulatory sequence comprises selecting a gene regulatory sequence in accordance with a desired endophenotype level. In some embodiments, the desired endophenotype level comprises a desired messenger RNA (mRNA) expression level. In some embodiments, the one or more computing devices are associated with a genome editing platform, the genome editing platform configured to generate the gene regulatory sequence with the desired endophenotype profile. In some embodiments, obtaining the plurality of gene regulatory sequences comprises obtaining a plurality of gene promoter regulatory sequences, a plurality of gene terminator regulatory sequences, a plurality of gene enhancer regulatory sequences, a plurality of gene repressor regulatory sequences, or a plurality of transcription factor binding sites. In some embodiments, the machine-learning model comprises one or more sequence encoder models. In some embodiments, the machine-learning model is trained by: pre-training a randomly-initialized sequence encoder model utilizing a self-supervised prediction of the one or more gene regulatory sequences; and fine-tuning the pre-trained sequence encoder model utilizing a self-supervised prediction of a plurality of gene regulatory sequences extracted from a targeted taxonomic unit. In some embodiments, the machine-learning model is trained further by: utilizing a variant effect predictor model with inputs generated by the sequence encoder model to: 1) further fine-tune the weights of the sequence encoder model and 2) generate effect predictions corresponding to a plurality of candidate endophenotypes of interest. In other embodiments, the machine-learning model is trained further by: computing a loss value based on a comparison of the effect predictions and an endophenotype measurement; and training the variant effect predictor model based on a backpropagation of the computed loss value. In some embodiments, the method further comprises utilizing the variant effect predictor model to predict a particular endophenotype measurement observed from one or more cell-based assays or one or more plant-based assays. In some embodiments, the machine-learning model comprises one or more sequence space-sampling algorithms. In some embodiments, the method further comprises, subsequent to obtaining the plurality of gene regulatory sequences: inputting a plurality of seed gene regulatory sequences into the one or more sequence space-sampling algorithms; and obtaining the plurality of effect predictions by: 1) computationally sampling the space of gene regulatory sequences, and 2) inputting the plurality of sampled gene regulatory sequences into the one or more trained machine-learning models to obtain the plurality of effect predictions corresponding to the plurality of endophenotypes. In some embodiments, the one or more sequence space-sampling algorithms comprise one or more generative adversarial networks (GANs), one or more variational autoencoders (VAEs), or one or more Markov chain Monte Carlo (MCMC) sampling algorithms. In some embodiments, obtaining the plurality of effect predictions corresponding to the plurality of endophenotypes comprises iteratively providing as feedback a plurality of sampled gene regulatory sequences as seed sequences for the one or more sequence space-sampling algorithms until the one or more desired endophenotypes are produced. In some embodiments, obtaining the plurality of gene regulatory sequences comprises obtaining a plurality of synthetic gene regulatory sequences. In some embodiments, the selected gene regulatory sequence is operably linked to an exogenous or endogenous transcript, and is provided in a vector for expressing the exogenous or endogenous transcript. In some embodiments, the method further comprises generating a guide comprising the gene regulatory sequence or a portion thereof. In some embodiments, the method further comprises generating a guide, where generating the guide comprises generating one or more guide RNAs (gRNAs). In some embodiments, the guide RNA and/or donor template nucleic acid is configured to introduce a selected modified gene regulatory sequence into one or more plants. In some embodiments, the machine-learning model was trained utilizing data representing information related to RNAseq, microarrays, ribosome profiling, single cell RNASeq, and/or proteome abundance. In some embodiments, the one or more desired endophenotypes comprises a tissue-specific gene endophenotype, a temporally-controlled gene endophenotype, or a change in gene endophenotype in response to a stimulus. In some embodiments, the method further comprises transforming a seed with the gene regulatory sequence. In some embodiments, the method further comprises growing a plant comprising a modified gene regulatory sequence from the transformed seed. In some embodiments, the method further comprises introducing the selected gene regulatory sequence into a plant. In another aspect, the present disclosure provides a plant comprising a modified gene regulatory sequence generated by the method of any of the previous embodiments.

In another aspect, the present disclosure provides a system including one or more computing devices, comprising: one or more non-transitory computer-readable storage media including instructions; and one or more processors coupled to the one or more storage media, the one or more processors configured to execute the instructions to: obtain a plurality of gene regulatory sequences; input the plurality of gene regulatory sequences into a machine-learning model trained to obtain a plurality of effect predictions corresponding to a plurality of endophenotypes; select one or more desired endophenotypes based on the plurality of endophenotypes; and select a gene regulatory sequence in accordance with the one or more desired endophenotypes. In some embodiments, the instructions to select the gene regulatory sequence further comprise instructions to select a gene regulatory sequence in accordance with a desired endophenotype level. In some embodiments, the one or more desired endophenotypes comprises a desired messenger RNA (mRNA) expression level. In some embodiments, the one or more computing devices are associated with a genome editing platform, the genome editing platform configured to generate the gene regulatory sequence with the desired endophenotype profile. In some embodiments, the instructions to obtain the plurality of gene regulatory sequences further comprise instructions to obtain a plurality of gene promoter regulatory sequences, a plurality of gene terminator regulatory sequences, a plurality of gene enhancer regulatory sequences, a plurality of gene repressor regulatory sequences, or a plurality of transcription factor binding sites. In some embodiments, the machine-learning model comprises one or more sequence encoder models. In some embodiments, the machine-learning model is trained by: pre-training a randomly-initialized sequence encoder model utilizing a self-supervised prediction of the one or more gene regulatory sequences; and fine-tuning the pre-trained sequence encoder model utilizing a self-supervised prediction of a plurality of gene regulatory sequences extracted from a targeted taxonomic unit. In some embodiments, the machine-learning model is trained further by: utilizing a variant effect predictor model with inputs generated by the sequence encoder model to: 1) further fine-tune the weights of the sequence encoder model and 2) generate effect predictions corresponding to a plurality of candidate endophenotypes of interest. In some embodiments, the machine-learning model is trained further by: computing a loss value based on a comparison of the effect predictions and an endophenotype measurement; and training the variant effect predictor model based on a backpropagation of the computed loss value. In some embodiments, the instructions further comprise instructions to utilize the variant effect predictor model to predict a particular endophenotype measurement observed from one or more cell-based assays or one or more plant-based assays. In some embodiments, the machine-learning model comprises one or more sequence space-sampling algorithms. In some embodiments, the instructions further comprise instructions to: subsequent to obtaining the plurality of gene regulatory sequences: input a plurality of seed gene regulatory sequences into the one or more sequence space-sampling algorithms; and obtain the plurality of effect predictions by: 1) computationally sampling the space of gene regulatory sequences, and 2) inputting the plurality of sampled gene regulatory sequences into the one or more trained machine-learning models to obtain the plurality of effect predictions corresponding to the plurality of endophenotypes. In some embodiments, the one or more sequence space-sampling algorithms comprise one or more generative adversarial networks (GANs), one or more variational autoencoders (VAEs), or one or more Markov chain Monte Carlo (MCMC) sampling algorithms. In some embodiments, the instructions to obtain the plurality of effect predictions corresponding to the plurality of endophenotypes further comprise instructions to iteratively provide as feedback a plurality of sampled gene regulatory sequences as seed sequences for the one or more sequence space-sampling algorithms until the one or more desired endophenotypes are produced. In some embodiments, the instructions to obtain the plurality of gene regulatory sequences further comprise instructions to obtain a plurality of synthetic gene regulatory sequences. In some embodiments, the selected gene regulatory sequence is operably linked to an exogenous or endogenous transcript, and is provided in a vector for expressing an exogenous or endogenous transcript. In some embodiments, the instructions further comprise instructions to generate a donor template nucleic acid comprising the gene regulatory sequence or a portion thereof. In some embodiments, the instructions further comprise instructions to generate one or more guide RNAs (gRNAs) targeting a genomic location to promote introduction of the gene regulatory sequence. In some embodiments, the guide RNA and/or donor template nucleic acid is configured to introduce a selected modified gene regulatory sequence into one or more plants. In some embodiments, the machine-learning model was trained utilizing data representing information related to RNAseq, microarrays, ribosome profiling, single cell RNASeq, and/or proteome abundance. In some embodiments, the one or more desired endophenotypes comprises a tissue-specific gene endophenotype, a temporally-controlled gene endophenotype, or a change in gene endophenotype in response to a stimulus. In some embodiments, the instructions further comprise instructions to transform a plant with the gene regulatory sequence.

In another aspect, the present disclosure provides a non-transitory computer-readable medium comprising instructions that, when executed by one or more processors of one or more computing devices, cause the one or more processors to: obtain a plurality of gene regulatory sequences; input the plurality of gene regulatory sequences into a machine-learning model trained to obtain a plurality of effect predictions corresponding to a plurality of endophenotypes; select one or more desired endophenotypes based on the plurality of endophenotypes; and select a gene regulatory sequence in accordance with the one or more desired endophenotypes. In some embodiments, the instructions to select the gene regulatory sequence further comprise instructions to select a gene regulatory sequence in accordance with a desired endophenotype level. In some embodiments, the desired endophenotype level comprises a desired messenger RNA (mRNA) expression level. In some embodiments, the one or more computing devices are associated with a genome editing platform, the genome editing platform configured to generate the gene regulatory sequence with the desired endophenotype profile. In some embodiments, the instructions to obtain the plurality of gene regulatory sequences further comprise instructions to obtain a plurality of gene promoter regulatory sequences, a plurality of gene terminator regulatory sequences, a plurality of gene enhancer regulatory sequences, a plurality of gene repressor regulatory sequences, or a plurality of transcription factor binding sites. In some embodiments, the machine-learning model comprises one or more sequence encoder models. In some embodiments, the machine-learning model is trained by: pre-training a randomly-initialized sequence encoder model utilizing a self-supervised prediction of the one or more gene regulatory sequences; and fine-tuning the pre-trained sequence encoder model utilizing a self-supervised prediction of a plurality of gene regulatory sequences extracted from a targeted taxonomic unit. In some embodiments, the machine-learning model is trained further by: utilizing a variant effect predictor model with inputs generated by the sequence encoder model to: 1) further fine-tune the weights of the sequence encoder model and 2) generate effect predictions corresponding to a plurality of candidate endophenotypes of interest. In some embodiments, the machine-learning model is trained further by: computing a loss value based on a comparison of the effect predictions and an endophenotype measurement; and training the variant effect predictor model based on a backpropagation of the computed loss value. In some embodiments, the instructions further comprise instructions to utilize the variant effect predictor model to predict a particular endophenotype measurement observed from one or more cell-based assays or one or more plant-based assays. In some embodiments, the machine-learning model comprises one or more sequence space-sampling algorithms. In some embodiments, the instructions further comprise instructions to: subsequent to obtaining the plurality of gene regulatory sequences: input a plurality of seed gene regulatory sequences into the one or more sequence space-sampling algorithms; and obtain the plurality of effect predictions by: 1) computationally sampling the space of gene regulatory sequences, and 2) inputting the plurality of sampled gene regulatory sequences into the one or more trained machine-learning models to obtain the plurality of effect predictions corresponding to the plurality of endophenotypes. In some embodiments, the instructions further comprise instructions to input the plurality of seed gene regulatory sequences into one or more generative adversarial networks (GANs), one or more variational autoencoders (VAEs), or one or more Markov chain Monte Carlo (MCMC) sampling algorithms. In some embodiments, the instructions to obtain the plurality of effect predictions corresponding to the plurality of endophenotypes further comprise instructions to iteratively provide as feedback a plurality of sampled gene regulatory sequences as seed sequences for the one or more sequence space-sampling algorithms until the one or more desired endophenotypes are produced. In some embodiments, the instructions to obtain the plurality of gene regulatory sequences further comprise instructions to obtain a plurality of synthetic gene regulatory sequences. In some embodiments, the gene regulatory sequence is utilized in a vector for expressing an exogenous or endogenous transcript. In some embodiments, the instructions further comprise instructions to generate a guide comprising the gene regulatory sequence. In some embodiments, the instructions to generate the guide further comprise instructions to generate one or more guide RNAs (gRNAs). In some embodiments, the guide is configured to produce a desired modified gene regulatory sequence in one or more plants. In some embodiments, the machine-learning model was trained utilizing data representing information related to RNAseq, microarrays, ribosome profiling, single cell RNASeq, and/or proteome abundance. In some embodiments, the one or more desired endophenotypes comprises a tissue-specific gene endophenotype, a temporally-controlled gene endophenotype, or a change in gene endophenotype in response to a stimulus. In some embodiments, the instructions further comprise instructions to transform a plant with the gene regulatory sequence.

In another aspect, the present disclosure provides a method for predicting the effect of a mutated gene regulatory sequence, the method comprising, by one or more computing devices: inputting a plurality of gene regulatory sequences to a first trained machine-learning model, the plurality of gene regulatory sequences comprising one or more mutated gene regulatory sequences; utilizing the first trained machine-learning model to generate a first set of gene-level endophenotype profiles based on the plurality of gene regulatory sequences, comprising cis regulatory effects of the one or more mutated gene regulatory sequences; inputting the first set of gene-level endophenotype profiles to a second trained machine-learning model; and utilizing the second trained machine-learning model to generate a second set of gene-level endophenotype profiles based on the first set of gene-level endophenotype profiles, wherein generating the second set of gene-level endophenotype profiles comprises predicting one or more updated gene-level endophenotype profiles based on the plurality of gene regulatory sequences including the trans regulatory effects of the one or more mutated gene regulatory sequences. In some embodiments, the one or more computing devices are associated with a genome editing platform, and wherein the genome editing platform is configured to predict the effect of the one or more mutated gene regulatory sequences on all genes in the genome or pathway due to both cis and trans regulatory effects. In some embodiments, the method further comprises providing as feedback the predicted second set of gene-level endophenotype profiles to the second trained machine-learning model. In some embodiments, providing as feedback the predicted second set of gene-level endophenotype profiles to the second trained machine-learning model comprises refining the prediction of the second set of gene-level endophenotype profiles in accordance with a predetermined evaluation metric. In some embodiments, the first trained machine-learning model comprises one or more sequence encoder models including language-based models adapted from natural language processing (NLP) and one or more variant effect predictor models including classification or regression models. In some embodiments, the method further comprises: training the first trained machine-learning model by: pre-training a randomly-initialized language model utilizing a self-supervised prediction of one or more gene regulatory sequences extracted from a wide variety of species; and fine-tuning the pre-trained language model utilizing a self-supervised prediction of a plurality of gene regulatory sequences extracted from a targeted species. In some embodiments, training the first machine-learning model further comprises: training a regression or classification model with input features generated by the fine-tuned language model to generate effect predictions corresponding to a plurality of candidate endophenotypes of interest. In some embodiments, the method further comprises utilizing the regression or classification model to predict a particular endophenotype measurement observed from one or more cell-based assays or one or more plant-based assays. In some embodiments, the method further comprises further comprising: observing the particular endophenotype measurement from the one or more cell-based assays or one or more plant-based assays; and training the regression or classification model by: computing a loss value based on a comparison of the effect predictions and the endophenotype measurement; and training the regression or classification model based on a backpropagation of the computed loss value. In some embodiments, the second trained machine-learning model comprises one or more graph neural networks (GNNs). In some embodiments, the method further comprises training the second machine-learning model by: aggregating a dataset of endophenotype profiles of various genotypes corresponding to a target organism or pathway; and selecting one or more random pairs of genotypes from the dataset of endophenotype profiles of genotypes for which each pair represents an unmodified and modified organism respectively. In some embodiments, training the second machine-learning model further comprises: initializing the one or more GNNs by randomly partitioning nodes of graphs comprising the one or more GNNs into a first set of nodes corresponding to a first genotype of the one or more random pairs of genotypes and a second set of nodes corresponding to a second genotype of the one or more random pairs of genotypes. In some embodiments, training the second machine-learning model further comprises: training the one or more GNNs to predict the endophenotypes corresponding to first set of nodes for the second genotype given both the endophenotypes corresponding to the first set of nodes for the first genotype and the endophenotypes corresponding to the second set of nodes for the second genotype. In some embodiments, the second set of gene-level endophenotype profiles is predicted for a modified genotype of one or more plant seeds. In some embodiments, the first trained machine-learning model and the second trained machine-learning model were trained utilizing data representing information related to RNAseq, microarrays, ribosome profiling, single cell RNASeq, and/or proteome abundance. In some embodiments, the first set of gene-level endophenotype profiles comprises a tissue-specific gene endophenotype, a temporally-controlled gene endophenotype, or a change in gene endophenotype in response to a stimulus. In some embodiments, the method further comprises transforming a seed based on the one or more predicted gene-level endophenotype profiles. In some embodiments, the method further comprises growing a plant comprising predicted gene-level endophenotype profiles from the transformed seed. In some embodiments, the method further comprises introducing a mutant gene regulatory sequence to a plant based on the one or more predicted gene-level endophenotype profiles. In another aspect, the present disclosure also provides a plant comprising a mutated gene regulatory sequence and/or predicted gene-level endophenotype profiles generated by the method of any one of the previous embodiments.

In another aspect, the present disclosure provides a system including one or more computing devices, comprising: one or more non-transitory computer-readable storage media including instructions; and one or more processors coupled to the one or more storage media, the one or more processors configured to execute the instructions to: input a plurality of gene regulatory sequences to a first trained machine-learning model, the plurality of gene regulatory sequences including one or more mutated gene regulatory sequences; utilize the first trained machine-learning model to generate a first set of gene-level endophenotype profiles based on the plurality of gene regulatory sequences, including the cis regulatory effects of the one or more mutated gene regulatory sequences; input the first set of gene-level endophenotype profiles to a second trained machine-learning model; and utilize the second trained machine-learning model to generate a second set of gene-level endophenotype profiles based on the first set of gene-level endophenotype profiles, wherein generating the second set of gene-level endophenotype profiles comprises predicting one or more updated gene-level endophenotype profiles based on the plurality of gene regulatory sequences including the trans regulatory effects of the one or more mutated gene regulatory sequences. In some embodiments, the one or more computing devices are associated with a genome editing platform, and wherein the genome editing platform is configured to assess the effect of the one or more mutated gene regulatory sequences on all genes in the genome or pathway due to both cis and trans regulatory effects. In some embodiments, the instructions further comprise instructions to provide as feedback the predicted second set of gene-level endophenotype profiles to the second trained machine-learning model. In some embodiments, the instructions to provide as feedback the predicted second set of gene-level endophenotype profiles to the second trained machine-learning model further comprise instructions to refine the prediction of the second set of gene-level endophenotype profiles in accordance with a predetermined evaluation metric. In some embodiments, the first trained machine-learning model comprises one or more sequence encoder models including language-based models adapted from natural language processing (NLP) and one or more variant effect predictor models including classification or regression models. In some embodiments, the instructions further comprise instructions to: train the first trained machine-learning model by: pre-training a randomly-initialized language model utilizing a self-supervised prediction of one or more gene regulatory sequences extracted from a wide variety of species; and fine-tuning the pre-trained language model utilizing a self-supervised prediction of a plurality of gene regulatory sequences extracted from a targeted species. In some embodiments, training the first machine-learning model further comprises: training a regression or classification model with input features generated by the fine-tuned language model to generate effect predictions corresponding to a plurality of candidate endophenotypes of interest. In some embodiments, the instructions further comprise instructions to utilize the regression or classification model to predict a particular endophenotype measurement observed from one or more cell-based assays or one or more plant-based assays. In some embodiments, the instructions further comprise instructions to: obtain the particular endophenotype measurement from the one or more cell-based assays or one or more plant-based assays; and train the regression or classification model by: computing a loss value based on a comparison of the effect predictions and the endophenotype measurement; and training the regression or classification model based on a backpropagation of the computed loss value. In some embodiments, the second trained machine-learning model comprises one or more graph neural networks (GNNs). In some embodiments, the instructions further comprise instructions to: train the second machine-learning model by: aggregating a dataset of endophenotype profiles of various genotypes corresponding to a target organism or pathway; and selecting one or more random pairs of genotypes from the dataset of endophenotype profiles of genotypes for which each pair represents an unmodified and modified organism respectively. In some embodiments, training the second machine-learning model further comprises: initializing the one or more GNNs by randomly partitioning nodes of graphs comprising the one or more GNNs into a first set of nodes corresponding to a first genotype of the one or more random pairs of genotypes and a second set of nodes corresponding to a second genotype of the one or more random pairs of genotypes. In some embodiments, training the second machine-learning model further comprises: training the one or more GNNs to predict the endophenotypes corresponding to first set of nodes for the second genotype given both the endophenotypes corresponding to the first set of nodes for the first genotype and the endophenotypes corresponding to the second set of nodes for the second genotype. In some embodiments, the second set of gene-level endophenotype profiles is predicted for a modified genotype of one or more plant seeds. In some embodiments, the first trained machine-learning model and the second trained machine-learning model were trained utilizing data representing information related to RNAseq, microarrays, ribosome profiling, single cell RNASeq, and/or proteome abundance. In some embodiments, the first set of gene-level endophenotype profiles comprises a tissue-specific gene endophenotype, a temporally-controlled gene endophenotype, or a change in gene endophenotype in response to a stimulus. In some embodiments, the instructions further comprise instructions to transform a plant based on the one or more predicted gene-level endophenotype profiles.

In another aspect, the present disclosure provides a non-transitory computer-readable medium comprising instructions that, when executed by one or more processors of one or more computing devices, cause the one or more processors to: input a plurality of gene regulatory sequences to a first trained machine-learning model, the plurality of gene regulatory sequences including one or more mutated gene regulatory sequences; utilize the first trained machine-learning model to generate a first set of gene-level endophenotype profiles based on the plurality of gene regulatory sequences, including the cis regulatory effects of the one or more mutated gene regulatory sequences; input the first set of gene-level endophenotype profiles to a second trained machine-learning model; and utilize the second trained machine-learning model to generate a second set of gene-level endophenotype profiles based on the first set of gene-level endophenotype profiles, wherein generating the second set of gene-level endophenotype profiles comprises predicting one or more updated gene-level endophenotype profiles based on the plurality of gene regulatory sequences including the trans regulatory effects of the one or more mutated gene regulatory sequences. In some embodiments, the one or more computing devices are associated with a genome editing platform, and wherein the genome editing platform is configured to predict the effect of the one or more mutated gene regulatory sequences on all genes in the genome or pathway due to both cis and trans regulatory effects. In some embodiments, the instructions further comprise instructions to provide as feedback the predicted second set of gene-level endophenotype profiles to the second trained machine-learning model. In some embodiments, the instructions to provide as feedback the predicted second set of gene-level endophenotype profiles to the second trained machine-learning model further comprise instructions to refine the prediction of the second set of gene-level endophenotype profiles in accordance with a predetermined evaluation metric. In some embodiments, the first trained machine-learning model comprises one or more sequence encoder models including language-based models adapted from natural language processing (NLP) and one or more variant effect predictor models including classification or regression models. In some embodiments, the instructions further comprise instructions to: train the first trained machine-learning model by: pre-training a randomly-initialized language model utilizing a self-supervised prediction of one or more gene regulatory sequences extracted from a wide variety of species; and fine-tuning the pre-trained language model utilizing a self-supervised prediction of a plurality of gene regulatory sequences extracted from a targeted species. In some embodiments, training the first machine-learning model further comprises: training a regression or classification model with input features generated by the fine-tuned language model to generate effect predictions corresponding to a plurality of candidate endophenotypes of interest. In some embodiments, the instructions further comprise instructions to utilize the regression or classification model to predict a particular endophenotype measurement observed from one or more cell-based assays or one or more plant-based assays. In some embodiments, the instructions further comprise instructions to: train the regression or classification model by: computing a loss value based on a comparison of the effect predictions and the endophenotype measurement; and training the regression or classification model based on a backpropagation of the computed loss value. In some embodiments, the second trained machine-learning model comprises one or more graph neural networks (GNNs). In some embodiments, the instructions further comprise instructions to: train the second machine-learning model by: aggregating a dataset of endophenotype profiles of various genotypes corresponding to a target organism or pathway; and selecting one or more random pairs of genotypes from the dataset of endophenotype profiles of genotypes for which each pair represents an unmodified and modified organism respectively. In some embodiments, training the second machine-learning model further comprises: initializing the one or more GNNs by randomly partitioning nodes of graphs comprising the one or more GNNs into a first set of nodes corresponding to a first genotype of the one or more random pairs of genotypes and a second set of nodes corresponding to a second genotype of the one or more random pairs of genotypes. In some embodiments, training the second machine-learning model further comprises: training the one or more GNNs to predict the endophenotypes corresponding to first set of nodes for the second genotype given both the endophenotypes corresponding to the first set of nodes for the first genotype and the endophenotypes corresponding to the second set of nodes for the second genotype. In some embodiments, the second set of gene-level endophenotype profiles is predicted for a modified genotype of one or more plant seeds. In some embodiments, the first trained machine-learning model and the second trained machine-learning model were trained utilizing data representing information related to RNAseq, microarrays, ribosome profiling, single cell RNASeq, and/or proteome abundance. In some embodiments, the first set of gene-level endophenotype profiles comprises a tissue-specific gene endophenotype, a temporally-controlled gene endophenotype, or a change in gene endophenotype in response to a stimulus. In some embodiments, the instructions further comprise instructions to transform a plant based on the one or more predicted gene-level endophenotype profiles.

In another aspect, the present disclosure provides a method of regulating two or more genes in a plant, the method comprising, a) by one or more computing devices: i) obtaining one or more endophenotype profiles corresponding to a genotype; ii) partitioning the one or more endophenotype profiles into a first set of endophenotypes and a second set of endophenotypes; iii) receiving an input to modify the first set of endophenotypes to a desired level; and iv) inputting the modified first set of endophenotypes and unmodified second set of endophenotypes into a trained machine-learning model to obtain a prediction of an updated second set of endophenotypes, wherein the updated second set of endophenotypes represents an updated version of the second set of endophenotypes after interacting with the modified first set of endophenotypes; and b) modifying an endophenotype level of one or more predicted interacting partner genes by modifying the first set of endophenotypes. In some embodiments, receiving an input to modify the first set of endophenotypes to a desired level comprises receiving one or more modified genotypes predicted to modify the first set of endophenotypes to the desired level. In some embodiments, modifying an endophenotype level of one or more predicted interacting partner genes by modifying the first set of endophenotypes comprises introducing the one or more modified genotypes into the plant. In some embodiments, the method further comprises, after step iv): v) comparing the prediction of the updated second set of endophenotypes to a desired level. In some embodiments, the method further comprises: vi) if the prediction of the updated second set of endophenotypes does not reach a desired level, return to step iii), receiving an input comprising an altered set of one or more modified genotypes predicted to modify the first set of endophenotypes to the desired level.

In one aspect, the present disclosure provides a method for predicting endophenotypes of interacting partner genes, the method comprising, by one or more computing devices: obtaining one or more endophenotype profiles corresponding to a genotype; partitioning the one or more endophenotype profiles into a first set of endophenotypes and a second set of endophenotypes; receiving an input to modify the first set of endophenotypes to a desired level; and inputting the modified first set of endophenotypes and unmodified second set of endophenotypes into a trained machine-learning model to obtain a prediction of an updated second set of endophenotypes, wherein the updated second set of endophenotypes represents an updated version of the second set of endophenotypes after interacting with the modified subset of the first set of endophenotypes. In some embodiments, receiving an input to modify the first set of endophenotypes to a desired level comprises receiving one or more modified genotypes predicted to modify the first set of endophenotypes to the desired level. In some embodiments, the method further comprises modifying an endophenotype level of one or more predicted interacting partner genes by modifying the first set of endophenotypes. In some embodiments, the one or more computing devices are associated with a genome editing platform, and wherein the genome editing platform is configured to assess updates to the second set of endophenotypes as a result of trans regulatory effects. In some embodiments, obtaining the one or more endophenotype profiles comprises obtaining one or more endophenotype profiles corresponding to a target genotype. In some embodiments, inputting the first set of endophenotypes into the trained machine-learning model comprises inputting node representation vectors to a graph neural network (GNN). In some embodiments, the method further comprises providing as feedback the updated second set of endophenotypes to the trained machine-learning model in place of the original second set of endophenotypes in order to refine the prediction of the updated second set of endophenotype levels in accordance with a predetermined evaluation metric. In some embodiments, the trained machine-learning model comprises one or more graph neural networks (GNNs). In some embodiments, nodes of graphs comprising the one or more GNNs represent genes associated with the target organism or pathway. In some embodiments, edges of the graphs comprising the one or more GNNs represent predictions of interactions in trans between genes associated with the target organism or pathway. In some embodiments, the graphs comprising the one or more GNNs further comprise one or more known gene co-expression relationships, one or more known protein-to-protein interactions, one or more gene ontology relationships, or a combination thereof. In some embodiments, the method further comprises training the machine-learning model by: aggregating a dataset of endophenotype profiles corresponding to various genotypes comprising a target organism or pathway; and selecting one or more random pairs of genotypes from the dataset of endophenotype profiles of genotypes for which each pair represents an unmodified and modified organism respectively. In some embodiments, training the machine-learning model further comprises: initializing one or more graph neural networks (GNNs) by randomly partitioning nodes of graphs comprising the one or more GNNs into a first set of nodes corresponding to a first genotype of the one or more random pairs of genotypes and a second set of nodes corresponding to a second genotype of the one or more random pairs of genotypes. In some embodiments, training the machine-learning model further comprises: training the one or more GNNs to predict the endophenotypes corresponding to the first set of nodes for the second genotype given both the endophenotypes corresponding to the first set of nodes for the first genotype and the endophenotypes corresponding to the second set of nodes for the second genotype. In some embodiments, obtaining the one or more endophenotype profiles comprises accessing an aggregate of a plurality of gene interaction data to be utilized to construct one or more gene regulatory network graphs. In some embodiments, the plurality of gene interaction data comprises one or more known gene co-expression relationships, one or more known protein-to-protein interactions, one or more gene ontology relationships, or a combination thereof. In some embodiments, the one or more predicted interacting partner genes in the genome comprises one or more predicted interacting partner genes in a modified genotype of one or more plant seeds. In some embodiments, the machine-learning model was trained utilizing data representing information related to RNAseq, microarrays, ribosome profiling, single cell RNASeq, and/or proteome abundance. In some embodiments, the endophenotype comprises a tissue-specific gene endophenotype, a temporally-controlled gene endophenotype, or a change in gene endophenotype in response to a stimulus. In some embodiments, the method further comprises providing genome editing molecules to a seed to introduce the one or more modified genotypes to the plant based on the one or more predicted endophenotype profiles. In some embodiments, the method further comprises growing a plant from the transformed seed. In some embodiments, the method further comprises providing genome editing molecules to a plant to introduce the one or more modified genotypes to the plant based on the one or more predicted endophenotype profiles. In some embodiments, the genome editing molecules comprise an endonuclease and one or more guide RNAs. In some embodiments, the genome editing molecules further comprise a donor template nucleic acid comprising the sequence of the one or more modified genotypes. In one aspect, the present disclosure also provides a plant comprising predicted endophenotype profiles generated by the method of any one of the previous embodiments.

In another aspect, the present disclosure provides a system including one or more computing devices, comprising: one or more non-transitory computer-readable storage media including instructions; and one or more processors coupled to the one or more storage media, the one or more processors configured to execute the instructions to: obtain one or more endophenotype profiles corresponding to a genotype; partition the one or more endophenotype profiles into a first set of endophenotypes and a second set of endophenotypes; receive an input to modify the first set of endophenotypes to a desired level; and input the modified first set of endophenotypes and unmodified second set of endophenotypes into a trained machine-learning model to obtain a prediction of an updated second set of endophenotypes, wherein the updated second set of endophenotypes represents an updated version of the second set of endophenotypes after interacting with the modified subset of the first set of endophenotypes. In some embodiments, the instructions to receive an input to modify the first set of endophenotypes to a desired level further comprise instructions to receive one or more modified genotypes predicted to modify the first set of endophenotypes to the desired level. In some embodiments, the one or more computing devices are associated with a genome editing platform, and wherein the genome editing platform is configured to assess updates to the second set of endophenotypes as a result of trans regulatory effects. In some embodiments, the instructions to obtain the one or more endophenotype profiles further comprise instructions to obtain one or more endophenotype profiles corresponding to a target genotype. In some embodiments, the instructions to input the first set of endophenotypes into the trained machine-learning model further comprise instructions to input node representation vectors to a graph neural network (GNN). In some embodiments, the instructions further comprise instructions to provide as feedback the updated second set of endophenotypes to the trained machine-learning model in place of the original second set of endophenotypes in order to refine the prediction of the updated second set of endophenotype levels in accordance with a predetermined evaluation metric. In some embodiments, the trained machine-learning model comprises one or more graph neural networks (GNNs). In some embodiments, nodes of graphs comprising the one or more GNNs represent genes associated with the target organism or pathway. In some embodiments, edges of the graphs comprising the one or more GNNs represent predictions of interactions in trans between genes associated with the target organism or pathway. In some embodiments, the graphs comprising the one or more GNNs further comprise one or more known gene co-expression relationships, one or more known protein-to-protein interactions, one or more gene ontology relationships, or a combination thereof. In some embodiments, the instructions further comprise instructions to: train the machine-learning model by: aggregating a dataset of endophenotype profiles corresponding to various genotypes comprising a target organism or pathway; and selecting one or more random pairs of genotypes from the dataset of endophenotype profiles of genotypes for which each pair represents an unmodified and modified organism respectively. In some embodiments, the instructions to train the machine-learning model further comprise instructions to: initialize one or more graph neural networks (GNNs) by randomly partitioning nodes of graphs comprising the one or more GNNs into a first set of nodes corresponding to a first genotype of the one or more random pairs of genotypes and a second set of nodes corresponding to a second genotype of the one or more random pairs of genotypes. In some embodiments, the instructions to train the machine-learning model further comprise instructions to: train the one or more GNNs to predict the endophenotypes corresponding to the first set of nodes for the second genotype given both the endophenotypes corresponding to the first set of nodes for the first genotype and the endophenotypes corresponding to the second set of nodes for the second genotype. In some embodiments, the instructions to obtain the one or more endophenotype profiles further comprise instructions to access an aggregate of a plurality of gene interaction data to be utilized to construct one or more gene regulatory network graphs. In some embodiments, the plurality of gene interaction data comprises one or more known gene co-expression relationships, one or more known protein-to-protein interactions, one or more gene ontology relationships, or a combination thereof. In some embodiments, the one or more predicted interacting partner genes in the genome comprises one or more predicted interacting partner genes in a modified genotype of one or more plant seeds. In some embodiments, the machine-learning model was trained utilizing data representing information related to RNAseq, microarrays, ribosome profiling, single cell RNASeq, and/or proteome abundance. In some embodiments, the endophenotype comprises a tissue-specific gene endophenotype, a temporally-controlled gene endophenotype, or a change in gene endophenotype in response to a stimulus. In some embodiments, the genome editing platform is further configured to introduce the one or more modified genotypes to a plant based on the one or more predicted endophenotype profiles.

In another aspect, the present disclosure provides a non-transitory computer-readable medium comprising instructions that, when executed by one or more processors of one or more computing devices, cause the one or more processors to: obtain one or more endophenotype profiles corresponding to a genotype; partition the one or more endophenotype profiles into a first set of endophenotypes and a second set of endophenotypes; receive an input to modify the first set of endophenotypes to a desired level; and input the modified first set of endophenotypes and unmodified second set of endophenotypes into a trained machine-learning model to obtain a prediction of an updated second set of endophenotypes, wherein the updated second set of endophenotypes represents an updated version of the second set of endophenotypes after interacting with the modified subset of the first set of endophenotypes. In some embodiments, the instructions to receive an input to modify the first set of endophenotypes to a desired level further comprise receiving one or more modified genotypes predicted to modify the first set of endophenotypes to the desired level. In some embodiments, the one or more computing devices are associated with a genome editing platform, and wherein the genome editing platform is configured to predict updates to the second set of endophenotypes as a result of trans regulatory effects. In some embodiments, the instructions to obtain the one or more endophenotype profiles further comprise instructions to obtain one or more endophenotype profiles corresponding to a target genotype. In some embodiments, the instructions to input the first set of endophenotypes into the trained machine-learning model further comprise instructions to input node representation vectors to a graph neural network (GNN). In some embodiments, the instructions further comprise instructions to provide as feedback the updated second set of endophenotypes to the trained machine-learning model in place of the original second set of endophenotypes in order to refine the prediction of the updated second set of endophenotype levels in accordance with a predetermined evaluation metric. In some embodiments, the trained machine-learning model comprises one or more graph neural networks (GNNs). In some embodiments, nodes of graphs comprising the one or more GNNs represent genes associated with the target organism or pathway. In some embodiments, edges of the graphs comprising the one or more GNNs represent predictions of interactions in trans between genes associated with the target organism or pathway. In some embodiments, the graphs comprising the one or more GNNs further comprise one or more known gene co-expression relationships, one or more known protein-to-protein interactions, one or more gene ontology relationships, or a combination thereof. In some embodiments, the instructions further comprise instructions to: train the machine-learning model by: aggregating a dataset of endophenotype profiles corresponding to various genotypes comprising a target organism or pathway; and selecting one or more random pairs of genotypes from the dataset of endophenotype profiles of genotypes for which each pair represents an unmodified and modified organism respectively. In some embodiments, the instructions to train the machine-learning model further comprise instructions to: initialize one or more graph neural networks (GNNs) by randomly partitioning nodes of graphs comprising the one or more GNNs into a first set of nodes corresponding to a first genotype of the one or more random pairs of genotypes and a second set of nodes corresponding to a second genotype of the one or more random pairs of genotypes. In some embodiments, the instructions to train the machine-learning model further comprise instructions to: train the one or more GNNs to predict the endophenotypes corresponding to the first set of nodes for the second genotype given both the endophenotypes corresponding to the first set of nodes for the first genotype and the endophenotypes corresponding to the second set of nodes for the second genotype. In some embodiments, the instructions to obtain the one or more endophenotype profiles further comprise instructions to access an aggregate of a plurality of gene interaction data to be utilized to construct one or more gene regulatory network graphs. In some embodiments, the plurality of gene interaction data comprises one or more known gene co-expression relationships, one or more known protein-to-protein interactions, one or more gene ontology relationships, or a combination thereof. In some embodiments, the one or more predicted interacting partner genes in the genome comprises one or more predicted interacting partner genes in a modified genotype of one or more plant seeds. In some embodiments, the machine-learning model was trained utilizing data representing information related to RNAseq, microarrays, ribosome profiling, single cell RNASeq, and/or proteome abundance. In some embodiments, the endophenotype comprises a tissue-specific gene endophenotype, a temporally-controlled gene endophenotype, or a change in gene endophenotype in response to a stimulus. In some embodiments, the genome editing platform is further configured to introduce the one or more modified genotypes to a plant based on the one or more predicted endophenotype profiles.

### BRIEF DESCRIPTION OF THE DRAWINGS

FIG. 1A illustrates an example embodiment of an exemplary genome editing platform and crop seed editing environment. A genome editing platform 100A is depicted on the left, in which the genome editing platform accesses a data set of gene regulatory sequences 104, which may be inputted to one or more trained machine-learning models 106. The one or more trained machine-learning models output one or more predicted endophenotype values 108. From this output, one or more desired gene regulatory sequences 110 are selected. The genome editing platform produces a guide listing 112 which may include one or more guide RNAs. A genome editing example 102 is depicted on the right. A guide RNA 115 is selected from the guide listing 112. The guide RNA 115 targets an endonuclease 114 to a targeted sequence 113. An edit is made in the targeted sequence 113, resulting in the desired gene regulatory sequence 116. The desired gene regulatory sequence is introduced into one or more crop seeds 117, which are used to germinate one or more crop plants 118.

FIG. 1B illustrates another example embodiment of an exemplary genome editing platform and crop seed editing environment. A genome editing platform 100B is depicted on the left, in which the genome editing platform accesses the gene-level endophenotype profiles of one or more target genotypes 120, which may be inputted to one or more trained machine-learning models 122. A first subset of endophenotype values 123 corresponding to a first subset of genes of the targeted genotype may be adjusted to desired values and inputted into the one or more trained machine-learning models 122. The one or more trained machine-learning models 122 output one or more predicted endophenotype values for unmodified genes 124. A gene regulatory network example 126 is depicted on the top right. Circles indicates nodes of the gene regulatory network, with each node representing an individual target gene. Nodes are connected by edges, depicted as lines connecting the circles. The network 128 may be inputted to one or more trained machine-learning models, depicted by arrows crossing a dotted line, the output of which is endophenotype values for the co-expressed interacting genes 130. Genes that are not co-expressed are indicated by black encompassing boxes in the top right.

FIG. 2 illustrates a flow diagram for generating an in silico prediction of endophenotype values corresponding to gene endophenotype for targeted genes identified for editing in response to a mutation of one or more cis regulatory sequences.

FIG. 3A illustrates an exemplary workflow diagram of an inference phase of a trained model for predicting endophenotype values for targeted genes identified for editing in response to a mutation of one or more cis regulatory sequences (including an evolutionarily constrained regulatory sequence data set).

FIG. 3B illustrates an exemplary workflow diagram of an inference phase of a trained model for predicting endophenotype values for targeted genes identified for editing in response to a mutation of one or more cis regulatory sequences (including a synthetic regulatory sequence data set).

FIG. 4A illustrates an exemplary workflow diagram of an initial stage of a model training phase for predicting endophenotype values for targeted genes identified for editing in response to a mutation of one or more cis regulatory sequences.

FIG. 4B illustrates an exemplary workflow diagram of a next stage of a model training phase for predicting endophenotype values for targeted genes identified for editing in response to a mutation of one or more cis regulatory sequences.

FIG. 4C illustrates an exemplary workflow diagram of a final stage of a model training phase for predicting endophenotype values for targeted genes identified for editing in response to a mutation of one or more cis regulatory sequences.

FIG. 5 illustrates a flow diagram for generating in silico predictions of endophenotype values corresponding to one or more co-expressed genes of a targeted genotype identified for editing in response to a mutation of one or more trans regulatory factors.

FIG. 6 illustrates an exemplary workflow diagram of an inference phase of a trained model for predicting endophenotype values corresponding to one or more co-expressed genes of a targeted genotype identified for editing in response to a mutation of one or more trans regulatory factors.

FIG. 7A illustrates an exemplary workflow diagram for a pre-processing stage of a training phase of a model for predicting endophenotype values corresponding to the gene co-expression for one or more co-expressed genes of a targeted genotype identified for editing in response to a mutation of one or more trans regulatory factors.

FIG. 7B illustrates an exemplary workflow diagram for a training stage of a training phase of a model for predicting endophenotype values corresponding to the gene co-expression for one or more co-expressed genes of a targeted genotype identified for editing in response to a mutation of one or more trans regulatory factors.

FIG. 8 illustrates a flow diagram for generating an in silico prediction of endophenotype values corresponding to one or more targeted genotypes identified for editing based a perturbation of a combination of one or more cis regulatory sequences and one or more trans regulatory factors.

FIG. 9A illustrates an exemplary workflow diagram of a training and inference phase of a promoter sequence to cis endophenotype effect model.

FIG. 9B illustrates an exemplary workflow diagram of a training and inference phase of a gene network-based trans endophenotype propagation model.

FIG. 9C illustrates an exemplary workflow diagram of an inference phase for predicting a gene-level endophenotype profile from a gene network and associated promoter sequences.

FIG. 10 illustrates an example genome editing computing system included as part of an exemplary genome editing platform.

FIG. 11 illustrates a diagram of an example artificial intelligence (AI) architecture included as part of an exemplary genome editing platform.

### DETAILED DESCRIPTION

The present embodiments are directed toward one or more computing devices of a genome editing platform that may be utilized to generate 1) an in silico prediction of endophenotype values corresponding to one or more targeted genes identified for editing in response to an upstream mutation of cis regulatory sequences corresponding to the one or more genes; 2) an in silico prediction of endophenotype values corresponding to a first set of one or more genes in response to a modulation in trans of the endophenotype values corresponding to a second set of one or more genes targeted for editing that interact with the first set of one or more genes; and 3) an in silico prediction of endophenotype values corresponding to a first set of one or more genes in response to a mutation of a second set of trans regulatory sequences corresponding to a second set of one or more genes.

In a first set of embodiments, the genome editing platform may be utilized to generate a gene regulatory sequence with a desired endophenotype profile that may be further utilized to modify an endophenotype in one or more plant seeds. In certain embodiments, the genome editing platform may generate one or more gene regulatory sequences. For example, in one embodiment, the one or more gene regulatory sequences may include one or more natural promoter sequences. In another embodiment, the one or more gene regulatory sequences may include one or more modified or synthetic gene regulatory sequences. In certain embodiments, the genome editing platform may then input the one or more gene regulatory sequences into a trained machine-learning model to obtain one or more variant effect predictions corresponding to one or more gene endophenotypes. For example, in some embodiments, the trained machine-learning model may include one or more sequence encoder models that may be utilized to generate predicted endophenotype values for the cis regulatory sequence of each input gene. In some embodiments, the one or more sequence encoder models may include language models used to perform natural language processing (NLP).

In certain embodiments, training the machine-learning model may include, for example, pre-training a randomly-initialized sequence encoder machine-learning model in a self-supervised manner by predicting randomly masked segments of a diverse set of gene regulatory sequences and backpropagating the prediction error through the model. In certain embodiments, training the machine-learning model may additionally include fine-tuning a previously pre-trained sequence encoder model in a self-supervised manner by predicting randomly masked segments of a targeted set of gene regulatory sequences collected from a particular species or gene family. In certain embodiments, training the machine-learning model may further include training a variant effect predictor machine-learning model in a supervised manner by predicting one or more experimentally observed endophenotype values using numerical features extracted from a previously trained sequence encoder model as input. In certain embodiments, training the variant effect predictor model may involve classification of discrete endophenotype categories, regression to numerical endophenotype values, or both. In certain embodiments, training the machine-learning model may further include utilizing the variant effect predictor model to predict one or more endophenotype measurements observed from one or more cell-based assays, one or more plant-based assays, or both. In certain embodiments, training the machine-learning model may further include fine-tuning the previously trained sequence encoder model by computing the error between endophenotype predictions and measurements, and backpropagating the result through the variant effect predictor model as well as the sequence encoder model.

In other embodiments, the trained machine-learning model may include one or more generative algorithms that may be utilized to sample one or more synthetic gene regulatory sequences from a learned distribution corresponding to a desired range of endophenotype profiles. In certain embodiments, the one or more generative algorithms may include a trained generative adversarial network (GAN), a trained variational autoencoder (VAE), or a Markov chain Monte Carlo (MCMC) sampling procedure. In certain embodiments, the genome editing platform may collect a plurality of natural cis regulatory sequences that are experimentally observed to have a desired effect on one or more endophenotypes. In some embodiments, the plurality of natural cis regulatory sequences that are experimentally observed to have a desired effect on one or more endophenotypes are input as seed sequences into the one or more space-sampling algorithms, In some embodiments, the genome editing platform may subsequently train one or more GANs and/or one or more VAEs to learn a distribution of gene regulatory sequences covering the desired range of endophenotypes, from which samples can then be drawn. In certain embodiments, the one or more trained GANs and/or one or more VAEs may be prompted to generate one or more novel synthetic gene regulatory sequences which correspond to a desired endophenotype profile. In other embodiments, an MCMC sampling algorithm may be used in conjunction with the trained sequence encoder model and trained variant effect predictor model to generate one or more novel synthetic gene regulatory sequences whose predicted endophenotypes are sufficiently likely to fall into the desired range according to some acceptance criteria. In some embodiments, a plurality of novel synthetic gene regulatory sequences generated by the one or more GANs and/or one or more VAEs are input as seed sequences into the one or more space-sampling algorithms. As used herein, a “seed sequence” refers to a sequence used as a seed in an algorithm. A seed sequence may be obtained from a plant seed or from a plant, or may be a synthetic sequence. In some embodiments, a plurality of novel synthetic gene regulatory sequences generated by the one or more GANs and/or one or more VAEs are input as seed sequences into the one or more space-sampling algorithms iteratively, until one or more gene regulatory sequences is outputted whose predicted endophenotypes are sufficiently likely to fall into the desired range according to some acceptance criteria.

In certain embodiments, subsequent to inputting the number of gene regulatory sequences into one or more trained machine-learning models to obtain the number of effect predictions corresponding to a number of endophenotypes, the genome editing platform may then select one or more desired endophenotypes from among the number of endophenotypes. For example, in one embodiment, the genome editing platform may select one or more desired endophenotype values from among the number of predicted endophenotype values that may be desired for downstream targeted editing of one or more of the number of gene regulatory sequences. In certain embodiments, the genome editing platform may then select the generated gene regulatory sequence corresponding to the one or more selected endophenotypes. For example, in certain embodiments, generating the gene regulatory sequence may include generating a gene regulatory sequence in accordance with a desired endophenotype value. In one embodiment, the desired endophenotype value may include a desired messenger RNA (mRNA) expression level. In some embodiments, the desired endophenotype is a tissue-specific gene endophenotype. A tissue-specific gene endophenotype refers to an endophenotype is a specified tissue. Desired tissue-specific gene endophenotype may include, but are not limited to, increasing transcription of a transcript in a tissue, decreasing transcription of a transcript in a tissue, increasing translation of a transcript in a tissue, decreasing translation of a transcript in a tissue, etc. For example, a desired tissue-specific endophenotype may be achieved by the introduction of a tissue-specific transcription factor binding site into the promoter of a gene of interest. In some embodiments, the desired endophenotype is a temporally-controlled gene endophenotype. A temporally-controlled gene endophenotype refers to an endophenotype that occurs at a specific time or stage in a plant lifespan, or in the cell cycle. For example, a desired temporally-controlled gene endophenotype may alter transcription, translation, or protein activity levels of a gene at various stages of the cell cycle, or may alter transcription, translation, or protein activity levels of a gene at a particular stage in a plant lifespan. For example, a selected gene regulatory sequence may induce a gene to be transcribed during the vegetative stage of growth, when it previously was not transcribed or was transcribed at low levels during the vegetative stage of growth. In some embodiments, the desired endophenotype is a change in gene endophenotype in response to a stimulus. In some embodiments, the change in gene endophenotype is in response to a biotic stimulus. In other embodiments, the change in gene endophenotype is in response to an abiotic stimulus. In some embodiments, the change in gene endophenotype is in response to a change in nutrient availability, a change in weather, herbivory, pests, infection, heat, cold, drought, flooding, salinity, or other stressors.

In certain embodiments, the genome editing platform may then generate a gene regulatory sequence in accordance with the one or more desired endophenotypes. For example, in certain embodiments, generating the gene regulatory sequence may include generating a gene regulatory sequence in accordance with a desired endophenotype level for use as a donor template nucleic acid. In certain embodiments, subsequent to generating the gene regulatory sequence in accordance with the one or more desired endophenotypes, the genome editing platform may then generate the sequence of one or more donor template nucleic acid molecules that comprise the gene regulatory sequence or a portion thereof. For example, in some embodiments, generating the one or more donor template nucleic acid molecules may include generating one or more donor template nucleic acid molecules configured to introduce a selected modified gene regulatory sequence to one or more plants, plant cells, or plant seeds. In some embodiments, the method comprises generating one or more guide RNAs (gRNAs). In some embodiments, the one or more gRNAs are designed to promote the introduction of the selected gene regulatory sequence into the targeted DNA sequence. In some embodiments, the one or more gRNAs are designed to induce a single-stranded or double-stranded break in the DNA near the targeted DNA sequence in order to promote DNA repair mechanisms, such as homology-directed repair, that will incorporate the selected gene regulatory sequence into the targeted DNA sequence, when a donor template nucleic acid comprising the selected gene regulatory sequence or a portion thereof is also provided. In certain embodiments, the generated one or more guides may be utilized to introduce the selected gene regulatory sequence into a plant and/or one or more plant seeds, thereby modifying the endophenotype of the plant and/or one or more plant seeds. In some embodiments, the genome editing platform may generate one or more guides that are gRNAs, and one or more donor template nucleic acids. In one embodiment, the genome editing platform generates a guide RNA that targets an endonuclease to the targeted gene sequence, and a donor template nucleic acid comprising the selected regulatory sequence to promote homologous recombination or another DNA repair mechanism to introduce the selected regulatory sequence into the genome of a plant and/or one or more plant seeds, thereby modifying the endophenotype of the plant and/or one or more plant seeds.

In a second set of embodiments, the genome editing platform may be utilized to generate a set of endophenotypes corresponding to one or more genes in a targeted genotype that may have a trans-regulatory effect on one or more non-overlapping genes in one or more plant seeds. In certain embodiments, the genome editing platform may obtain one or more endophenotypes corresponding to a gene. In certain embodiments, the genome editing platform may obtain one or more endophenotypes corresponding to each of a number of genes, resulting in one or more endophenotype profiles. For example, in certain embodiments, obtaining the one or more endophenotype profiles may include obtaining one or more endophenotype profiles corresponding to a target genotype.

In certain embodiments, the genome editing platform may then determine a first set of endophenotypes based on the one or more endophenotype profiles. In some embodiments, the first set of endophenotypes may correspond to a set of genes to be targeted for modification. In certain embodiments, the genome editing platform may then perform a desired modification to the first set of genes, in order to result in the first set of endophenotypes (e.g. defined by the user). In some embodiments, the genome editing platform may then input the modified first set of endophenotypes into a trained machine-learning model to obtain a prediction of a second set of endophenotypes, in which the second set of endophenotypes may correspond to a second, non-overlapping set of genes which interact with the first set of genes. For example, in certain embodiments, the genome editing platform may input the first set of endophenotypes into a trained machine-learning model by inputting one or more endophenotypes for each gene to the corresponding node of one or more graph neural networks (GNNs). In some embodiments, nodes of graphs corresponding to the one or more GNNs may represent genes associated with the genome, whereas edges of the graphs representing the one or more GNNs may represent predicted or experimentally-determined gene interactions. In some embodiments, the graphs corresponding to the one or more GNNs may be constructed by accessing an aggregate of a number of gene interaction data, in which the gene interaction data may include, for example, one or more known co-expressed genes, one or more known protein-protein interactions, one or more gene ontologies, or a combination thereof.

In certain embodiments, training the machine-learning model may include aggregating a dataset of endophenotype profiles of genotypes corresponding to a species, and selecting one or more random pairs of genotypes from the dataset with each pair corresponding to two distinct endophenotype profiles. In certain embodiments, training the machine-learning model may further include initializing one or more graph neural networks (GNNs) by randomly partitioning the nodes of graphs comprising the one or more GNNs into a first set of nodes corresponding to genes from a first genotype of the one or more random pairs of genotypes and a second set of nodes corresponding to non-overlapping genes from a second genotype of the one or more random pairs of genotypes. In certain embodiments, training the machine-learning model may further include inputting endophenotypes corresponding to the first genotype into the first set of nodes of the one or more GNNs and inputting endophenotypes corresponding to the second genotype into the second set of nodes of the one or more GNNs. In certain embodiments, the one or more GNNs may output predicted endophenotypes corresponding to the second genotype for the first set of nodes. In certain embodiments, the first genotype may correspond to the genotype of an unmodified plant seed. In certain embodiments, the second genotype may correspond to the genotype of a plant seed in which some genes have been modified. In certain embodiments, the first set of nodes of the one or more GNNs may correspond to genes which are unmodified in the first genotype, but have been modified in the second genotype. In certain embodiments, the second set of nodes of the one or more GNNs may correspond to genes which have not been modified in either the first genotype or the second genotype, but whose endophenotypes may or may not be affected via interactions with the genes that have been modified in the second genotype. In certain embodiments, the one or more predicted endophenotypes for the first genotype may include one or more predicted endophenotypes for a modified genotype of one or more plant seeds.

In certain embodiments, subsequent to inputting the first set of endophenotypes into a trained machine-learning model to obtain the prediction of a second set of endophenotypes, the genome editing platform may provide as feedback the second set of endophenotypes to the trained machine-learning model to refine the prediction of the second set of endophenotype levels in accordance with a predetermined evaluation metric. In certain embodiments, further subsequent to inputting the first set of endophenotypes into a trained machine-learning model to obtain the prediction of a second set of endophenotypes, the genome editing platform may modify the level of one or more endophenotypes of the second set of genes in one or more plant seeds indirectly in trans by directly modifying one or more endophenotypes in the first set of genes in cis.

In a third set of embodiments, the genome editing platform may be utilized to generate one or more gene-level endophenotype profiles based on an initial set of gene regulatory sequences that may be utilized to predict an effect of a mutated gene regulatory sequence of a first gene on the endophenotype of a second gene. In certain embodiments, the genome editing platform may input a number of gene regulatory sequences to a first trained machine-learning model, in which the number of gene regulatory sequences may include one or more mutated gene regulatory sequences. For example, in one embodiment, the first trained machine-learning model may include one or more sequence encoder models and one or more variant effect predictor models.

In certain embodiments, the genome editing platform may then utilize the first trained machine-learning model to generate a first set of gene-level endophenotype profiles based on the plurality of gene regulatory sequences. In certain embodiments, training the first machine-learning model may include pre-training a randomly-initialized sequence encoder model utilizing a self-supervised prediction of one or more gene regulatory sequences, and fine-tuning the pre-trained sequence encoder model utilizing a self-supervised prediction of a plurality of gene regulatory sequences extracted from a targeted species or gene family. In certain embodiments, training the first machine-learning model may further include utilizing a variant effect predictor model which performs a regression or classification using features extracted by the fine-tuned sequence encoder model to: 1) update the weights of the variant effect predictor model, 2) optionally update the weights of the sequence encoder model, and 3) generate effect predictions corresponding to a plurality of candidate endophenotypes of interest. In certain embodiments, training the first machine-learning model may further include utilizing the variant effect predictor model to predict a particular endophenotype measurement observed from one or more cell-based assays or one or more plant-based assays. In certain embodiments, training the variant effect predictor model may include computing a loss value based on a comparison of the effect predictions and the endophenotype measurement, and updating the weights based on a backpropagation of the computed loss value through the variant effect predictor model. In some embodiments, the sequence encoder model may be further fine-tuned by backpropagating the computed loss value through both the variant effect predictor model and the sequence encoder model.

In certain embodiments, subsequent to utilizing the first trained machine-learning model to generate a first set of gene-level endophenotype profiles, the genome editing platform may then input the first set of gene-level endophenotype profiles to a second trained machine-learning model. In certain embodiments, the genome editing platform may then utilize the second trained machine-learning model to generate a second set of gene-level endophenotype profiles based on the first set of gene-level endophenotype profiles. For example, in certain embodiments, generating the second set of gene-level endophenotype profiles may include predicting one or more gene-level endophenotype profiles based on one or more mutated gene regulatory sequences.

In certain embodiments, the second trained machine-learning model may include one or more graph neural networks (GNNs). In certain embodiments, the second machine-learning model may be trained by aggregating a dataset of endophenotype profiles of genotypes corresponding to a target species, and selecting one or more random pairs of genotypes from the dataset with each pair corresponding to two distinct endophenotype profiles. In certain embodiments, training the second machine-learning model may further include initializing the one or more graph neural networks (GNNs) by randomly partitioning nodes of graphs comprising the one or more GNNs into a first set of nodes corresponding to genes from a first genotype of the one or more random pairs of genotypes and a second set of nodes corresponding to non-overlapping genes from a second genotype of the one or more random pairs of genotypes. In certain embodiments, training the second machine-learning model may further include training the one or more GNNs based on endophenotypes corresponding to the first set of nodes and the second set of nodes.

In certain embodiments, the second set of gene-level endophenotype profiles may be predicted for a modified genotype of one or more plant seeds. In certain embodiments, subsequent to utilizing the second trained machine-learning model to generate the second set of gene-level endophenotype profiles based on the first set of gene-level endophenotype profiles, the genome editing platform may then provide as feedback the second set of gene-level endophenotype profiles to the second trained machine-learning model. For example, in some embodiments, providing as feedback the second set of gene-level endophenotype profiles to the second trained machine-learning model may include refining a prediction of the second set of gene-level endophenotype profiles in accordance with a predetermined evaluation metric. In certain embodiments, the second set of gene-level endophenotype profiles may be utilized to transform a plant and/or one or more plant seeds based on the second set of gene-level endophenotype profiles.

Accordingly, the present embodiments are directed toward one or more computing devices of a genome editing platform that may be utilized to generate 1) an in silico prediction of endophenotype values corresponding to one or more targeted genes identified for editing in response to an upstream mutation of one or more cis regulatory sequences; 2) an in silico prediction of endophenotype values corresponding to one or more genes of a targeted genotype in response to a perturbation of the endophenotypes of one or more interacting genes identified for editing, as a result of trans-regulatory effects; and 3) an in silico prediction of endophenotype values corresponding to one or more targeted genes in response to a mutation of the gene regulatory sequences of one or more interacting genes identified for editing, as a result of trans-regulatory effects. In this way, the present embodiments may facilitate and optimize genome editing in crop seeds (e.g., corn crop seeds, soybean crop seeds, rice crop seeds, wheat crop seeds, tomato crop seeds, citrus fruit crop seeds, cacao crop seeds, potato crop seeds, cotton crop seeds, cabbage crop seeds, mushroom crop seeds, canola crop seeds, papaya crop seeds, and so forth) and reduce unscalable phenotyping by being able to predict beforehand the outcome gene endophenotype profile for certain upstream mutations (e.g., substitutions, insertions, deletions, and so forth). By extension, the present embodiments may thus be employed to improve crop yields, increase tolerance to biotic and abiotic stresses, improve drought tolerance, increase tolerance to herbicides, improve pest repellency, improve seed oil composition for certain crop seeds, extension of shelf life of certain crop seeds, and so forth.

### Cis and Trans System-Level Overviews for Predicting Gene Endophenotypes

As used herein, the term “cis” is used to refer to the relation of two elements that are directly linked in some manner. A cis regulatory element refers to a DNA sequence that directly affects the transcription or translation of an associated gene. Cis regulatory elements include, but are not limited to, promoters, splicing donor sites, splicing acceptor sites, 5′ UTRs, 3′ UTRs, terminators, enhancers, activators, repressors, and transcription factor binding sites. A cis effect is the effect that a cis regulatory element has on the linked gene, transcript, or protein.

As used herein, the term “trans” is used to refer to the relation of two elements that are indirectly linked in some manner. A trans regulatory effect refers to the effect of a DNA sequence on the transcription or translation of a gene to which that DNA sequence is not directly or operably linked. For example, a cis regulatory element may increase the transcription of a first gene, wherein the first gene is a repressor of a second gene, such that increased transcription of the first gene leads to decreased transcription of a second gene; in this example, the regulatory element acts in cis in terms of its effect on the first gene, and has a trans regulatory effect on the second gene.

As used herein, the term “endophenotype” refers to a quantifiable phenotype at the sub-organismal level that can be measured by a biochemical, gene expression, or protein level assay, or by a visual feature measured at the sub-organismal level, e.g., via microscopy. In some embodiments, the endophenotype is an intermediate quantitative phenotype that is biologically relevant to, associated with, or predicative of a phenotype at the organism level, such as yield performance or overall fitness. Endophenotypes can be readily measured in cells, tissue, or young organisms that serve as a proxy to quickly determine which genetic variants are more likely to have an impact on a terminal phenotype, such as yield performance or overall fitness. Cell-based assays of endophenotype are assays performed on a cellular level, including but not limited to assays performed in or from cell culture, and assays performed on one or more individual cells (e.g. single-cell RNAseq, single-cell immunofluorescence, microscopy of cell culture, etc.). Plant-based assays of endophenotypes are assays performed on a tissue or organismal level (e.g. RNAseq of a tissue or plantlet, in situ hybridizations of a tissue, microscopy of a tissue, etc.). Examples of endophenotypes include, but are not limited to, messenger RNA (mRNA) abundance, gene transcript splicing ratio, protein abundance, micro RNA (miRNA) or small RNA (siRNA) abundance, translational efficiency, ribosome occupancy, protein modification, metabolite abundance, and allele specific expression (ASE), or combinations thereof. Endophenotypes may be associated with a genetic variant that is physically proximal or proximal within a gene network.

### Cis System-Level Overview

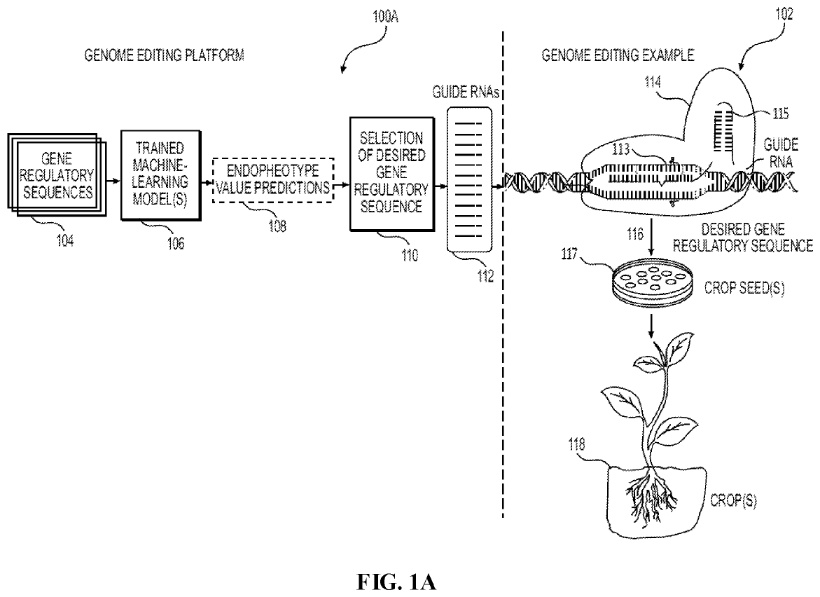

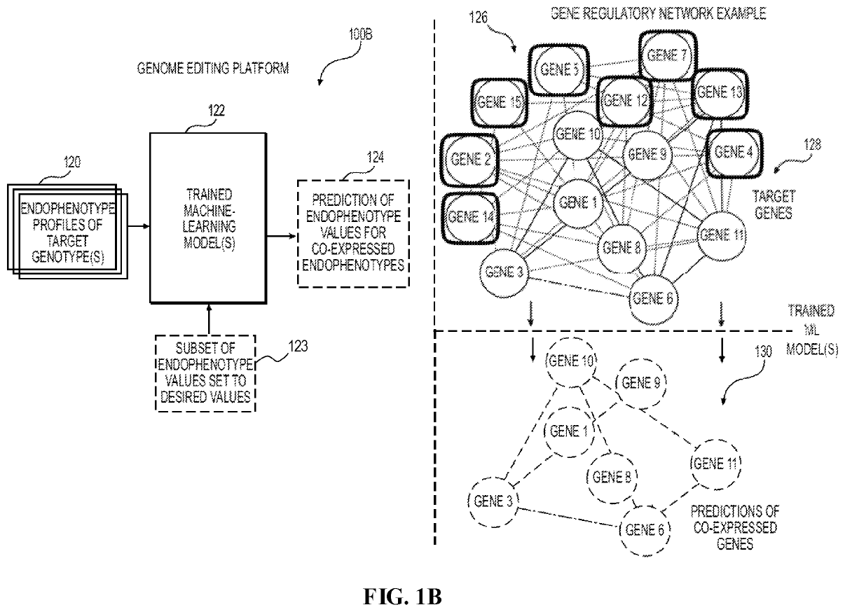

FIG. 1A illustrates an example embodiment of a genome editing platform and crop seed editing environment for generating an in silico prediction of endophenotype values corresponding to one or more targeted genes identified for editing in response to a mutation of one or more cis regulatory sequences, in accordance with the presently disclosed embodiments. As depicted, in certain embodiments, the genome editing platform and crop seed editing environment of FIG. 1A may include, for example, a genome editing platform 100A and a genome editing example 102. In certain embodiments, after models are trained, the genome editing platform 100A may access a data set of gene regulatory sequences 104. For example, in some embodiments, the data set of gene regulatory sequences 104 may include a genome assembly and annotation that may be pre-processed to extract an upstream promoter sequence, a terminator sequence, an untranslated region sequence (e.g., 3′UTR, 5′UTR), an intron sequence, or other cis regulatory sequence from each gene of the genome assembly.

In certain embodiments, the data set of gene regulatory sequences 104 may be inputted to one or more trained machine-learning models 106. For example, in some embodiments, the one or more trained machine-learning models 106 may include, for example, one or more sequence encoder model and one or more variant effect predictor model that may be utilized to output one or more predicted endophenotype values 108 for each gene of the genome assembly based on the inputted data set of gene regulatory sequences 104. In certain embodiments, the outputted one or more predicted endophenotype values 108 may include, for example, one or more qualitative biomarkers that may be indicative of an endophenotype level of one or more genes of the data set of gene regulatory sequences 104. In certain embodiments, one or more new gene regulatory sequences may be obtained by introducing mutations to the data set of gene regulatory sequences 104, and then iteratively feeding back the mutated data set of gene regulatory sequences into the one or more machine-learning models 106 models until a desired range of endophenotype values for each gene is predicted. In other embodiments, the one or more new gene regulatory sequences may be obtained automatically by sampling from a trained GAN or VAE, or by sampling using an MCMC algorithm, and then iteratively feeding back the mutated data set of gene regulatory sequences into the one or more machine-learning models 106 until a desired range of endophenotype values for each gene is predicted.

In certain embodiments, based on the output of one or more predicted endophenotype values 108, the genome editing platform 100A may then select a desired endophenotype level 110 that may be suitable for editing, for example, in one or more target genotype or individual genes of crop seeds (e.g., crop seeds 117) and/or plants (e.g., crop 118) downstream of the genome editing platform 100A in accordance with a desired endophenotype profile. As used herein, an “endophenotype profile” refers to a plurality of endophenotypes associated with an organism having a particular genotype. In some embodiments, the endophenotype profile is the entirety of endophenotypes. In some embodiments, the endophenotype profile is a plurality of endophenotypes related to a gene network or pathway. In some embodiments, the endophenotype profile is one or more endophenotypes. In some embodiments, the endophenotype profile is associated with an organism comprising a given complete genome sequence. In some embodiments, the endophenotype profile is associated with an organism comprising a specified genotype at a specified locus. In some embodiments, the endophenotype profile is associated with an organism comprising specified genotypes at more than one specified loci. For example, in certain embodiments, based on the selected desired endophenotype level 110, the genome editing platform 100A may then generate a guide listing 112, which may include, for example, one or more guide RNA (gRNAs) that may be suitable for identifying a target DNA region of interest and directing a nuclease or other enzyme thereto for editing genes at that specific region of interest. As used herein, a “gene” refers to a region of a genome that encodes a transcript, as well as regulatory regions that affect the transcription of the transcript and/or the abundance of a protein encoded by the transcript. Editing a gene, as used herein, can therefore reference editing the introns, exons, promoter, 5′ UTR, 3′ UTR, enhancers, repressors, and/or other regulatory regions that affect transcription levels of the transcript, translation levels of the transcript, degradation rates of the transcript, activity levels of the protein encoded by the transcript, etc. In one embodiment, the nuclease may be utilized, for example, to “cut” a target DNA sequence while being directed by the one or more gRNAs. As used herein, “downstream” may refer to a gene expression, gene editing, or other process that may be performed with respect to a gene regulatory sequence, for example, after a mutation or other process is performed with respect to a gene regulatory sequence. In some embodiments, the target DNA sequence may be subjected to further DNA editing, including but not limited to DNA nucleotide insertions, deletions, or substitutions.

As used herein “guide RNA” or “gRNA” refer to a nucleic acid that comprises or includes a nucleotide sequence (sometimes referred to a “spacer sequence”) that corresponds to (e.g., is identical or nearly identical to, or alternatively is complementary or nearly complementary to) a target DNA sequence (e.g., a contiguous nucleotide sequence that is to be modified) in a genome; the guide RNA functions in part to direct the CRISPR nuclease to a specific location on the genome. In embodiments, a gRNA is a CRISPR RNA (“crRNA”), such as the engineered Cas12a crRNAs described in this disclosure. For nucleases (such as a Cas9 nuclease) that require a combination of a trans-activating crRNA (“tracrRNA”) and a crRNA for the nuclease to cleave the target nucleotide sequence, the gRNA can be a tracrRNA: crRNA hybrid or duplex, or can be provided as a single guide RNA (sgRNA). At least 16 or 17 nucleotides of gRNA sequence corresponding to a target DNA sequence are required by Cas9 for DNA cleavage to occur; for Cas12a (Cpf1) at least 16 nucleotides of gRNA sequence corresponding to a target DNA sequence are needed to achieve detectable DNA cleavage and at least 18 nucleotides of gRNA sequence corresponding to a target DNA sequence were reported necessary for efficient DNA cleavage in vitro; see Zetsche et al. Cell 2015, 163:759-771. Cas12a (Cpf1) endonuclease and corresponding guide RNAs and PAM sites are disclosed in U.S. Pat. No. 9,790,490, which is incorporated herein by reference in its entirety and particularly for its disclosure of DNA encoding Cas12a (Cpf1) endonucleases and guide RNAs and PAM sites. In practice, guide RNA sequences are generally designed to contain a spacer sequence of between 17-24 contiguous nucleotides (frequently 19, 20, or 21 nucleotides) with exact complementarity (e.g., perfect base-pairing) to the targeted gene or nucleic acid sequence; guide RNAs having spacers with less than 100% complementarity to the target sequence can be used (e.g., a gRNA with a spacer having a length of 20 nucleotides and between 1-4 mismatches to the target sequence), but this can increase the potential for off-target effects. The design of effective guide RNAs for use in plant genome editing is disclosed in U.S. Patent Application Publication 2015/0082478 A1, the entire specification of which is incorporated herein by reference. Chemically modified sgRNAs have been demonstrated to be effective in Cas9 genome editing; see, for example, Hendel et al. Nature Biotechnol., 2015, 33:985-991.

In certain embodiments, during, for example, downstream implementation or experimentation, the generated guide listing 112 may be utilized to edit a targeted gene sequence 113, for example, as illustrated by the genome editing example 102. It should be appreciated that the genome editing example 102 may represent only a simplified example of a genome editing process and is included merely for the purposes of illustration. In accordance with the presently disclosed embodiments, any of various genome editing processes may be utilized during downstream implementation and/or experimentation of the present techniques. In one embodiment, the protein 114 may include, for example, a CRISPR associated protein (CAS) protein (e.g., Cas1 protein, Cas2 protein, Cas9 protein, Cas12 protein, CasX protein, CasY protein, and so forth). As depicted, a gRNA sequence 115 may identify a region of the targeted gene sequence 113 that may be edited (e.g., by a gene “knockout” technique, a gene “knock-in” technique, or other gene editing technique) in accordance with the predicted and desired gene endophenotype profile. In another embodiment, the protein 114 may include a zinc finger nuclease (ZFN) or a transcription activator-like effector nuclease (TALEN).

In some embodiments, the genome editing process further comprises a donor template nucleic acid. Donor template DNA molecules used in the aspects of the present disclosure provided herein include DNA molecules comprising, from 5′ to 3′, a first homology arm, a replacement DNA, and a second homology arm, wherein the homology arms containing sequences that are partially or completely homologous to genomic DNA (gDNA) sequences flanking a targeted gene sequence in the gDNA and wherein the replacement DNA can comprise an insertion, deletion, or substitution of 1 or more DNA base pairs relative to the target gDNA. In certain embodiments, a donor DNA template homology arm can be about 20, 50, 100, 200, 400, or 600 to about 800, or 1000 base pairs in length. In certain embodiments, a donor template DNA molecule can be delivered to a eukaryotic cell (e.g., a plant cell) in a circular (e.g., a plasmid or a viral vector including a geminivirus vector) or a linear DNA molecule. Donor DNA templates can be synthesized either chemically or enzymatically (e.g., in a polymerase chain reaction (PCR)).

In some embodiments, introduction of a selected gene regulatory sequence is accomplished through the use of PRIME editing (Anzalone et al. Nature 2019, 576 (7785): 149-157). In some embodiments, prime editing uses (i) a Cas nickase, in some embodiments a Cas9 nickase, in other embodiments a Cas12 nickase, fused to a reverse transcriptase (nCas-RT), in some embodiments a M-MLV reverse transcriptase, and (ii) a prime editing Cas guide RNA (pegRNA) that both specifies the genome target site and has an extension that encodes the target edit within a template for the reverse transcriptase. In some embodiments, the target edit is an insertion of a selected gene regulatory sequence. In some embodiments, the target edit is a deletion of one or more endogenous nucleotides to result in a selected gene regulatory sequence. In some embodiments, the target edit is a substitution of one or more endogenous nucleotides to result in a selected gene regulatory sequence. The binding of the pegRNA directs the Cas nickase to create a single-stranded break in the DNA at the nicking site. The extension of the pegRNA binds to the nicked DNA that has an exposed 3′-hydroxyl group, priming the reverse transcriptase to produce a DNA strand that is complementary to the extension of the pegRNA. This DNA strand will include the complement to any desired edits present in the provided pegRNA extension. Mismatch repair by the cell will then resolve the mismatch between the unedited parent strand and the edited product of the reverse transcriptase, thus introducing the desired edits into the genome. Prime editing systems may also include elements to inhibit mismatch repair, or to nick the unedited parent strand to increase editing efficiency. A mobility element can be fused to the pegRNA so as not to interfere with priming of the reverse transcriptase.

In some embodiments, prime editing can also be accomplished with Cas nucleases in place of Cas nickases (Adikusuma et al. Nucleic Acids Res. 2021, 49 (18): 10785-10795). In some embodiments, prime editing uses (i) a Cas nuclease, in some embodiments a Cas9 nuclease, in other embodiments a Cas12 nuclease, fused to a reverse transcriptase (Cas-RT), in some embodiments a M-MLV reverse transcriptase, and (ii) a prime editing Cas guide RNA (pegRNA) that both specifies the genome target site and has an extension that encodes the target edit within a template for the reverse transcriptase. In some embodiments, the binding of the pegRNA directs the Cas nuclease to create a double-stranded break in the DNA at the target site. The extension of the pegRNA binds to the cut DNA that has an exposed 3′-hydroxyl group, priming the reverse transcriptase to produce a DNA strand that is complementary to the extension of the pegRNA. This DNA strand will include the complement to any desired edits present in the provided pegRNA extension. Mismatch repair by the cell will then resolve the mismatch between the unedited parent strand and the edited product of the reverse transcriptase, thus introducing the desired edits into the genome. Prime editing systems may also include elements to inhibit mismatch repair, or to nick the unedited parent strand to increase editing efficiency. A mobility element can be fused to the pegRNA so as not to interfere with priming of the reverse transcriptase.

Prime editing makes precise DNA sequence modifications rather than random insertions, deletions, and substitutions (Indels), thus increasing the probability of obtaining the desired effect. Prime editing may be used to introduce any single base pair substitution as well as small deletion or insertions. Deletions of up to 80 base pairs have been produced using prime editing with a single pegRNA in human cells, and insertions of up to 40 base pairs (Anzalone et al. Nature 2019, 576:149-157). Dual pegRNA systems are also known in the art (Choi et al. Nat Biotechnol 2021, 40 (2): 218-226; Lin et al. Nature Biotechnology 2021, 39 (8): 923-927) and can be used to generate precise large deletions, or to improve editing efficiency for small insertions, deletions, or substitutions. Additionally, dual pegRNA systems where the extensions of the pegRNAs are not complementary to the endogenous locus, but are complementary to one another, can be used to replace endogenous sequence and/or mediate larger insertions (Anzalone et al. Nat Biotechnol 2022, 40 (5): 731-740).

In some embodiments, the Cas nuclease is associated with a reverse transcriptase. In some embodiments, the Cas nuclease is fused to the reverse transcriptase. In some embodiments, the guide RNA comprises at its 3′ end a priming site and an edit to be incorporated into the genomic target. In some embodiments, the Cas nuclease is a Cas nickase. In some embodiments, the Cas nickase is a Cas9 nickase or a Cas12 nickase. In some embodiments, the Cas nickase comprises mutation in one or more nuclease active sites.

In certain embodiments, a desired gene regulatory sequence 116 may be generated and introduced to one or more crop seeds 117 (e.g., corn crop seeds, soybean crop seeds, rice crop seeds, wheat crop seeds, tomato crop seeds, citrus fruit crop seeds, cacao crop seeds, potato crop seeds, cotton crop seeds, cabbage crop seeds, mushroom crop seeds, canola crop seeds, papaya crop seeds, and so forth) to germinate one or more crops 118 (e.g., corn crop, soybean crop, rice crop, wheat crop, tomato crop, citrus fruit crop, cacao crop, potato crop, cotton crop, cabbage crop, mushroom crop, canola crop, papaya crop, and so forth) in accordance with the desired gene regulatory sequence 116. As used herein, “introducing,” “introduction,” or to “introduce” refer to any method requiring human intervention which results in a selected nucleic acid sequence being present in a plant's genome that was not originally present in the plant's genome at that locus. This includes, but is not limited to, adding the nucleic acid sequence to a plant genome de novo, deleting endogenous DNA to result in the nucleic acid sequence, and modifying and/or editing an existing DNA sequence to result in the nucleic acid sequence.

Vectors are used to deliver nucleic acids to plant cells. In some embodiments, the vector is capable of autonomous replication within the host cell. In other embodiments, the vector is integrated into the genome of the host cell and replicated with the host genome. In some embodiments, termed “expression vectors”, the genes of the vector are expressed or are capable of being expressed under certain conditions. In some embodiments, the vector contains a gene regulatory sequence selected through the method of an aspect of the present disclosure. In some embodiments, the vector contains a gene regulatory sequence selected through the method of an aspect of the present disclosure, operably linked to a gene. In some embodiments, the vector contains one or more regulatory elements operably linked to a gene. In some embodiments, the vector contains a promoter. In some embodiments, the promoter is a constitutive promoter, a conditional promoter, an inducible promoter, or a temporally or spatially specific promoter (e.g., a tissue specific promoter, a developmentally regulated promoter, or a cell cycle regulated promoter). In some embodiments, a vector is introduced to a host cell to produce RNA transcripts, proteins, or peptides within the host cell, as encoded by the contained nucleic acid.

In embodiments of the method, the selected gene regulatory sequence and/or the components of the genomic editing platform are delivered via at least one viral vector selected from the group consisting of adenoviruses, lentiviruses, adeno-associated viruses, retroviruses, geminiviruses, begomoviruses, tobamoviruses, potex viruses, comoviruses, wheat streak mosaic virus, barley stripe mosaic virus, bean yellow dwarf virus, bean pod mottle virus, cabbage leaf curl virus, beet curly top virus, tobacco yellow dwarf virus, tobacco rattle virus, potato virus X, and cowpea mosaic virus. In embodiments of the method, the selected gene regulatory sequence and/or the components of the genomic editing platform are delivered via at least one bacterial vector capable of transforming a plant cell and selected from the group consisting of Agrobacterium sp., Rhizobium sp., Sinorhizobium (Ensifer) sp., Mesorhizobium sp., Bradyrhizobium sp., Azobacter sp., and Phyllobacterium sp. In some embodiments, a viral vector may be delivered to a plant by transformation with Agrobacterium.

In another embodiment, a T-DNA vector is used to deliver at least one nucleic acid to plant cells. In some embodiments, a T-DNA binary vector is used. In some embodiments, a T-DNA superbinary vector system is used. In other embodiments, a T-DNA ternary vector system is used. In some embodiments, the T-DNA system further comprises an additional virulence gene cluster. In some embodiments, the T-DNA system further comprises an accessory plasmid or virulence helper plasmid. In some embodiments, the T-DNA vector is an Agrobacterium vector.

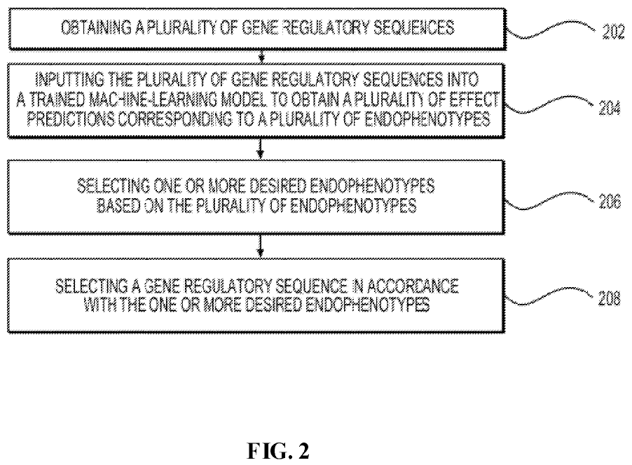

In this way, and as will be further appreciated with respect to FIGS. 2-9C below, the present embodiments may facilitate and optimize genome editing in crop seeds (e.g., corn crop seeds, soybean crop seeds, rice crop seeds, wheat crop seeds, tomato crop seeds, citrus fruit crop seeds, cacao crop seeds, potato crop seeds, cotton crop seeds, cabbage crop seeds, mushroom crop seeds, canola crop seeds, papaya crop seeds, and so forth) and reduce unscalable phenotyping by being able to predict beforehand the outcome gene endophenotype profile for a certain upstream mutation (e.g., substitutions, insertions, deletions, and so forth). By extension, the present embodiments may thus be employed to improve crop yields, increase tolerance to biotic and abiotic stresses, improve drought tolerance, increase tolerance to herbicides, improve pest repellency, improve seed oil composition for certain crop seeds, extension of shelf life of certain crop seeds, and so forth, in comparison to a control plant that has not been subjected to or modified by the present embodiments. As used herein, “upstream” may refer to a mutation or other process that may be performed with respect to a gene regulatory sequence, for example, prior to any gene expression or gene editing processes performed with respect to a gene regulatory sequence.

### Trans System-Level Overview

FIG. 1B illustrates another example embodiment of a genome editing platform and crop seed editing environment, in accordance with the presently disclosed embodiments. As depicted, in certain embodiments, the genome editing platform and crop seed editing environment of FIG. 1B may include, for example, a genome editing platform 100B and a gene regulatory network example 126. In certain embodiments, after models are trained, the genome editing platform 100B may have access to the gene-level endophenotype profile of a targeted genotype 120. For example, in some embodiments, the gene-level endophenotype profile of a targeted genotype 120 may include, for example, one or more endophenotypes for all or a subset of genes corresponding to the target genotype to be modified, which contribute to its overall phenotype.

In certain embodiments, the endophenotype profile of a targeted genotype 120 may be inputted to one or more trained machine-learning models 122, in which a first subset of endophenotype values 123 corresponding to a first subset of genes of the targeted genotype may be adjusted (e.g., by user input) to desired values, and in which a second subset of endophenotype values corresponding to a second subset of genes. For example, in some embodiments, the first subset of endophenotype values 123 adjusted to the desired values may correspond, for example, to a set of genes that can be targeted and edited by the gene editing platform (e.g., by a gene “knockout” technique, a gene “knock-in” technique, base editing, or other gene editing technique). In addition, in some embodiments, the one or more trained machine-learning models 122 may include, for example, one or more GNN models that may be utilized to output predictions for one or more updated endophenotype values 124 (e.g., indicative of the endophenotype level) for a second subset of interacting genes based on the inputted adjusted first subset of endophenotype values and the inputted initial second subset of endophenotype values of data set of endophenotype profiles of a targeted genotype 120. Specifically, the outputted one or more predicted endophenotype values 124 may include the updated endophenotype values for the second subset of genes which interact with the first subset of genes whose endophenotype values 123 were previously set to desired values.

In certain embodiments, the gene regulatory network example 126 may represent an illustrative example of the forgoing embodiments. For example, as depicted, a gene regulatory network 126 may include, for example, a graph including nodes representing target genes (e.g., “Gene 1”, “Gene 2”, “Gene 3”, “Gene 4”, “Gene 5”, “Gene 6”, “Gene 7”, “Gene 8”, “Gene 9”, “Gene 10”, “Gene 11”, “Gene 12”, “Gene 13”, and “Gene 14”). In some embodiments, one or more of the nodes representing target genes (e.g., “Gene 1”, “Gene 2”, “Gene 3”, “Gene 4”, “Gene 5”, “Gene 6”, “Gene 7”, “Gene 8”, “Gene 9”, “Gene 10”, “Gene 11”, “Gene 12”, “Gene 13”, and “Gene 14”) may be set to desired values. As further depicted, the gene regulatory network 128 may be then inputted to the one or more trained machine-learning models 122, and the one or more trained machine-learning models 122 may output endophenotype values for the co-expressed interacting genes 130 (e.g., “Gene 1”, “Gene 3”, “Gene 6”, “Gene 8”, “Gene 9”, “Gene 10”, and “Gene 11”) to the respective genes of the subset of endophenotype values 123 previously set to desired values. In certain embodiments, the outputted endophenotype values for the co-expressed interacting genes 130 (e.g., “Gene 1”, “Gene 3”, “Gene 6”, “Gene 8”, “Gene 9”, “Gene 10”, and “Gene 11”) may be utilized, for example, to facilitate and optimize genome editing that may be performed downstream with respect to, for example, the one or more crop seeds 117 and/or the one or more crops 118 as previously discussed above with respect to FIG. 1A.

Thus, in accordance with the presently disclosed embodiments, the present techniques as illustrated by FIG. 1A and FIG. 1B, respectively, are directed toward one or more computing devices of a genome editing platform that may be utilized to generate 1) an in silico prediction of endophenotype values corresponding to one or more targeted genes identified for editing in response to an upstream mutation of one or more cis regulatory sequences; 2) an in silico prediction of endophenotype values corresponding to one or more genes of a targeted genotype in response to a mutation of the endophenotypes of one or more interacting genes identified for editing, as a result of trans-regulatory effects; and 3) an in silico prediction of endophenotype values corresponding to one or more targeted genes in response to a mutation of the gene regulatory sequences of one or more interacting genes identified for editing, as a result of trans-regulatory effects. In this way, the present embodiments may facilitate and optimize genome editing in crop seeds (e.g., corn crop seeds, soybean crop seeds, rice crop seeds, wheat crop seeds, tomato crop seeds, citrus fruit crop seeds, cacao crop seeds, potato crop seeds, cotton crop seeds, cabbage crop seeds, mushroom crop seeds, canola crop seeds, papaya crop seeds, and so forth) and reduce unscalable phenotyping by being able to predict beforehand the outcome gene endophenotype profile for certain upstream perturbations (e.g., modifications, mutations, and so forth). By extension, the present embodiments may thus be employed to improve crop yields, increase tolerance to biotic and abiotic stresses, improve drought tolerance, increase tolerance to herbicides, improve pest repellency, improve seed oil composition for certain crop seeds, extension of shelf life of certain crop seeds, and so forth.

### Predicting Gene Endophenotypes Based on Mutations in Cis Regulatory Sequences

### Cis Endophenotype Model Inference Phase

FIG. 2 illustrates a flow diagram 200 for generating an in silico prediction of endophenotype values corresponding to one or more targeted genes identified for editing in response to a mutation of one or more cis regulatory sequences, in accordance with the presently disclosed embodiments. The flow diagram 200 may be performed utilizing one or more processing devices (e.g., genome editing platform 100A) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), tensor processing unit (TPU), or any other processing device(s) that may be suitable for processing genomics data or other omics data), software (e.g., instructions running/executing on one or more processors), firmware (e.g., microcode), or some combination thereof.

The flow diagram 200 may begin at block 202 with one or more processing devices (e.g., genome editing platform 100A) obtaining a plurality of gene regulatory sequences. The flow diagram 200 may then continue at block 204 with one or more processing devices (e.g., genome editing platform 100A) inputting the plurality of gene regulatory sequences into a machine-learning model trained to obtain a plurality of effect predictions corresponding to a plurality of endophenotypes. The flow diagram 200 may then continue at block 206 with one or more processing devices (e.g., genome editing platform 100A) selecting one or more desired endophenotypes based on the plurality of endophenotypes. The flow diagram 200 may then conclude at block 208 with one or more processing devices (e.g., genome editing platform 100A) selecting a gene regulatory sequence in accordance with the one or more desired endophenotypes.

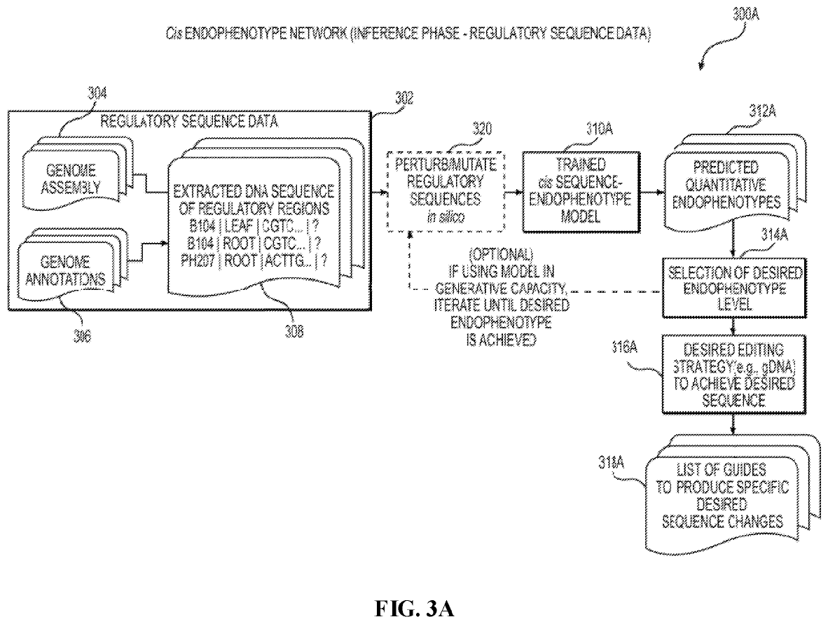

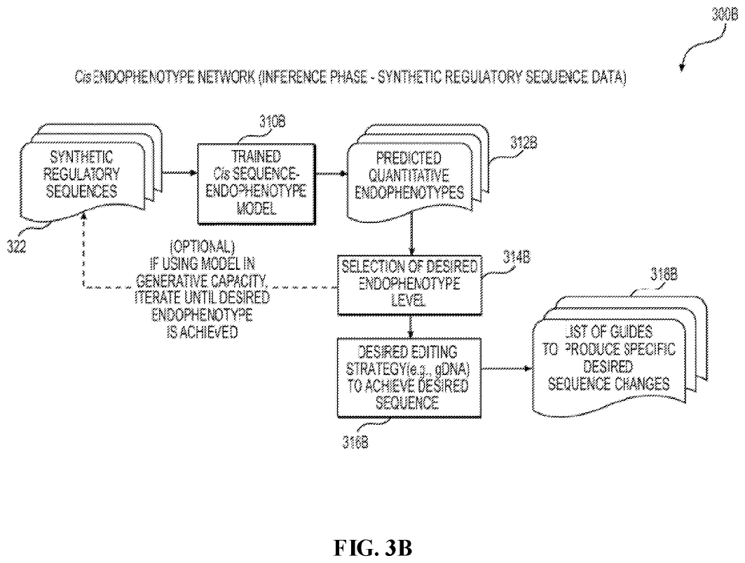

FIG. 3A gene endophenotype illustrates an exemplary workflow diagram 300A of an inference phase of a trained model for predicting endophenotype values for targeted genes identified for editing in response to a mutation of one or more cis regulatory sequences (including an evolutionarily constrained regulatory sequence data set), in accordance with the presently disclosed embodiments. In certain embodiments, the workflow diagram 300A may begin with obtaining a regulatory data set 302. In certain embodiments, the regulatory data set 302 may include a genome assembly 304 and genome annotations 306. In certain embodiments, the genome assembly 304 and genome annotations 306 may include, for example, a large, curated data set of naturally-occurring gene-proximal putative raw regulatory sequences that may or may not be evolutionarily constrained and defined. In some embodiments, the genome assembly 304 and genome annotations 306 may be obtained, for example, by extracting cis regulatory sequences from one or more public or proprietary reference genome sequences and annotations that indicate the coordinates of each gene.

In certain embodiments, a data set of extracted DNA sequence of regulatory regions 306 may include, for example, one or more promoter sequences, terminator sequences, UTR sequences (e.g., 3′UTR, 5′UTR), intron sequences, or other cis regulatory sequences that may be extracted from the genome assembly 304 and labeled based on the genome annotations 306. In certain embodiments the data set of extracted DNA sequence of regulatory regions 306 may be then inputted to the one or more trained machine-learning models 310A. In some embodiments, the one or more trained machine-learning models 310A may include, for example, one or more sequence encoder models (e.g., one or more sequence-to-sequence (seq2seq) machine-learning models, one or more transformer-based machine-learning models, or one or more other encoder-based machine translation language models) that may be utilized to generate predictions of endophenotype values 312A (e.g., qualitative biomarker or other measurable value) based on the inputted data set of extracted DNA sequence of regulatory regions 306. In other embodiments, the trained machine-learning model may include one or more generative algorithms that may be utilized to sample one or more synthetic gene regulatory sequences from a learned distribution corresponding to a range of desired endophenotype values 312A. In certain embodiments, the one or more generative algorithms may include a trained generative adversarial network (GAN), a trained variational autoencoder (VAE), or a Markov chain Monte Carlo (MCMC) sampling procedure. In certain embodiments, the genome editing platform may collect a plurality of natural cis regulatory sequences that are experimentally observed to have a desired effect on one or more endophenotypes. In some embodiments, the one or more GANs and/or one or more VAEs may be trained to learn a distribution of gene regulatory sequences covering the range of desired endophenotype values 312A, from which samples can then be drawn. In certain embodiments, the one or more trained GANs and/or one or more VAEs may be prompted to generate one or more novel synthetic gene regulatory sequences which correspond to a desired endophenotype profile. In other embodiments, an MCMC sampling algorithm may be used in conjunction with the trained sequence encoder model and trained variant effect predictor model to generate one or more novel synthetic gene regulatory sequences whose predicted endophenotypes are sufficiently likely to fall into the desired range according to some acceptance criteria.

In certain embodiments, based on the predictions of endophenotype values 312A generated by the one or more trained machine-learning models 310A, one or more selections 314A (e.g., via one or more user inputs) of a desired endophenotype level may be received. In certain embodiments, based on the selection of the desired endophenotype level, a genome editing strategy 316A may be generated. For example, in some embodiments, the genome editing strategy 316A may include one or more generated gRNAs that may be used to facilitate an editing of one or more target genes. In certain embodiments, as further depicted, based on the genome editing strategy 316A, the list of gRNAs may be produced to perform one or more gene edits for producing a desired gene regulatory sequence.

FIG. 3B illustrates an exemplary workflow diagram 300B of an inference phase of a trained model for predicting endophenotype values for targeted genes identified for editing in response to a mutation of one or more cis regulatory sequences (including a synthetic regulatory sequence data set), in accordance with the presently disclosed embodiments. In certain embodiments, the workflow diagram 300B may begin with obtaining a data set of regulatory sequences 322. In certain embodiments, the data set of regulatory sequences 322 may include, for example, a set of synthetic promoter sequences that may be designed based on one or more end-user preferences. In certain embodiments the data set of regulatory sequences 322 may be then inputted to the one or more trained machine-learning models 310B.

In some embodiments, the one or more trained machine-learning models 310B may include, for example, one or more sequence encoder models and one or more variant effect predictor models that may be utilized to generate predictions of endophenotype values 312B (e.g., qualitative biomarker or other measurable value) based on the inputted data set of extracted DNA sequence of regulatory regions 306. In other embodiments, the trained machine-learning model may include one or more generative algorithms that may be utilized to sample one or more synthetic gene regulatory sequences from a learned distribution corresponding to a range of desired endophenotype values 312B. In certain embodiments, the one or more generative algorithms may include a trained GAN, a trained VAE, or an MCMC sampling procedure. In certain embodiments, the genome editing platform may collect a plurality of natural cis regulatory sequences that are experimentally observed to have a desired effect on one or more endophenotypes. In some embodiments, the genome editing platform may subsequently train one or more GANs and/or one or more VAEs to learn a distribution of gene regulatory sequences covering the desired range of endophenotype values 312B

In certain embodiments, based on the predictions of endophenotype values 312B generated by the one or more trained machine-learning models 310B, one or more selections 314B (e.g., via one or more user inputs) of a desired endophenotype level may be received. In certain embodiments, based on the selection of the desired endophenotype level, a genome editing strategy 316B may be generated. For example, in some embodiments, the genome editing strategy 316B may include one or more generated gRNAs that may be used to facilitate an editing of one or more target genes. In certain embodiments, as further depicted, based on the genome editing strategy 316B, the list of gRNAs may be produced to perform one or more gene edits for producing a desired gene regulatory sequence.

### Cis Endophenotype Model Training Phase

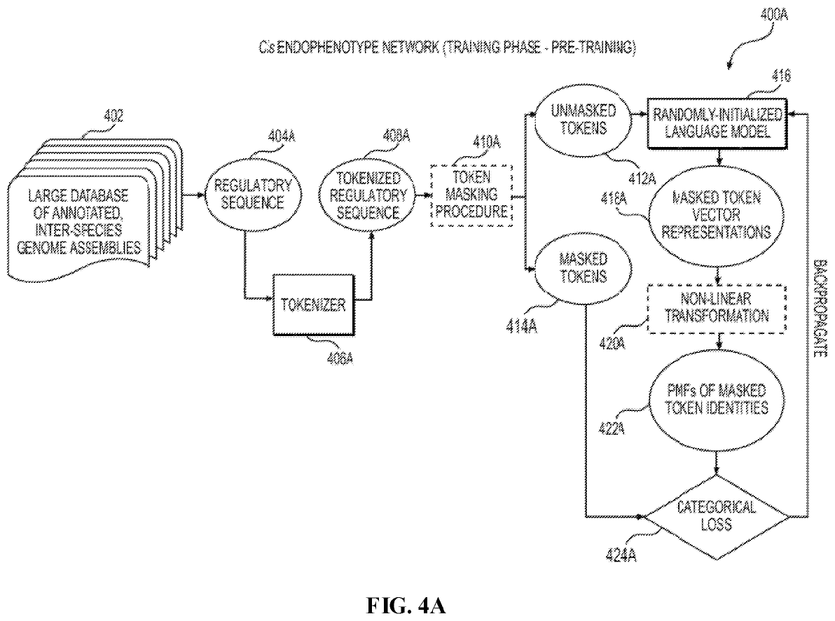

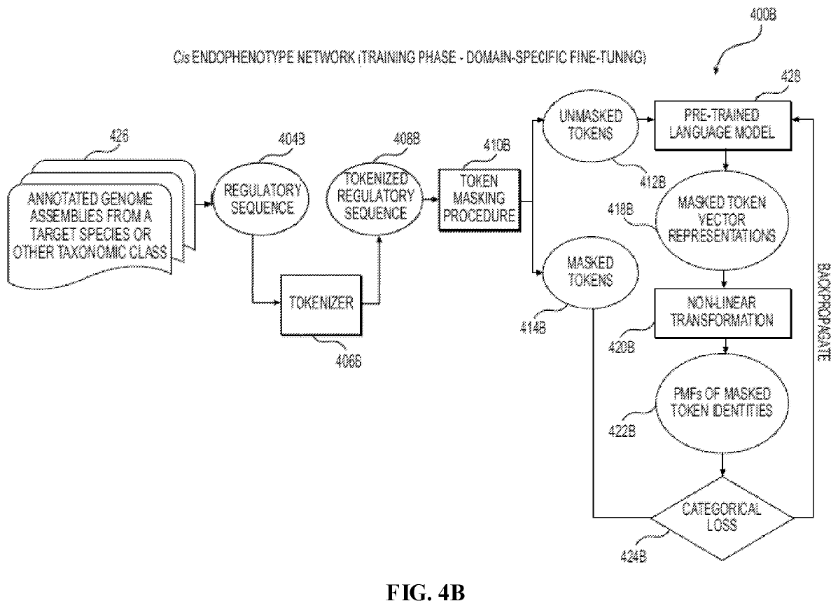

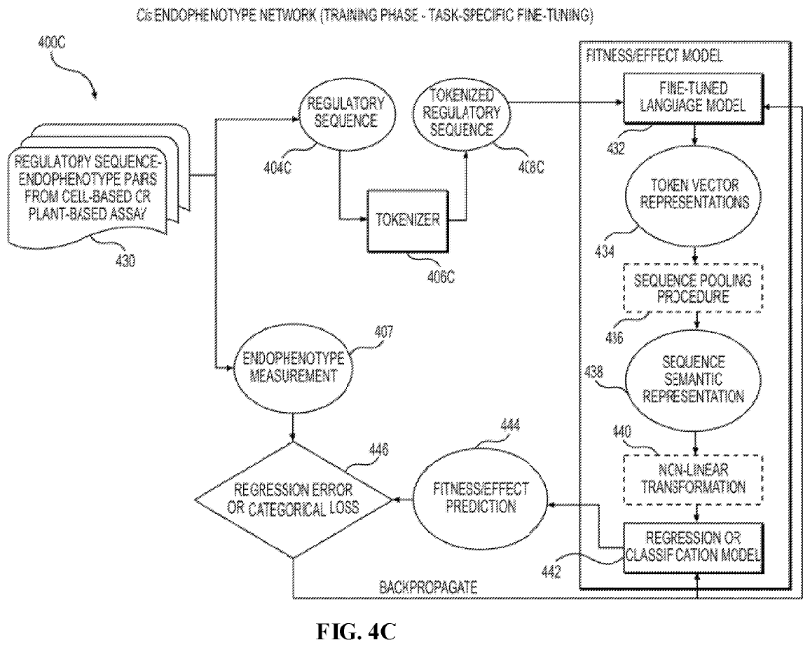

FIG. 4A illustrates an exemplary workflow diagram 400A of an initial stage of a model training phase for predicting endophenotype values for targeted genes identified for editing in response to a mutation of one or more cis regulatory sequences, in accordance with the presently disclosed embodiments. In certain embodiments, the workflow diagram 400A may begin with obtaining a training data set of annotated inter-species genome assemblies 402. For example, in some embodiments, the training data set of annotated inter-species genome assemblies 402 may include, for example, a large, curated dataset of naturally-occurring gene-proximal putative raw regulatory sequences that may or may not be evolutionarily constrained.

In certain embodiments, the workflow diagram 400A may then proceed with extracting a data set of regulatory sequences 404A from the training data set of annotated inter-species genome assemblies 402. For example, in some embodiments, the extracted regulatory sequences 404A may include, for example, one or more promoter sequences, terminator sequences, UTR sequences (e.g., 3′UTR, 5′UTR), intron sequences, or other cis regulatory sequences that may be extracted from the training data set of annotated inter-species genome assemblies 402. In certain embodiments, the promoter sequences 404A may be obtained by extracting cis regulatory sequences, for example, from public or proprietary reference genome sequences and annotations that indicate the coordinates of each gene included in the training data set of annotated inter-species genome assemblies 402. In one embodiment, the regulatory sequences 404A may be further filtered using minimum sequence similarity cutoff in order to prevent overfitting when provided to one or more trained machine-learning models.

In certain embodiments, the regulatory sequences 404A may be inputted to a tokenizer 406A. For example, in certain embodiments, the tokenizer 406A may include any functional process that may be suitable for deconstructing the regulatory sequences 404A or other sequences of textual data (e.g., gene bases “ATGACGGATCAGCCGGCAA” . . . (SEQ ID NO: 1)) into subsets of “tokens” (e.g., “ATGA”, “CGGA”, “TCAG”, and so forth (e.g., equivalent to deconstructing a sentence into individual phrases or individual words)). In certain embodiments, as further depicted by the workflow diagram 400A of FIG. 4A, the tokenizer 406A may then output a set of tokenized regulatory sequences 408A. In certain embodiments, the workflow diagram 400A may then proceed with performing a token masking process 410A based on the set of tokenized regulatory sequences 408A. For example, in certain embodiments, the token masking process 410A may include any process that may be suitable for performing, for example, a fill-in-the-blank operation (e.g., based on a prediction of missing nucleotides and/or sequences of nucleotides), in which the token masking process 410A may utilize the gene bases surrounding the tokens of the set of tokenized regulatory sequences 408A for predicting the gene base of which the masked token is to be labeled or assigned (e.g., bounded by evolutionary constraints). In one embodiment, the masked tokens 414A may be then utilized as ground truth data for training a randomly-initialized language machine-learning model 416 that utilizes a sequence of unmasked tokens 412A as input data.

In certain embodiments, the randomly-initialized language ML model 416 may include, for example, one or more sequence encoder models (e.g., including one or more masked language models (MLMs), one or more causal language models (CLMs), one or more next sentence prediction models, one or more next word prediction models, transformer-based machine-learning models, or other language model) that may be utilized to predict gene bases that may have been masked in the input sequence of unmasked tokens 412A. For example, in certain embodiments, the randomly-initialized language ML model 416 may include one or more sequence encoder models that train, for example, in a self-supervised manner on batches of the unmasked tokens 412A as input and the masked tokens 414A as ground truth. The randomly-initialized language ML model 416 may be then evaluated (e.g., rewarded or penalized) based on its ability to successfully predict gene bases that have been masked in the input sequence of unmasked tokens 412A and then updated the model parameters to minimize the calculated loss of the randomly-initialized language ML model 416 after each iteration.

For example, as further depicted by the workflow diagram 400A, the randomly-initialized language ML model 416 may generate a prediction of masked token vector representations 418A. In certain embodiments, the randomly-initialized language ML model 416 may generate the prediction of masked token vector representations 418A based on, for example, self-learned grammar, semantics, and syntax of the input sequence of unmasked tokens 412A bounded by evolutionary constraints. For example, due to the evolutionary constraints imposed upon the input sequence of unmasked tokens 412A bounded, the internal state or parameterization of the randomly-initialized language ML model 416 may be configured to approximate the distribution of sequential and evolutionarily-sampled runs of gene base pairs. Under the assumption of independent and identically distributed training data, the approximation may become increasingly accurate in the large data limit. Additionally, because the randomly-initialized language ML model 416 may only fit a conditional probability distribution based on the sequence space sampled in the dataset, the dependence of predictions on physical interactions with the environment is implicit.

In certain embodiments, the conditional probability distribution may include a parameterization defined by a learned set of semantic features, which together form the vector representations 418A. In one embodiment, such semantic features may later be interrogated for pertinence to the variation in endophenotype of a gene over various tissues, developmental stages, or in response to a specific stress stimulus or other perturbations. In certain embodiments, the workflow diagram 400A may then proceed with performing a non-linear transformation 420A of the vector representations 418A and generating one or more probability mass functions (PMFs) of the identities of the masked tokens 422A. For example, in some embodiments, the one or more PMFs of the identities of the masked tokens 422A may represent a function that maps a class label of a respective masked token 422A to the probability of the respective masked token 422A actually taking on that class label. In certain embodiments, the workflow diagram 400A may then proceed with calculating a categorical loss 424A based on the one or more PMFs of the identities of the masked tokens 422A. For example, in some embodiments, the categorical loss 424A may include one or more functions that may be utilized for evaluating how correct or how incorrect a predicted class label for respective unmasked tokens 412A is by comparing against the ground truth masked tokens 414A and then updating (e.g., via backpropagation) the randomly-initialized language ML model 416 based thereon. As an example, in simplest of terms, the foregoing process 400A may, at least in one embodiment, be equivalent to masking a few words of a sentence and then utilizing the randomly-initialized language ML model 416 to predict those masked words based on other unmasked words in that same sentence.

FIG. 4B illustrates an exemplary workflow diagram 400B of a next stage of a model training phase for predicting endophenotype values for targeted genes identified for editing in response to a mutation of one or more cis regulatory sequences gene endophenotype, in accordance with the presently disclosed embodiments. Specifically, the workflow diagram 400B may be suitable for utilizing a self-supervised sequence encoder model (e.g., pre-trained language ML model 428 corresponding, in one embodiment, to the randomly-initialized language ML model 416) trained to predict the sequence content of each individual regulatory sequence subcomponent (e.g., promoters, 3′UTR, 5′UTR, CDS, introns, post terminators, and so forth). For example, in certain embodiments, the workflow diagram 400B may begin with obtaining a training data set of annotated genome assemblies collected from a targeted gene species or other taxonomic class 426.

In certain embodiments, the workflow diagram 400B may then proceed with extracting a data set of regulatory sequences 404B extracted from the training data set of annotated genome assemblies collected from a targeted gene species 426. For example, in some embodiments, the extracted regulatory sequences 404B may include, for example, one or more promoter sequences, terminator sequences, UTR sequences (e.g., 3′UTR, 5′UTR), intron sequences, or other cis regulatory sequences that may be extracted from the training data set of annotated genome assemblies collected from a targeted gene species 426. In certain embodiments, the promoter sequences 404B may be obtained by extracting cis regulatory sequences, for example, from public or proprietary reference genome sequences and annotations that indicate the coordinates of each gene included in the training data set of annotated genome assemblies collected from a targeted gene species 426. In one embodiment, the regulatory sequences 404B may be further filtered using minimum sequence similarity cutoff in order to prevent overfitting when provided to one or more trained machine-learning models.

In certain embodiments, the regulatory sequences 404B may be then inputted to a tokenizer 406B. For example, in certain embodiments, the tokenizer 406B may include any functional process that may be suitable for deconstructing the regulatory sequences 404B or other sequences of textual data (e.g., gene bases “ATGACGGATCAGCCGGCAA” . . . (SEQ ID NO: 1)) into subsets of tokens (e.g., “ATGA”, “CGGA”, “TCAG”, and so forth (e.g., equivalent to deconstructing a sentence into individual phrases or individual words)). In certain embodiments, as further depicted by the workflow diagram 400B of FIG. 4B, the tokenizer 406B may then output a set of tokenized regulatory sequences 408B. In certain embodiments, the workflow diagram 400B may then proceed with performing a token masking process 410B based on the set of tokenized regulatory sequences 408B. For example, in certain embodiments, the token masking process 410B may include any process that may be suitable for performing, for example, a fill-in-the-blank operation (e.g., based on a prediction of missing nucleotides and/or sequences of nucleotides), in which the token masking process 410B may utilize the gene bases surrounding the tokens of the set of tokenized regulatory sequences 408B for predicting the gene base of which the masked token is to be labeled or assigned (e.g., bounded by evolutionary constraints). In one embodiment, the masked tokens 414B may be then utilized as ground truth data for training a pre-trained language ML model 428 that utilizes a sequence of unmasked tokens 412B as input data.

In certain embodiments, the pre-trained language ML model 428 may include, for example, one or more sequence encoder models that may be utilized to predict the sequence content of each individual regulatory sequence subcomponent (promoters, 3′UTR, 5′UTR, CDS, introns, post terminators, and so forth) based on the data set of annotated genome assemblies collected from a targeted gene species 426. For example, in certain embodiments, the pre-trained language ML model 428 may include one or more sequence encoder models that are fine-tuned, for example, in a self-supervised manner on batches of the unmasked tokens 412B as input and the masked tokens 414B as ground truth. The pre-trained language ML model 428 may be then evaluated (e.g., rewarded or penalized) based on its ability to successfully predict gene bases that have been masked in the input sequence of unmasked tokens 412B and then updated the model parameters to minimize the calculated loss of the pre-trained language ML model 428 after each iteration of fine-tuning.

For example, as further depicted by the workflow diagram 400B, the pre-trained language ML model 428 may generate a prediction of masked token vector representations 418B. In certain embodiments, the pre-trained language ML model 428 may generate the prediction of masked token vector representations 418B based on, for example, self-learned grammar, semantics, and syntax of the input sequence of unmasked tokens 412B bounded by evolutionary constraints. For example, due to the evolutionary constraints imposed upon the input sequence of unmasked tokens 412A bounded, the internal state or parameterization of the pre-trained language model 428 may be obliged to approximate the distribution of sequential and evolutionarily-sampled runs of gene base pairs. Under the assumption of independent and identically distributed training data, the approximation may be become increasingly accurate in the large data limit. Additionally, because the pre-trained language ML model 428 may only fit a conditional probability distribution based on the sequence space sampled in the dataset, the dependence of predictions on physical interactions with the environment is implicit.

In certain embodiments, the conditional probability distribution may include a parameterization defined by a learned set of semantic features, which together form the vector representations 418B. In one embodiment, such semantic features may later be interrogated for pertinence to the variation in endophenotype of a gene over various tissues, developmental stages, or in response to a specific stress stimulus. In certain embodiments, the workflow diagram 400B may then proceed with performing a non-linear transformation 420B of the vector representations 418A and generating one or more PMFs of the identities of the masked tokens 422B. For example, in some embodiments, the one or more PMFs of the identities of the masked tokens 422B may represent a function that maps a class label of a respective masked token 422B to the probability of the respective masked token 422B actually taking on that class label. In certain embodiments, the workflow diagram 400B may then proceed with calculating a categorical loss 424B based on the one or more PMFs of the identities of the masked tokens 422B and the ground truth masked tokens 414A. For example, in some embodiments, the categorical loss 424B may include one or more loss functions or cost functions that may be utilized for evaluating how correct or how incorrect a predicted class label for respective unmasked tokens 412A is by comparing against the ground truth masked tokens 414A and then updating (e.g., via backpropagation) the pre-trained language ML model 428 based thereon.

FIG. 4C illustrates an exemplary workflow diagram 400C of a final stage of a model training phase for predicting endophenotype values for targeted genes identified for editing in response to a mutation of one or more cis regulatory sequence gene endophenotypes, in accordance with the presently disclosed embodiments. Specifically, the workflow diagram 400C may be suitable for utilizing a self-supervised sequence encoder model (e.g., fine-tuned language ML model 432 corresponding, in one embodiment, to pre-trained language ML model 428 and randomly-initialized language ML model 416) trained to predict the sequence content of each individual regulatory sequence subcomponent, along with a variant effect predictor model 442 trained to predict the contribution to gene endophenotype (e.g., qualitative biomarker or other measured value) of each individual regulatory sequence subcomponent (promoters, 3′UTR, 5′UTR, CDS, introns, post terminators, and so forth). For example, in certain embodiments, the workflow diagram 400C may begin with obtaining a training data set of regulatory sequence endophenotype pairs 430 that may include, for example, regulatory sequences and their corresponding endophenotype measurements collected from a cell-based assay or plant-based assay.

In some embodiments, the data set of regulatory sequence and endophenotype pairs 430 may be generated via a high-throughput screen, such as RNA-sequencing (RNAseq), microarrays, ribosome profiling, single cell RNASeq, proteome abundance (via two-dimensional gel electrophoresis, mass spectrometry, fluorescent microscopy, etc.), and so forth. In certain embodiments, the promoters and other regulatory sequences included in the data set of regulatory sequence and endophenotype pairs 430 may be filtered by setting a minimum sequence similarity, or, in another embodiment, clusters of similar sequences may be utilized for stratified sampling of training and validation sets. In one embodiment, the data set of regulatory sequence and endophenotype pairs 430 may be provided, for example, to probe the semantic vector space of the masked token vector representations 418A in order to select and weight features salient to the endophenotype in question.

In certain embodiments, the workflow diagram 400C may then proceed with sampling a regulatory sequence 404C and endophenotype 407 pair from the data set of regulatory sequence and endophenotype pairs 430 to be utilized, for example, for further training a fine-tuned language ML model 432. For example, in one embodiment, the data set of regulatory sequences 404C may include a set of sequence and endophenotype measurement pairs. In one embodiment, the endophenotype measurement 407 may include, for example, a class label for a given sequence, a single endophenotype measurement, or an ordered set of measurements for different tissues, developmental stages, growth environments, and so forth.

In certain embodiments, the regulatory sequences 404C may be then inputted to a tokenizer 406C. For example, in certain embodiments, the tokenizer 406C may include any functional process that may be suitable for deconstructing the regulatory sequences 404C or other sequences of textual data (e.g., gene bases “ATGACGGATCAGCCGGCAA” . . . (SEQ ID NO: 1)) into subsets of tokens (e.g., “ATGA”, “CGGA”, “TCAG”, and so forth (e.g., equivalent to deconstructing a sentence into individual phrases or individual words)). In certain embodiments, as further depicted by the workflow diagram 400C of FIG. 4C, the tokenizer 406C may then output a set of tokenized regulatory sequences 408C. In certain embodiments, the workflow diagram 400C may then proceed with inputting the set of tokenized regulatory sequences 408C to the fine-tuned language ML model 432.

In certain embodiments, the fine-tuned language ML model 432 may include, for example, one or more sequence encoder models (e.g. one or more deep neural networks (DNNs)) corresponding, for example, the pre-trained language ML model 428 including a set of predetermined weights. In certain embodiments, the workflow diagram 400C may then proceed with the fine-tuned language ML model 432 generating a set of token vector representations 434. For example, in certain embodiments, the set of token vector representations 434 may include, for example, a set of deep semantic representation vectors for each nucleotide or k-mer in the set of tokenized regulatory sequences 408C. In certain embodiments, the set of token vector representations 434 may be then inputted to a sequence pooling layer 436. In certain embodiments, the sequence pooling layer 436 may include, for example, a randomly-initialized, shallow, and suitably regularized neural network (e.g., convolutional neural network (CNN)) that may be utilized, for example, to reduce (e.g., “pool”) the set of token vector representations 434 to a sequence-specific representation vector 438 by applying a weighted average. Specifically, the sequence pooling layer 436 may be utilized to reduce, for example, the dimensions of the set of token vector representations 434 while retaining the most important information, which is represented by the sequence-specific representation vector 438.

In certain embodiments, over the course of training, the fine-tuned language ML model 432 may learn, for example, a projection of the semantic vector space down a lower-dimensional subspace of features salient to the properties characterized by the training data set of regulatory sequences 404C. For example, in certain embodiments, under the assumption that the regulatory sequence semantic space learned by the fine-tuned language ML model 432 is rich enough to linearly encode a desired endophenotype, the fine-tuned language ML model 432 may be held fixed during training. In such a case, for example, the sequence pooling layer 436 may then be utilized to project from the semantic vector space down to a specific protein property. On the other hand, for example, if the above assumption does not hold, or if the distribution of the desired endophenotype is too subtle to be fully captured by the conditional probability distribution of the fine-tuned language ML model 432, then one or more weights of the fine-tuned language ML model 432 may be allowed to vary during training. This may result, for example, in a non-linear transformation of the regulatory sequence semantic space itself in order to capture more task-specific detail, leading to a more accurate projection down to the desired quantity.

In certain embodiments, the workflow diagram 400C may then proceed with performing a non-linear transformation 440 of the sequence of semantic representations 438. In certain embodiments, the workflow diagram 400C may then proceed with inputting the non-linearly transformed sequence of semantic representations 438 to a variant effect predictor model which may be, for example, a regression or classification model 442. In certain embodiments, the variant effect predictor model 442 may include, for example, any machine-learning model for generating a prediction of an effect score 444 based on the non-linearly transformed sequence of semantic representations 438. For example, in some embodiments, the regression or classification model 442 may include an activation function that outputs an effect score 444 (e.g., predicted endophenotype value) or range of endophenotype values equal to, or proportional to, the range of the endophenotype measurement 407 (e.g., actually measured biomarker value serving as ground truth for training the fine-tuned language ML model 432).

In certain embodiments, the workflow diagram 400C may then proceed with calculating a regression error or categorical loss 446 based on the effect score 444 (e.g., predicted endophenotype value) and the endophenotype measurement 407 (e.g., actually measured biomarker value serving as ground truth for training the fine-tuned language ML model 432). For example, in some embodiments, the regression error or categorical loss 446 may include one or more loss functions or cost functions that may be utilized for evaluating how correct or how incorrect the effect score 444 (e.g., predicted endophenotype value) is by comparing against the ground truth endophenotype measurement 407 and then updating the fine-tuned language ML model 432 based thereon.

In certain embodiments, as previously discussed above with respect to FIGS. 3A and 3B, it should be appreciated that because the trained language ML model 432 captures a conditional probability distribution based on contextual relationships, physical property predictions are sensitive to any mutations in an input sequence. Any such mutation induces a transformation in the learned semantic feature space, and a series of sequence mutations that form a closed loop in this space may represent compensatory mutations and, depending on how the semantic feature space is organized, may be functionally equivalent to the wild-type sequence. The vector representations of input sequences, as given by their learned semantic features, may enable any set of sequence variants to be mapped onto the same high-dimensional vector space. For example, if a sequence is mutated, it may cause a transformation of its vector space representation. Additionally, if the sequence is further mutated, there may be some cases in which the vector representations return to its original position in the vector space. This may form a closed loop in the vector space and may be an indication that the wild-type and final mutated sequences are homologous with respect to the endophenotype measurement 407 since the fine-tuned language ML model 432 represents the wild-type and final mutated sequences as equivalent in their prediction of the effect score 444 (e.g., predicted endophenotype value).

### Predicting Gene Endophenotypes Based on Mutations in Trans Regulatory Factors

### Trans Endophenotype Model Inference Phase

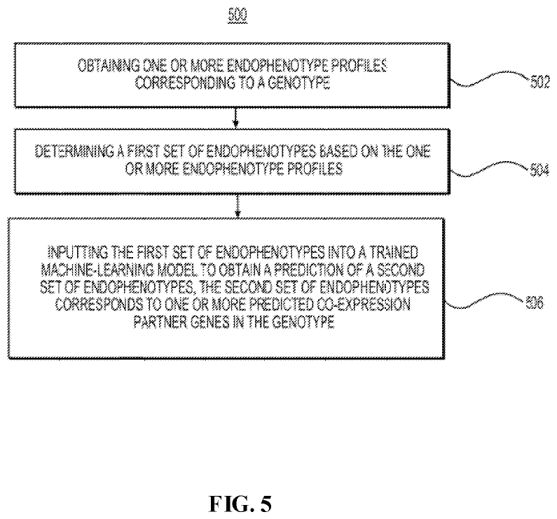

FIG. 5 illustrates a flow diagram 500 for generating in silico predictions of endophenotype values corresponding to one or more interacting genes of a targeted genotype in response to a perturbation of the endophenotype values of one or more trans regulatory factors identified for editing, in accordance with the presently disclosed embodiments. The flow diagram 500 may be performed utilizing one or more processing devices (e.g., genome editing platform 100B) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), or any other processing device(s) that may be suitable for processing genomics data or other omics data), software (e.g., instructions running/executing on one or more processors), firmware (e.g., microcode), or some combination thereof.

The flow diagram 500 may begin at block 502 with one or more processing devices (e.g., genome editing platform 100A) obtaining one or more endophenotype profiles corresponding to a genotype. The flow diagram 500 may then continue at block 504 with one or more processing devices (e.g., genome editing platform 100B) determining a first set of endophenotypes based on the one or more endophenotype profiles. The flow diagram 500 may then conclude at block 506 with one or more processing devices (e.g., genome editing platform 100B) inputting the first set of endophenotypes into a trained machine-learning model to obtain a prediction of a second set of endophenotypes, in which the second set of endophenotypes corresponds to one or more predicted co-expression partner genes in the genotype. As used herein, “partner genes” refers to genes that are co-regulated, co-expressed, or otherwise associated with one another. Partner genes may be part of the same gene regulatory network or pathway. Partner genes may be regulated by one or more transcription factors in common. Partner genes may have direct effects on one other (e.g. the expression of gene 1 increases the expression of gene 2). Partner genes may be positively associated (e.g. increased transcription of gene 3 correlates with increased transcription of gene 4) or negatively associated (e.g. increased transcription of gene 3 correlates with decreased transcription of gene 5).

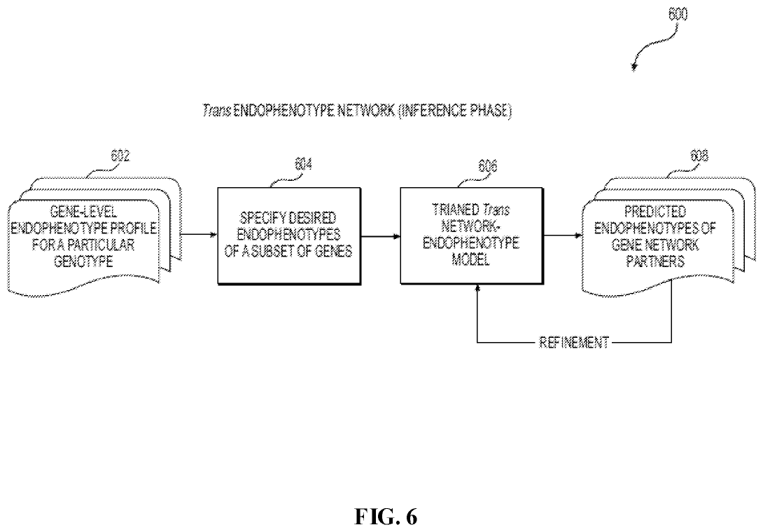

FIG. 6 illustrates an exemplary workflow diagram 600 of an inference phase of a trained model for predicting endophenotype values corresponding to one or more interacting genes of a targeted genotype in response to a perturbation of the endophenotype values of one or more trans regulatory factors identified for editing, in accordance with the presently disclosed embodiments. In certain embodiments, the workflow diagram 600 may begin with obtaining a data set of gene-level endophenotype profiles 602 for one or more targeted genotypes. For example, in certain embodiments, the data set of gene-level endophenotype profiles 602 may include, for example, gene-level endophenotype profiles for the targeted genotype or a subset of interacting genes. In certain embodiments, the workflow diagram 600 may then proceed receiving (e.g., by way of user input) a selection of a subset of the gene-level endophenotypes 604 to be set to desired values. For example, in some embodiments, the subset of gene-level endophenotypes 604 set to the desired values may include, for example, a set of genes targeted for editing in the data set of gene-level endophenotype profiles 602.

In certain embodiments, as further depicted by FIG. 6, the data set of gene-level endophenotype profiles 602 including the subset of gene-level endophenotype profiles 604 set to desired values may be then inputted to one or more trained machine-learning models 606. For example, in certain embodiments, the one or more trained machine-learning models 606 may include, for example, one or more GNN models that may be utilized to output one or more predicted endophenotype values 608 for a subset of interacting genes based on the inputted data set of gene-level endophenotype profiles 602 including the subset of gene-level endophenotype profiles 604 set to desired values. Specifically, the outputted one or more predicted endophenotype values 608 for a subset of interacting genes may include the updated endophenotype values for the subset of interacting genes in response to the subset of gene-level endophenotypes 604 previously set to desired values.

### Trans Endophenotype Model Training Phase

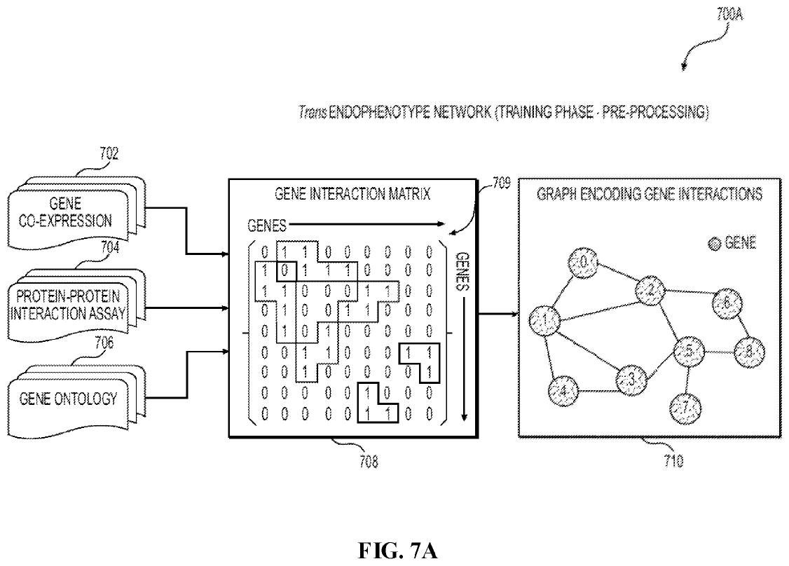

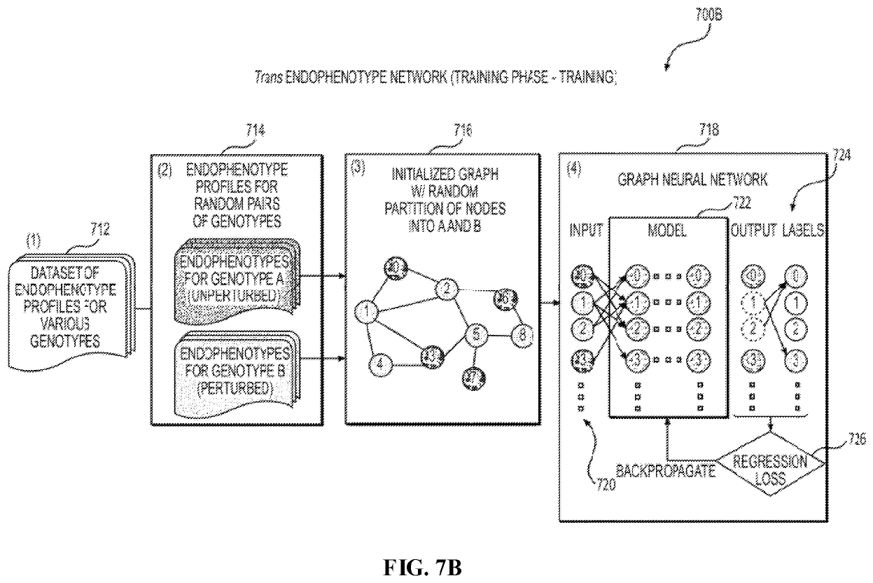

FIG. 7A illustrates an exemplary workflow diagram 700A for a pre-processing stage of a training phase of a model for predicting endophenotype values corresponding to one or more interacting genes of a targeted genotype in response to a perturbation of the endophenotype values of one or more trans regulatory factors identified for editing, in accordance with the presently disclosed embodiments. In certain embodiments, the workflow diagram 700A may begin with obtaining gene co-expression data sets 702, protein-protein interaction assay data sets 704, and gene ontology data sets 706. For example, in certain embodiments, the workflow diagram 700A may include aggregating and incorporating various sources of gene-gene interaction data, protein-protein interaction data (e.g., obtain via chromatin immunoprecipitation sequencing (ChIP-seq)), gene co-expression data, involvement in a given biological process data, sub-cellular localization data, and so forth.

In certain embodiments, the workflow diagram 700A may proceed with inputting the gene co-expression data sets 702, protein-protein interaction assay data sets 704, and gene ontology data sets 706 to a gene-interaction matrix 708. For example, in certain embodiments, the gene-interaction matrix 708 may be utilized to identify one or more pairs of interacting genes 709. In certain embodiments, the one or more pairs of interacting genes 709 may be then utilized to construct a regulatory network graph 710 for an organism of interest. For example, in certain embodiments, nodes of the regulatory network graph 710 may be defined to include, for example, all genes identified in the organism's genome, a subset of the organism's genes that are known to be involved in a certain pathway, a subset of the organism's genes that have non-zero expression in a given tissue or developmental stage of interest, and so forth.

In certain embodiments, edges of the regulatory network graph 710 may be defined to include, for example, edge weights between pairs of nodes. For example, in some embodiments, edge weights may be characterized, for example, by frequency or strength of correlation of pairwise co-expression of measured endophenotypes tied to genes (e.g., gene endophenotype or proteomics) in a suitably large population for a particular tissue, developmental stage, environment, and/or growth conditions in the focal species or in related species; a binary or continuous measure of protein-protein interaction as reported by a suitable experimental assay or predicted by an independently validated ML model; or some combination. In certain embodiments, edges of the regulatory network graph 710 (e.g., a graph pairwise adjacency matrix) may be then constructed based on the nodes of the regulatory network graph 710 and the edges of the regulatory network graph 710.

FIG. 7B illustrates an exemplary workflow diagram 700B for a training stage of a training phase of a model for predicting endophenotype values corresponding to one or more interacting genes of a targeted genotype in response to a perturbation of the endophenotype values of one or more trans regulatory factors identified for editing, in accordance with the presently disclosed embodiments. In certain embodiments, the workflow diagram 700B may begin with obtaining a data set of endophenotype profiles for various genotypes 712. For example, in certain embodiments, to obtain the data set of endophenotype profiles for various genotypes 712 for a given organism of interest, an experiment in which endophenotypes are measured is performed in a manner that may be associated with individual genes.

For example, in some embodiments, the measurement experiments may include, for example, measurements of gene expression, protein expression, or epigenomic state of each gene across a range of individuals or genotypes to generate a quantitative dataset containing raw or normalized counts that are assigned to each gene in the network. In one embodiment, the data set of endophenotype profiles for various genotypes 712 may include endophenotype measurements for any or all of the tissues, developmental stages, environments, and/or growth conditions pertaining to genes in the regulatory network graph 710 as discussed above with respect to FIG. 7A.

In certain embodiments, the workflow diagram 700B may then proceed in randomly assigning pairs of genotypes 714, for which each pair contains a genotype representing an unmodified organism (e.g., “A—Unperturbed”) and a genotype representing a modified organism (e.g., “B—Perturbed”). In certain embodiments, the workflow diagram 700B may then proceed with initializing a graph structure 716 by randomly partitioning the nodes of the graph structure 716 into unperturbed nodes (e.g., “A—Unperturbed”) and perturbed nodes (e.g., “B—Perturbed”). In certain embodiments, the endophenotypes corresponding to the unperturbed genotype are inputted into the unperturbed nodes and the endophenotypes corresponding to the perturbed genotype are inputted into the perturbed nodes. In certain embodiments, as further depicted by FIG. 7B, the workflow diagram 700B may then proceed with providing the graph structure 716 and the node inputs to a graph neural network (GNN) 718.

For example, in certain embodiments, the graph structure 716 (e.g., grouped into input batches 720) may be inputted into one or more GNN models 722. For example, in certain embodiments, the one or more GNN models 722 may include, for example, any machine-learning, graph-based model that may be randomly initialized and trained to predict the endophenotype values (e.g., corresponding to updated endophenotype levels) of the perturbed genotype in the unperturbed nodes given the initial endophenotype values of the unperturbed genotype in the unperturbed nodes as well as the endophenotype values of the perturbed genotype in the perturbed nodes. In certain embodiments, the unperturbed nodes 716 represent specific genes that are unmodified and the perturbed nodes 716 represent specific genes that are mutated either by targeted change to the genome or by untargeted mutagenesis, for example. In certain embodiments, the unperturbed genotype 714 represents the initial genotype of the organism prior to the introduction of any mutations and the perturbed genotype 714 represents the final genotype of the organism after one or more genes have been mutated either by targeted change to the genome or by untargeted mutagenesis, for example. In certain embodiments, the predicted endophenotype values 724 corresponding to the perturbed genotype in the unperturbed nodes represent the final endophenotypic state of the unmodified genes (e.g., unperturbed nodes) due only to interactions in trans with non-overlapping genes (e.g., perturbed nodes) that have been mutated either by targeted change to the genome or by untargeted mutagenesis, for example.

In certain embodiments, the one or more GNN models 722 may then predict a set of co-expression endophenotype values 724 (e.g., corresponding to predicted co-expression partner genes to genes in the regulatory network graph 710 that have been set previously set to a default value and/or perturbed). In certain embodiments, as further depicted by FIG. 7B, the workflow diagram 700B may then proceed with calculating a regression loss 726 based on the predicted set of co-expression endophenotype values 724 and then updating (e.g., via backpropagation) the one or more GNN models 722 model based thereon.

### Predicting Gene Endophenotypes Based on Combination of Cis and Trans Trained Models

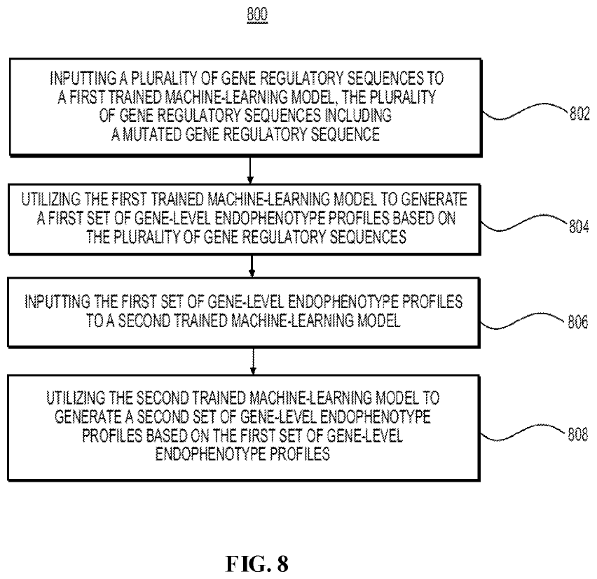

FIG. 8 illustrates a flow diagram 800 for generating an in silico prediction of endophenotype values corresponding to one or more interacting genes in response to mutation of one or more trans regulatory factors identified for editing, in accordance with the presently disclosed embodiments. The flow diagram 800 may be performed utilizing one or more processing devices (e.g., genome editing platform 100A, 100B) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), or any other processing device(s) that may be suitable for processing genomics data or other omics data), software (e.g., instructions running/executing on one or more processors), firmware (e.g., microcode), or some combination thereof.

The flow diagram 800 may begin at block 802 with one or more processing devices (e.g., genome editing platform 100A, 100B) inputting a number of gene regulatory sequences to a first trained machine-learning model, the number of gene regulatory sequences including one or more mutated gene regulatory sequences. The flow diagram 800 may then continue at block 804 with one or more processing devices (e.g., genome editing platform 100A and/or genome editing platform 100B) utilizing the first trained machine-learning model to generate a first set of gene-level endophenotype profiles based on the number of gene regulatory sequences. The flow diagram 800 may then continue at block 806 with one or more processing devices (e.g., genome editing platform 100A) inputting the first set of gene-level endophenotype profiles to a second trained machine-learning model. The flow diagram 800 may then conclude at block 808 with one or more processing devices (e.g., genome editing platform 100A and/or genome editing platform 100B) utilizing the second trained machine-learning model to generate a second set of gene-level endophenotype profiles based on the first set of gene-level endophenotype profiles, in which generating the second set of gene-level endophenotype profiles includes predicting one or more gene-level endophenotype profiles based on one or more mutated gene regulatory sequences.

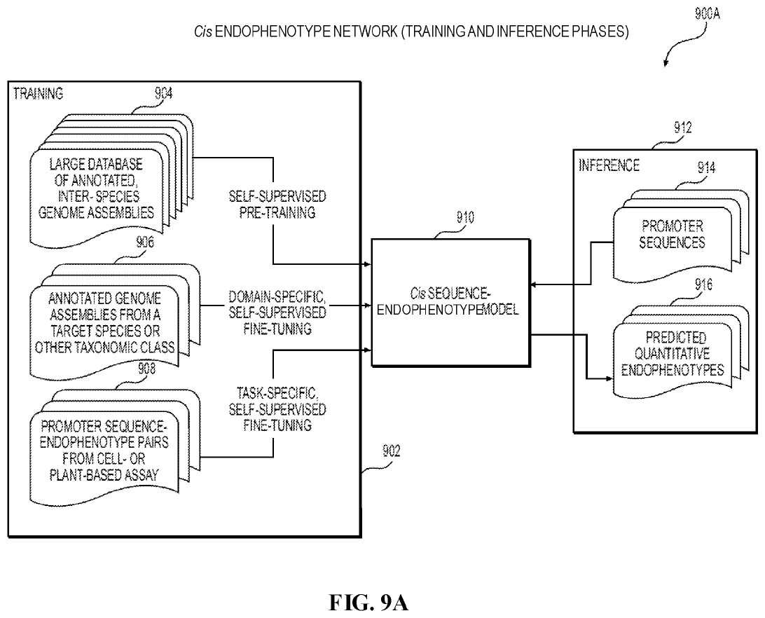

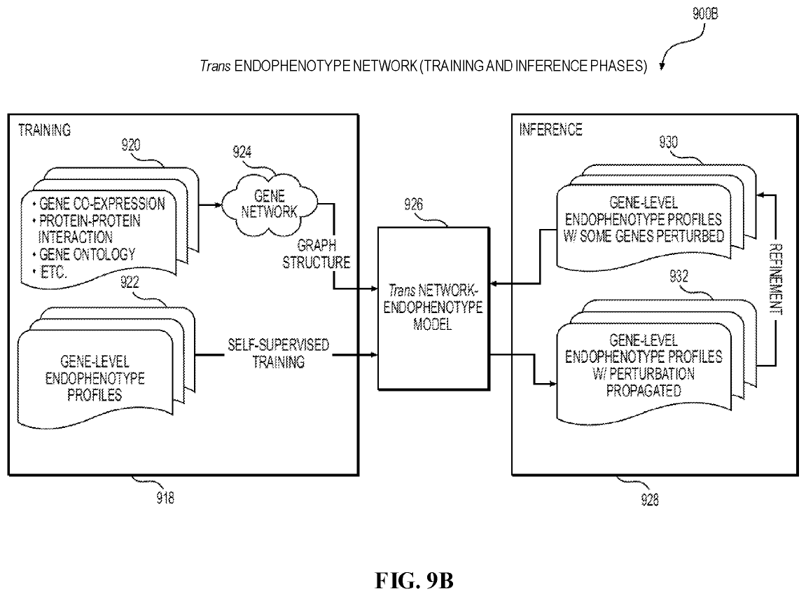

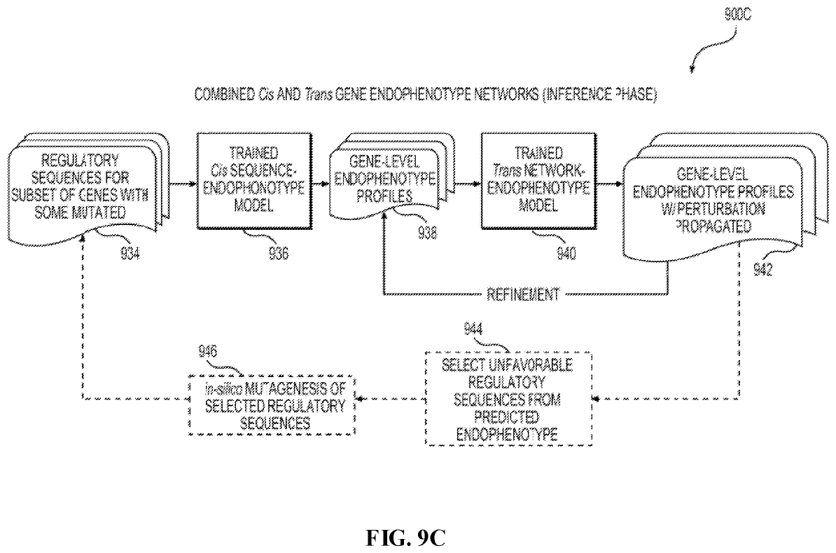

As will be further appreciated with respect to FIGS. 9A, 9B, and 9C below, certain embodiments may combine into a single embodiment the foregoing techniques of generating: 1) an in silico prediction of endophenotype values corresponding to targeted genes in response to a mutation of one or more cis regulatory sequences; and 2) an in silico prediction of endophenotype values corresponding to one or more interacting genes in response to a perturbation of the endophenotype values of one or more targeted trans regulatory factors. For example, in some embodiments, by combining these techniques, the present embodiments (e.g., as discussed below with respect to FIGS. 9A, 9B, and 9C) may be suitable for predicting endophenotype values for targeted genes in response to a mutation of one or more cis regulatory sequences, as well as for predicting the endophenotype values for secondary genes, which are themselves unmodified, but which interact with the targeted genes in trans.

FIG. 9A illustrates an exemplary workflow diagram 900A of a training and inference phase of a cis regulatory sequence to endophenotype effect model, in accordance with the presently disclosed embodiments. In certain embodiments, the workflow diagram 900A may begin with training a cis regulatory sequence to endophenotype model 910 (e.g., corresponding to the one or more trained machine-learning models 310A as discussed above with respect to FIG. 3A) based on a training data set of genome assemblies 902, a training data set of annotated genome assemblies of a target genome 904, a training data set of gene regulatory sequence and endophenotype pairs 906. In certain embodiments, after models are trained, the workflow diagram 900A may proceed with utilizing the cis regulatory sequence to endophenotype model 910 to receive input regulatory sequences 914 and output predicted qualitative or quantitative endophenotype values 916.

FIG. 9B illustrates an exemplary workflow diagram 900B of a training and inference phase of a gene network-based trans endophenotype propagation model, in accordance with the presently disclosed embodiments. In certain embodiments, the workflow diagram 900B may begin with training a trans endophenotype model 926 based on a data set of genomics data 920 constructed into a gene regulatory network 924 and a training data set of gene-level endophenotype profiles 922. In certain embodiments, after models are trained, the workflow diagram 900B may proceed with utilizing the trans endophenotype model 926 (e.g., corresponding to the one or more trained machine-learning models 606 as discussed above with respect to FIG. 6) to receive gene-level endophenotype profiles 930 (e.g., including one or more user-defined perturbed endophenotypes for a subset of genes) and output predicted gene-level endophenotype profiles 932 (e.g., including endophenotype updates propagated indirectly to unmodified genes through interactions in trans).

FIG. 9C illustrates an exemplary workflow diagram 900C of an inference phase for predicting a full gene-level endophenotype profile (e.g., for both modified and unmodified genes) from a gene network and associated regulatory sequences, in accordance with the presently disclosed embodiments. In certain embodiments, the workflow diagram 900C may begin with inputting a data set of gene regulatory sequences 934 to a first trained machine-learning model 936, in which the data set of gene regulatory sequences 934 includes, for example, one or more mutated gene regulatory sequences. In certain embodiments, the workflow diagram 900C may then continue with utilizing the first trained machine-learning model 936 to generate a first set of gene-level endophenotype profiles 938 based on the data set of gene regulatory sequences 934.

In certain embodiments, the workflow diagram 900C may then proceed with inputting the first set of gene-level endophenotype profiles 938 to a second trained machine-learning model 940. In certain embodiments, the workflow diagram 900C may then proceed with inputting the first set of gene-level endophenotype profiles 938 into the second trained machine-learning model 940 to generate a second set of gene-level endophenotype profiles 942 (e.g., in which endophenotypes perturbed from the wild-type state are propagated via the gene network to other genes interacting in trans). For example, in certain embodiments, generating the second set of gene-level endophenotype profiles 942 may include predicting one or more full gene-level endophenotype profiles based on one or more mutated gene regulatory sequences by utilizing, for example, the first trained machine-learning model 936 (e.g., corresponding to the cis sequence endophenotype model 910 discussed above with respect to FIG. 9A) and the second trained machine-learning model 940 (e.g., corresponding to the trans endophenotype model 926 discussed above with respect to FIG. 9B).

### Computing and Artificial Intelligence (AI) Systems Suitable for Predicting Gene Endophenotypes

The machine-learning models described herein may be retrained repeatedly. For example, the system may collect new training data and re-train any of the machine learning models using the new training data. The retrained machine learning model may then be used to provide output based on new input data. Accordingly, the models can be improved iteratively over time as new data is collected.

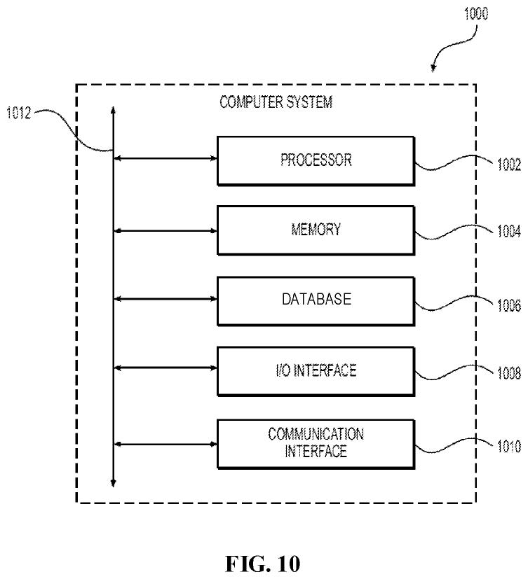

FIG. 10 illustrates an example genome editing computing system 1000 (which may be included as part of the genome editing platform 100A, 100B) that may be utilized for provisioning a platform account and associated sub-account and servicing transactions utilizing the provisioned platform account and associated sub-account, in accordance with the presently disclosed embodiments. In certain embodiments, one or more genome editing computing system 1000 perform one or more steps of one or more methods described or illustrated herein. In certain embodiments, one or more genome editing computing system 1000 provide functionality described or illustrated herein. In certain embodiments, software running on one or more genome editing computing system 1000 performs one or more steps of one or more methods described or illustrated herein or provides functionality described or illustrated herein. Certain embodiments include one or more portions of one or more genome editing computing system 1000. Herein, reference to a computer system may encompass a computing device, and vice versa, where appropriate. Moreover, reference to a computer system may encompass one or more computer systems, where appropriate.

This disclosure contemplates any suitable number of genome editing computing systems 1000. This disclosure contemplates genome editing computing system 1000 taking any suitable physical form. As example and not by way of limitation, genome editing computing system 1000 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, genome editing computing system 1000 may include one or more genome editing computing systems 1000; be unitary or distributed; span multiple locations; span multiple machines; span multiple data centers; or reside in a cloud, which may include one or more cloud components in one or more networks.

Where appropriate, one or more genome editing computing system 1000 may perform without substantial spatial or temporal limitation one or more steps of one or more methods described or illustrated herein. As an example, and not by way of limitation, one or more genome editing computing system 1000 may perform in real time or in batch mode one or more steps of one or more methods described or illustrated herein. One or more genome editing computing system 1000 may perform at different times or at different locations one or more steps of one or more methods described or illustrated herein, where appropriate.

In certain embodiments, genome editing computing system 1000 includes a processor 1002, memory 1004, database 1006, an input/output (I/O) interface 1008, a communication interface 810, and a bus 1012. Although this disclosure describes and illustrates a particular computer system having a particular number of particular components in a particular arrangement, this disclosure contemplates any suitable computer system having any suitable number of any suitable components in any suitable arrangement. In certain embodiments, processor 1002 includes hardware for executing instructions, such as those making up a computer program. As an example, and not by way of limitation, to execute instructions, processor 1002 may retrieve (or fetch) the instructions from an internal register, an internal cache, memory 1004, or database 1006; decode and execute them; and then write one or more results to an internal register, an internal cache, memory 1004, or database 1006. In certain embodiments, processor 1002 may include one or more internal caches for data, instructions, or addresses. This disclosure contemplates processor 1002 including any suitable number of any suitable internal caches, where appropriate. As an example, and not by way of limitation, processor 1002 may include one or more instruction caches, one or more data caches, and one or more translation lookaside buffers (TLBs). Instructions in the instruction caches may be copies of instructions in memory 1004 or database 1006, and the instruction caches may speed up retrieval of those instructions by processor 1002.

Data in the data caches may be copies of data in memory 1004 or database 1006 for instructions executing at processor 1002 to operate on; the results of previous instructions executed at processor 1002 for access by subsequent instructions executing at processor 1002 or for writing to memory 1004 or database 1006; or other suitable data. The data caches may speed up read or write operations by processor 1002. The TLBs may speed up virtual-address translation for processor 1002. In certain embodiments, processor 1002 may include one or more internal registers for data, instructions, or addresses. This disclosure contemplates processor 1002 including any suitable number of any suitable internal registers, where appropriate. Where appropriate, processor 1002 may include one or more arithmetic logic units (ALUs); be a multi-core processor; or include one or more processors 802. Although this disclosure describes and illustrates a particular processor, this disclosure contemplates any suitable processor.

In certain embodiments, memory 1004 includes main memory for storing instructions for processor 1002 to execute or data for processor 1002 to operate on. As an example, and not by way of limitation, genome editing computing system 1000 may load instructions from database 1006 or another source (such as, for example, another genome editing computing system 1000) to memory 1004. Processor 1002 may then load the instructions from memory 1004 to an internal register or internal cache. To execute the instructions, processor 1002 may retrieve the instructions from the internal register or internal cache and decode them. During or after execution of the instructions, processor 1002 may write one or more results (which may be intermediate or final results) to the internal register or internal cache. Processor 1002 may then write one or more of those results to memory 1004. In certain embodiments, processor 1002 executes only instructions in one or more internal registers or internal caches or in memory 1004 (as opposed to database 1006 or elsewhere) and operates only on data in one or more internal registers or internal caches or in memory 1004 (as opposed to database 1006 or elsewhere).

One or more memory buses (which may each include an address bus and a data bus) may couple processor 1002 to memory 1004. Bus 1012 may include one or more memory buses, as described below. In certain embodiments, one or more memory management units (MMUs) reside between processor 1002 and memory 1004 and facilitate accesses to memory 1004 requested by processor 1002. In certain embodiments, memory 1004 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1004 may include one or more memory devices 1004, where appropriate. Although this disclosure describes and illustrates particular memory, this disclosure contemplates any suitable memory.

In certain embodiments, database 1006 includes mass storage for data or instructions. As an example, and not by way of limitation, database 1006 may include a hard disk drive (HDD), a floppy disk drive, flash memory, an optical disc, a magneto-optical disc, magnetic tape, or a Universal Serial Bus (USB) drive or a combination of two or more of these. Database 1006 may include removable or non-removable (or fixed) media, where appropriate. Database 1006 may be internal or external to genome editing computing system 1000, where appropriate. In certain embodiments, database 1006 is non-volatile, solid-state memory. In certain embodiments, database 1006 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass database 1006 taking any suitable physical form. Database 1006 may include one or more storage control units facilitating communication between processor 1002 and database 1006, where appropriate. Where appropriate, database 1006 may include one or more storages 1006. Although this disclosure describes and illustrates particular storage, this disclosure contemplates any suitable storage.

In certain embodiments, I/O interface 1008 includes hardware, software, or both, providing one or more interfaces for communication between genome editing computing system 1000 and one or more I/O devices. Genome editing computing system 1000 may include one or more of these I/O devices, where appropriate. One or more of these I/O devices may enable communication between a person and genome editing computing system 1000. As an example, and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1006 for them. Where appropriate, I/O interface 1008 may include one or more device or software drivers enabling processor 1002 to drive one or more of these I/O devices. I/O interface 1008 may include one or more I/O interfaces 1006, where appropriate. Although this disclosure describes and illustrates a particular I/O interface, this disclosure contemplates any suitable I/O interface.

In certain embodiments, communication interface 810 includes hardware, software, or both providing one or more interfaces for communication (such as, for example, packet-based communication) between genome editing computing system 1000 and one or more other computer systems 1000 or one or more networks. As an example, and not by way of limitation, communication interface 810 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 810 for it.

As an example, and not by way of limitation, genome editing computing system 1000 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, genome editing computing system 1000 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Genome editing computing system 1000 may include any suitable communication interface 810 for any of these networks, where appropriate. Communication interface 810 may include one or more communication interfaces 810, where appropriate. Although this disclosure describes and illustrates a particular communication interface, this disclosure contemplates any suitable communication interface.

In certain embodiments, bus 1012 includes hardware, software, or both coupling components of genome editing computing system 1000 to each other. As an example, and not by way of limitation, bus 1012 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1012 may include one or more buses 1012, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

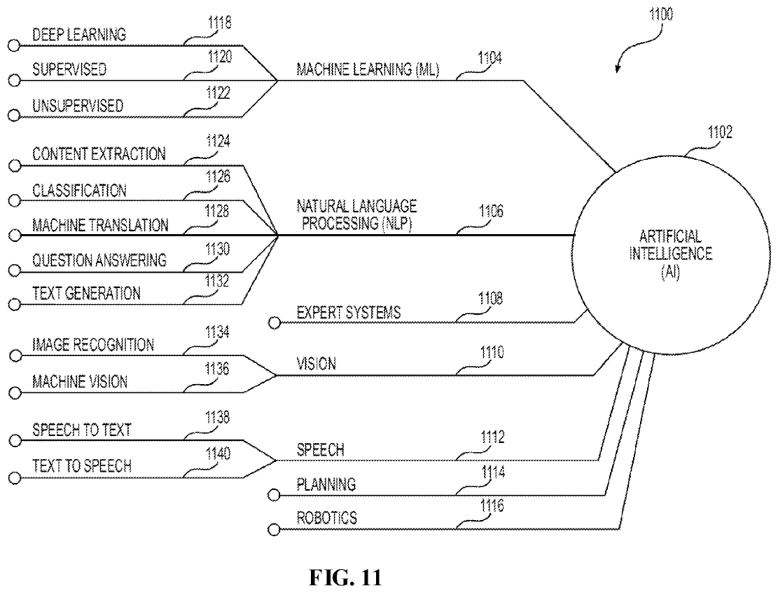

FIG. 11 illustrates a diagram 1100 of an example artificial intelligence (AI) architecture 1102 (which may be included as part of the genome editing platform 100A and/or genome editing platform 100B) that may be utilized for provisioning a platform account and associated sub-account and servicing transactions utilizing the provisioned platform account and associated sub-account, in accordance with the presently disclosed embodiments. In certain embodiments, the AI architecture 1102 may be implemented utilizing, for example, one or more processing devices that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), and/or other processing device(s) that may be suitable for processing various data and making one or more decisions based thereon), software (e.g., instructions running/executing on one or more processing devices), firmware (e.g., microcode), or some combination thereof.

In certain embodiments, as depicted by FIG. 11, the AI architecture 1102 may include machine learning (ML) algorithms and functions 1104, natural language processing (NLP) algorithms and functions 1106, expert systems 1108, computer-based vision algorithms and functions 1110, speech recognition algorithms and functions 1112, planning algorithms and functions 1114, and robotics algorithms and functions 1116. In certain embodiments, the ML algorithms and functions 1104 may include any statistics-based algorithms that may be suitable for finding patterns across large amounts of data (e.g., “Big Data” such as genomics data, proteomics data, metabolomics data, metagenomics data, and transcriptomics data, or other omics data). For example, in certain embodiments, the ML algorithms and functions 1104 may include deep learning algorithms 1118, supervised learning algorithms 1120, and unsupervised learning algorithms 1122.

In certain embodiments, the deep learning algorithms 1118 may include any artificial neural networks (ANNs) that may be utilized to learn deep levels of representations and abstractions from large amounts of data. For example, the deep learning algorithms 1118 may include ANNs, such as a multilayer perceptron (MLP), an autoencoder (AE), a convolution neural network (CNN), a recurrent neural network (RNN), long short term memory (LSTM), a gated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), a spiking neural network (SNN), deep reinforcement learning, and so forth.

In certain embodiments, the supervised learning algorithms 1120 may include any algorithms that may be utilized to apply, for example, what has been learned in the past to new data using labeled examples for predicting future events. For example, starting from the analysis of a known training data set, the supervised learning algorithms 1120 may produce an inferred function to make predictions about the output values. The supervised learning algorithms 620 can also compare its output with the correct and intended output and find errors in order to modify the supervised learning algorithms 1120 accordingly. On the other hand, the unsupervised learning algorithms 1122 may include any algorithms that may applied, for example, when the data used to train the unsupervised learning algorithms 1122 are neither classified nor labeled. For example, the unsupervised learning algorithms 1122 may study and analyze how systems may infer a function to describe a hidden structure from unlabeled data.

In certain embodiments, the NLP algorithms and functions 1106 may include any algorithms or functions that may be suitable for automatically manipulating natural language, such as speech and/or text. For example, in some embodiments, the NLP algorithms and functions 1106 may include content extraction algorithms or functions 1124, classification algorithms or functions 1126, machine translation algorithms or functions 1128, question answering (QA) algorithms or functions 1130, and text generation algorithms or functions 1132. In certain embodiments, the content extraction algorithms or functions 1124 may include a means for extracting text or images from electronic documents (e.g., webpages, text editor documents, and so forth) to be utilized, for example, in other applications.

In certain embodiments, the classification algorithms or functions 1126 may include any algorithms that may utilize a supervised learning model (e.g., logistic regression, naïve Bayes, stochastic gradient descent (SGD), k-nearest neighbors, decision trees, random forests, support vector machine (SVM), and so forth) to learn from the data input to the supervised learning model and to make new observations or classifications based thereon. The machine translation algorithms or functions 1128 may include any algorithms or functions that may be suitable for automatically converting source text in one language, for example, into text in another language. Indeed, in certain embodiments, the machine translation algorithms or functions 728 may be suitable for performing any of various language translation, text string-based translation, or textual representation translation applications. The QA algorithms or functions 1130 may include any algorithms or functions that may be suitable for automatically answering questions posed by humans in, for example, a natural language, such as that performed by voice-controlled personal assistant devices. The text generation algorithms or functions 1132 may include any algorithms or functions that may be suitable for automatically generating natural language texts.

In certain embodiments, the expert systems 1108 may include any algorithms or functions that may be suitable for simulating the judgment and behavior of a human or an organization that has expert knowledge and experience in a particular field (e.g., stock trading, medicine, sports statistics, and so forth). The computer-based vision algorithms and functions 1110 may include any algorithms or functions that may be suitable for automatically extracting information from images (e.g., photo images, video images). For example, the computer-based vision algorithms and functions 1110 may include image recognition algorithms 1134 and machine vision algorithms 1136. The image recognition algorithms 1134 may include any algorithms that may be suitable for automatically identifying and/or classifying objects, places, people, and so forth that may be included in, for example, one or more image frames or other displayed data. The machine vision algorithms 1136 may include any algorithms that may be suitable for allowing computers to “see”, or, for example, to rely on image sensors cameras with specialized optics to acquire images for processing, analyzing, and/or measuring various data characteristics for decision making purposes.

In certain embodiments, the speech recognition algorithms and functions 1112 may include any algorithms or functions that may be suitable for recognizing and translating spoken language into text, such as through automatic speech recognition (ASR), computer speech recognition, speech-to-text (STT), or text-to-speech (TTS) in order for the computing to communicate via speech with one or more users, for example. In certain embodiments, the planning algorithms and functions 1114 may include any algorithms or functions that may be suitable for generating a sequence of actions, in which each action may include its own set of preconditions to be satisfied before performing the action. Examples of AI planning may include classical planning, reduction to other problems, temporal planning, probabilistic planning, preference-based planning, conditional planning, and so forth. Lastly, the robotics algorithms and functions 616 may include any algorithms, functions, or systems that may enable one or more devices to replicate human behavior through, for example, motions, gestures, performance tasks, decision-making, emotions, and so forth.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

The embodiments disclosed herein are only examples, and the scope of this disclosure is not limited to them. Embodiments according to this disclosure are in particular disclosed in the attached claims directed to a method, a storage medium, a system and a computer program product, wherein any feature mentioned in one claim category, e.g. method, can be claimed in another claim category, e.g. system, as well. The dependencies or references back in the attached claims are chosen for formal reasons only. However, any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates certain embodiments as providing particular advantages, certain embodiments may provide none, some, or all of these advantages.

### Embodiments

1A. A method of modifying an endophenotype in a plant, the method comprising, by one or more computing devices: obtaining a plurality of gene regulatory sequences; inputting the plurality of gene regulatory sequences into a machine-learning model trained to obtain a plurality of effect predictions corresponding to a plurality of endophenotypes; selecting one or more desired endophenotypes based on the plurality of endophenotypes; selecting a gene regulatory sequence in accordance with the one or more desired endophenotypes, and introducing the selected gene regulatory sequence into the plant, thereby modifying the endophenotype of the plant.
2A. A method for generating a gene regulatory sequence with a desired endophenotype profile, the method comprising, by one or more computing devices: obtaining a plurality of gene regulatory sequences; inputting the plurality of gene regulatory sequences into a machine-learning model trained to obtain a plurality of effect predictions corresponding to a plurality of endophenotypes; selecting one or more desired endophenotypes based on the plurality of endophenotypes; and selecting a gene regulatory sequence in accordance with the one or more desired endophenotypes.
3A. The method of any one of embodiments 1A or 2A, wherein selecting the gene regulatory sequence comprises selecting a gene regulatory sequence in accordance with a desired endophenotype level.
4A. The method of embodiment 3A, wherein the desired endophenotype level comprises a desired messenger RNA (mRNA) expression level.
5A. The method of any one of embodiments 1A-4A, wherein the one or more computing devices are associated with a genome editing platform, the genome editing platform configured to generate the gene regulatory sequence with the desired endophenotype profile.
6A. The method of any one of embodiments 1A-5A, wherein obtaining the plurality of gene regulatory sequences comprises: obtaining a plurality of gene promoter regulatory sequences, a plurality of gene terminator regulatory sequences, a plurality of gene enhancer regulatory sequences, a plurality of gene repressor regulatory sequences, a plurality of transcription factor binding sites, and/or a plurality of synthetic gene regulatory sequences.
7A. The method of any one of embodiments 1A-6A, wherein the machine-learning model comprises one or more sequence encoder models.
8A. The method of any one of embodiments 1A-7A, wherein the machine-learning model is trained by: pre-training a randomly-initialized sequence encoder model utilizing a self-supervised prediction of the one or more gene regulatory sequences; and fine-tuning the pre-trained sequence encoder model utilizing a self-supervised prediction of a plurality of gene regulatory sequences extracted from a targeted taxonomic unit.
9A. The method of embodiment 8A, wherein the machine-learning model is trained further by: utilizing a variant effect predictor model with inputs generated by the sequence encoder model to: 1) further fine-tune the weights of the sequence encoder model and 2) generate effect predictions corresponding to a plurality of candidate endophenotypes of interest.
10A. The method of embodiment 9A, wherein the machine-learning model is trained further by: computing a loss value based on a comparison of the effect predictions and an endophenotype measurement; and training the variant effect predictor model based on a backpropagation of the computed loss value.
11A. The method of embodiment 10A, further comprising utilizing the variant effect predictor model to predict a particular endophenotype measurement observed from one or more cell-based assays or one or more plant-based assays.
12A. The method of any one of embodiments 1A-11A, wherein the machine-learning model comprises one or more sequence space-sampling algorithms.
13A. The method of embodiment 12A, further comprising: subsequent to obtaining the plurality of gene regulatory sequences: inputting a plurality of seed gene regulatory sequences into the one or more sequence space-sampling algorithms; and obtaining the plurality of effect predictions by: 1) computationally sampling the space of gene regulatory sequences, and 2) inputting the plurality of sampled gene regulatory sequences into the one or more trained machine-learning models to obtain the plurality of effect predictions corresponding to the plurality of endophenotypes.
14A. The method of embodiment 13A, wherein the one or more sequence space-sampling algorithms comprise one or more generative adversarial networks (GANs), one or more variational autoencoders (VAEs), or one or more Markov chain Monte Carlo (MCMC) sampling algorithms.
15A. The method of any one of embodiments 13A or 14A, wherein obtaining the plurality of effect predictions corresponding to the plurality of endophenotypes comprises iteratively providing as feedback a plurality of sampled gene regulatory sequences as seed sequences for the one or more sequence space-sampling algorithms until the one or more desired endophenotypes are produced.
16A. The method of any one of embodiments 1A-15A, wherein the selected gene regulatory sequence is operably linked to an exogenous or endogenous transcript, and is provided in a vector for expressing the exogenous or endogenous transcript.
17A. The method of any one of embodiments 1A-16A, further comprising generating a donor template nucleic acid comprising the gene regulatory sequence or a portion thereof.
18A. The method of any one of embodiments 1A-17A, further comprising generating one or more guide RNAs (gRNAs) targeting a genomic location to promote introduction of the gene regulatory sequence.
19A. The method of any one of embodiments 17A or 18A, wherein the guide RNA and/or donor template nucleic acid is configured to introduce a selected modified gene regulatory sequence into one or more plants.
20A. The method of any one of embodiments 1A-19A, wherein the machine-learning model was trained utilizing data representing information related to RNAseq, microarrays, ribosome profiling, single cell RNASeq, and/or proteome abundance.
21A. The method of any one of embodiments 1A-20A, wherein the one or more desired endophenotypes comprises a tissue-specific gene endophenotype, a temporally-controlled gene endophenotype, or a change in gene endophenotype in response to a stimulus.
22A. The method of any one of embodiments 1A-21A, further comprising introducing the selected gene regulatory sequence into a plant.
23A. A plant comprising a modified gene regulatory sequence generated by the method of embodiment 22A.
24A. A system including one or more computing devices, comprising: one or more non-transitory computer-readable storage media including instructions; and one or more processors coupled to the one or more storage media, the one or more processors configured to execute the instructions to: obtain a plurality of gene regulatory sequences; input the plurality of gene regulatory sequences into a machine-learning model trained to obtain a plurality of effect predictions corresponding to a plurality of endophenotypes; select one or more desired endophenotypes based on the plurality of endophenotypes; and select a gene regulatory sequence in accordance with the one or more desired endophenotypes.
25A. The system of embodiment 24A, wherein the one or more desired endophenotypes comprise a desired messenger RNA (mRNA) expression level.
26A. The system of any one of embodiments 24A-25A, wherein the one or more computing devices are associated with a genome editing platform, the genome editing platform configured to generate the gene regulatory sequence with the desired endophenotype profile.
27A. The system of any one of embodiments 24A-26A, wherein the instructions to obtain the plurality of gene regulatory sequences further comprise instructions to obtain a plurality of gene promoter regulatory sequences, a plurality of gene terminator regulatory sequences, a plurality of gene enhancer regulatory sequences, a plurality of gene repressor regulatory sequences, a plurality of transcription factor binding sites, and/or a plurality of synthetic gene regulatory sequences.
28A. The system of any one of embodiments 24A-27A, wherein the machine-learning model comprises one or more sequence encoder models.
29A. The system of embodiment 28A, wherein the machine-learning model is trained by: pre-training a randomly-initialized sequence encoder model utilizing a self-supervised prediction of the one or more gene regulatory sequences; and fine-tuning the pre-trained sequence encoder model utilizing a self-supervised prediction of a plurality of gene regulatory sequences extracted from a targeted taxonomic unit.
30A. The system of embodiment 29A, wherein the machine-learning model is trained further by:
utilizing a variant effect predictor model with inputs generated by the sequence encoder model to: 1) further fine-tune the weights of the sequence encoder model and 2) generate effect predictions corresponding to a plurality of candidate endophenotypes of interest.
31A. The system of embodiment 30A, wherein the machine-learning model is trained further by:
computing a loss value based on a comparison of the effect predictions and an endophenotype measurement; and training the variant effect predictor model based on a backpropagation of the computed loss value.
32A. The system of embodiment 31A, wherein the instructions further comprise instructions to utilize the variant effect predictor model to predict a particular endophenotype measurement observed from one or more cell-based assays or one or more plant-based assays.
33A. The system of embodiment 24A-32A, wherein the machine-learning model comprises one or more sequence space-sampling algorithms.
34A. The system of embodiment 33A, wherein the instructions further comprise instructions to: subsequent to obtaining the plurality of gene regulatory sequences: input a plurality of seed gene regulatory sequences into the one or more sequence space-sampling algorithms; and obtain the plurality of effect predictions by: 1) computationally sampling the space of gene regulatory sequences, and 2) inputting the plurality of sampled gene regulatory sequences into the one or more trained machine-learning models to obtain the plurality of effect predictions corresponding to the plurality of endophenotypes.
35A. The system of any one of embodiments 33A or 34A, wherein the one or more sequence space-sampling algorithms comprise one or more generative adversarial networks (GANs), one or more variational autoencoders (VAEs), or one or more Markov chain Monte Carlo (MCMC) sampling algorithms.
36A. The system of any one of embodiments 33A-35A, wherein the instructions to obtain the plurality of effect predictions corresponding to the plurality of endophenotypes further comprise instructions to iteratively provide as feedback a plurality of sampled gene regulatory sequences as seed sequences for the one or more sequence space-sampling algorithms until the one or more desired endophenotypes are produced.
37A. The system of any one of embodiments 24A-36A, wherein the selected gene regulatory sequence is operably linked to an exogenous or endogenous transcript, and is provided in a vector for expressing an exogenous or endogenous transcript.
38A. The system of any one of embodiments 24A-37A, wherein the instructions further comprise instructions to generate a donor template nucleic acid comprising the gene regulatory sequence or a portion thereof.
39A. The system of any one of embodiments 24A-38A, wherein the instructions further comprise instructions to generate one or more guide RNAs (gRNAs) targeting a genomic location to promote introduction of the gene regulatory sequence.
40A. The system of any one of embodiments 38A-39A, wherein the guide RNA and/or donor template nucleic acid is configured to introduce a selected modified gene regulatory sequence into one or more plants.
41A. The system of any one of embodiments 24A-40A, wherein the machine-learning model was trained utilizing data representing information related to RNAseq, microarrays, ribosome profiling, single cell RNASeq, and/or proteome abundance.
42A. The system of any one of embodiments 24A-41A, wherein the one or more desired endophenotypes comprises a tissue-specific gene endophenotype, a temporally-controlled gene endophenotype, or a change in gene endophenotype in response to a stimulus.
43A. A non-transitory computer-readable medium comprising instructions that, when executed by one or more processors of one or more computing devices, cause the one or more processors to: obtain a plurality of gene regulatory sequences; input the plurality of gene regulatory sequences into a machine-learning model trained to obtain a plurality of effect predictions corresponding to a plurality of endophenotypes; select one or more desired endophenotypes based on the plurality of endophenotypes; and select a gene regulatory sequence in accordance with the one or more desired endophenotypes.
44A. The non-transitory computer-readable medium of embodiment 43A, wherein the desired endophenotype level comprises a desired messenger RNA (mRNA) expression level.
45A. The non-transitory computer-readable medium of any one of embodiments 43A or 44A, wherein the one or more computing devices are associated with a genome editing platform, the genome editing platform configured to generate the gene regulatory sequence with the desired endophenotype profile.
46A. The non-transitory computer-readable medium of any one of embodiments 43A-45A, wherein the instructions to obtain the plurality of gene regulatory sequences further comprise instructions to obtain a plurality of gene promoter regulatory sequences, a plurality of gene terminator regulatory sequences, a plurality of gene enhancer regulatory sequences, a plurality of gene repressor regulatory sequences, a plurality of transcription factor binding sites, and/or a plurality of synthetic gene regulatory sequences.
47A. The non-transitory computer-readable medium of embodiments 43A-46A, wherein the machine-learning model comprises one or more sequence encoder models.
48A. The non-transitory computer-readable medium of embodiments 43A-47A, wherein the machine-learning model is trained by: pre-training a randomly-initialized sequence encoder model utilizing a self-supervised prediction of the one or more gene regulatory sequences; and fine-tuning the pre-trained sequence encoder model utilizing a self-supervised prediction of a plurality of gene regulatory sequences extracted from a targeted taxonomic unit.
49A. The non-transitory computer-readable medium of embodiment 48A, wherein the machine-learning model is trained further by: utilizing a variant effect predictor model with inputs generated by the sequence encoder model to: 1) further fine-tune the weights of the sequence encoder model and 2) generate effect predictions corresponding to a plurality of candidate endophenotypes of interest.
50A. The non-transitory computer-readable medium of embodiment 49A, wherein the machine-learning model is trained further by: computing a loss value based on a comparison of the effect predictions and an endophenotype measurement; and training the variant effect predictor model based on a backpropagation of the computed loss value.
51A. The non-transitory computer-readable medium of embodiment 50A, wherein the instructions further comprise instructions to utilize the variant effect predictor model to predict a particular endophenotype measurement observed from one or more cell-based assays or one or more plant-based assays.
52A. The non-transitory computer-readable medium of any one of embodiments 43A-51A, wherein the machine-learning model comprises one or more sequence space-sampling algorithms.
53A. The non-transitory computer-readable medium of embodiment 52A, wherein the instructions further comprise instructions to: subsequent to obtaining the plurality of gene regulatory sequences: input a plurality of seed gene regulatory sequences into the one or more sequence space-sampling algorithms; and obtain the plurality of effect predictions by: 1) computationally sampling the space of gene regulatory sequences, and 2) inputting the plurality of sampled gene regulatory sequences into the one or more trained machine-learning models to obtain the plurality of effect predictions corresponding to the plurality of endophenotypes.
54A. A method for predicting the effect of a mutated gene regulatory sequence the method comprising, by one or more computing devices: inputting a plurality of gene regulatory sequences to a first trained machine-learning model, the plurality of gene regulatory sequences comprising one or more mutated gene regulatory sequences; utilizing the first trained machine-learning model to generate a first set of gene-level endophenotype profiles based on the plurality of gene regulatory sequences, comprising cis regulatory effects of the one or more mutated gene regulatory sequences; inputting the first set of gene-level endophenotype profiles to a second trained machine-learning model; and utilizing the second trained machine-learning model to generate a second set of gene-level endophenotype profiles based on the first set of gene-level endophenotype profiles, wherein generating the second set of gene-level endophenotype profiles comprises predicting one or more updated gene-level endophenotype profiles based on the plurality of gene regulatory sequences including the trans regulatory effects of the one or more mutated gene regulatory sequences.
55A. The method of embodiment 54A, wherein the one or more computing devices are associated with a genome editing platform, and wherein the genome editing platform is configured to assess the effect of the one or more mutated gene regulatory sequences on all genes in the genome or pathway due to both cis and trans regulatory effects.
56A. The method of any one of embodiments 54A-55A, further comprising providing as feedback the predicted second set of gene-level endophenotype profiles to the second trained machine-learning model.
57A. The method of embodiment 56A, wherein providing as feedback the predicted second set of gene-level endophenotype profiles to the second trained machine-learning model comprises refining the prediction of the second set of gene-level endophenotype profiles in accordance with a predetermined evaluation metric.
58A. The method of any one of embodiments 54A-57A, wherein the first trained machine-learning model comprises one or more sequence encoder models including language-based models adapted from natural language processing (NLP) and one or more variant effect predictor models including classification or regression models
59A. The method of any one of embodiments 54A-58A, further comprising: training the first trained machine-learning model by: pre-training a randomly-initialized language model utilizing a self-supervised prediction of one or more gene regulatory sequences extracted from a wide variety of species; and fine-tuning the pre-trained language model utilizing a self-supervised prediction of a plurality of gene regulatory sequences extracted from a targeted species.
60A. The method of embodiments 59A, wherein training the first machine-learning model further comprises: training a regression or classification model with input features generated by the fine-tuned language model to generate effect predictions corresponding to a plurality of candidate endophenotypes of interest.
61A. The method of embodiments 60A, further comprising utilizing the regression or classification model to predict a particular endophenotype measurement observed from one or more cell-based assays or one or more plant-based assays.
62A. The method of embodiment 61A, further comprising: observing the particular endophenotype measurement from the one or more cell-based assays or one or more plant-based assays; and training the regression or classification model by: computing a loss value based on a comparison of the effect predictions and the endophenotype measurement; and training the regression or classification model based on a backpropagation of the computed loss value.
63A. The method of any one of embodiments 54A-62A, wherein the second trained machine-learning model comprises one or more graph neural networks (GNNs).
64A. The method of embodiment 63A, further comprising: training the second machine-learning model by: aggregating a dataset of endophenotype profiles of various genotypes corresponding to a target organism or pathway; and selecting one or more random pairs of genotypes from the dataset of endophenotype profiles of genotypes for which each pair represents an unmodified and modified organism respectively.
65A. The method of embodiment 64A, wherein training the second machine-learning model further comprises:
initializing the one or more GNNs by randomly partitioning nodes of graphs comprising the one or more GNNs into a first set of nodes corresponding to a first genotype of the one or more random pairs of genotypes and a second set of nodes corresponding to a second genotype of the one or more random pairs of genotypes.
66A. The method of embodiment 65A, wherein training the second machine-learning model further comprises: training the one or more GNNs to predict the endophenotypes corresponding to first set of nodes for the second genotype given both the endophenotypes corresponding to the first set of nodes for the first genotype and the endophenotypes corresponding to the second set of nodes for the second genotype.
67A. The method of any one of embodiments 54A-66A, wherein the second set of gene-level endophenotype profiles is predicted for a modified genotype of one or more plant seeds.
68A. The method of any one of embodiments 54A-67A, wherein the first trained machine-learning model and the second trained machine-learning model were trained utilizing data representing information related to RNAseq, microarrays, ribosome profiling, single cell RNASeq, and/or proteome abundance.
69A. The method of any one of embodiments 54A-68A, wherein the first set of gene-level endophenotype profiles comprises a tissue-specific gene endophenotype, a temporally-controlled gene endophenotype, or a change in gene endophenotype in response to a stimulus.
70A. The method of any one of embodiments 54A-69A, further comprising introducing a mutated gene regulatory sequence to a plant based on the one or more predicted gene-level endophenotype profiles.
71A. A plant comprising a mutated gene regulatory sequence and/or predicted gene-level endophenotype profiles generated by the method of embodiment 70A.
72A. A system including one or more computing devices, comprising: one or more non-transitory computer-readable storage media including instructions; and one or more processors coupled to the one or more storage media, the one or more processors configured to execute the instructions to: input a plurality of gene regulatory sequences to a first trained machine-learning model, the plurality of gene regulatory sequences comprising one or more mutated gene regulatory sequences; utilize the first trained machine-learning model to generate a first set of gene-level endophenotype profiles based on the plurality of gene regulatory sequences, including the cis regulatory effects of the one or more mutated gene regulatory sequences; input the first set of gene-level endophenotype profiles to a second trained machine-learning model; and utilize the second trained machine-learning model to generate a second set of gene-level endophenotype profiles based on the first set of gene-level endophenotype profiles, wherein generating the second set of gene-level endophenotype profiles comprises predicting one or more updated gene-level endophenotype profiles based on the plurality of gene regulatory sequences including the trans regulatory effects of the one or more mutated gene regulatory sequences.
73A. The system of embodiment 72A, wherein the one or more computing devices are associated with a genome editing platform, and wherein the genome editing platform is configured to assess the effect of the one or more mutated gene regulatory sequences on all genes in the genome or pathway due to both cis and trans regulatory effects.
74A. The system of any one of embodiments 72A-73A, wherein the instructions further comprise instructions to provide as feedback the predicted second set of gene-level endophenotype profiles to the second trained machine-learning model.
75A. The system of embodiment 74A, wherein the instructions to provide as feedback the predicted second set of gene-level endophenotype profiles to the second trained machine-learning model further comprise instructions to refine the prediction of the second set of gene-level endophenotype profiles in accordance with a predetermined evaluation metric.
76A. The system of any one of embodiments 72A-75A, wherein the first trained machine-learning model comprises one or more sequence encoder models including language-based models adapted from natural language processing (NLP) and one or more variant effect predictor models including classification or regression models.
77A. The system of any one of embodiments 72A-76A, wherein the instructions further comprise instructions to: train the first trained machine-learning model by: pre-training a randomly-initialized language model utilizing a self-supervised prediction of one or more gene regulatory sequences extracted from a wide variety of species; and fine-tuning the pre-trained language model utilizing a self-supervised prediction of a plurality of gene regulatory sequences extracted from a targeted species.
78A. The system of embodiment 77A, wherein training the first machine-learning model further comprises: training a regression or classification model with input features generated by the fine-tuned language model to generate effect predictions corresponding to a plurality of candidate endophenotypes of interest.
79A. The system of embodiment 78A, wherein the instructions further comprise instructions to utilize the regression or classification model to predict a particular endophenotype measurement observed from one or more cell-based assays or one or more plant-based assays.
80A. The system of embodiment 79A, wherein the instructions further comprise instructions to: obtain the particular endophenotype measurement from the one or more cell-based assays or one or more plant-based assays; and train the regression or classification model by: computing a loss value based on a comparison of the effect predictions and the endophenotype measurement; and training the regression or classification model based on a backpropagation of the computed loss value.
81A. The system of any one of embodiments 72A-80A, wherein the second trained machine-learning model comprises one or more graph neural networks (GNNs).
82A. The system of embodiment 81A, wherein the instructions further comprise instructions to: train the second machine-learning model by: aggregating a dataset of endophenotype profiles of various genotypes corresponding to a target organism or pathway; and selecting one or more random pairs of genotypes from the dataset of endophenotype profiles of genotypes for which each pair represents an unmodified and modified organism respectively.
83A. The system of claim 82A, wherein training the second machine-learning model further comprises: initializing the one or more GNNs by randomly partitioning nodes of graphs comprising the one or more GNNs into a first set of nodes corresponding to a first genotype of the one or more random pairs of genotypes and a second set of nodes corresponding to a second genotype of the one or more random pairs of genotypes.
84A. The system of claim 83A, wherein training the second machine-learning model further comprises; and training the one or more GNNs to predict the endophenotypes corresponding to first set of nodes for the second genotype given both the endophenotypes corresponding to the first set of nodes for the first genotype and the endophenotypes corresponding to the second set of nodes for the second genotype.
85A. The system of any one of embodiments 72A-82A, wherein the second set of gene-level endophenotype profiles is predicted for a modified genotype of one or more plant seeds.
86A. The system of any one of embodiments 72A-85A, wherein the first trained machine-learning model and the second trained machine-learning model were trained utilizing data representing information related to RNAseq, microarrays, ribosome profiling, single cell RNASeq, and/or proteome abundance.
87A. The system of any one of embodiments 72A-86A, wherein the first set of gene-level endophenotype profiles comprises a tissue-specific gene endophenotype, a temporally-controlled gene endophenotype, or a change in gene endophenotype in response to a stimulus.
88A. A non-transitory computer-readable medium comprising instructions that, when executed by one or more processors of one or more computing devices, cause the one or more processors to: input a plurality of gene regulatory sequences to a first trained machine-learning model, the plurality of gene regulatory sequences including one or more mutated gene regulatory sequences; utilize the first trained machine-learning model to generate a first set of gene-level endophenotype profiles based on the plurality of gene regulatory sequences, including the cis regulatory effects of the one or more mutated gene regulatory sequences; input the first set of gene-level endophenotype profiles to a second trained machine-learning model; and utilize the second trained machine-learning model to generate a second set of gene-level endophenotype profiles based on the first set of gene-level endophenotype profiles, wherein generating the second set of gene-level endophenotype profiles comprises predicting one or more updated gene-level endophenotype profiles based on the plurality of gene regulatory sequences including the trans regulatory effects of the one or more mutated gene regulatory sequences.
1B. A method of regulating two or more genes in a plant, the method comprising, a) by one or more computing devices: i) obtaining one or more endophenotype profiles corresponding to a genotype; ii) partitioning the one or more endophenotype profiles into a first set of endophenotypes and a second set of endophenotypes; iii) receiving an input to modify the first set of endophenotypes to a desired level; and iv) inputting the modified first set of endophenotypes and unmodified second set of endophenotypes into a trained machine-learning model to obtain a prediction of an updated second set of endophenotypes, wherein the updated second set of endophenotypes represents an updated version of the second set of endophenotypes after interacting with the modified first set of endophenotypes; and b) modifying an endophenotype level of one or more predicted interacting partner genes by modifying the first set of endophenotypes.
2B. The method of embodiment 1B, wherein modifying an endophenotype level of one or more predicted interacting partner genes by modifying the first set of endophenotypes comprises introducing the one or more modified genotypes into the plant.
3B. The method of any one of embodiments 1B-2B, further comprising after step iv): v) comparing the prediction of the updated second set of endophenotypes to a desired level.
4B. The method of embodiment 3B, further comprising: vi) if the prediction of the updated second set of endophenotypes does not reach a desired level, return to step iii), receiving an input comprising an altered set of one or more modified genotypes predicted to modify the first set of endophenotypes to the desired level.
5B. A method for predicting endophenotypes of interacting partner genes, the method comprising, by one or more computing devices: obtaining one or more endophenotype profiles corresponding to a genotype; partitioning the one or more endophenotype profiles into a first set of endophenotypes and a second set of endophenotypes; receiving an input to modify the first set of endophenotypes to a desired level, wherein the input comprises one or more modified genotypes predicted to modify the first set of endophenotypes to the desired level; and inputting the modified first set of endophenotypes and unmodified second set of endophenotypes into a trained machine-learning model to obtain a prediction of an updated second set of endophenotypes, wherein the updated second set of endophenotypes represents an updated version of the second set of endophenotypes after interacting with the modified subset of the first set of endophenotypes.
6B. The method of any one of embodiments 1B-5B, wherein the one or more computing devices are associated with a genome editing platform, and wherein the genome editing platform is configured to assess updates to the second set of endophenotypes as a result of trans regulatory effects.
7B. The method of any one of embodiments 1B-6B, wherein obtaining the one or more endophenotype profiles comprises obtaining one or more endophenotype profiles corresponding to a target genotype.
8B. The method of any one of embodiments 1B-7B, further comprising providing as feedback the updated second set of endophenotypes to the trained machine-learning model in place of the original second set of endophenotypes in order to refine the prediction of the updated second set of endophenotype levels in accordance with a predetermined evaluation metric.
9B. The method of any one of embodiments 1B-8B, wherein the trained machine-learning model comprises one or more graph neural networks (GNNs).
10B. The method of embodiment 9B, wherein inputting the first set of endophenotypes into the trained machine-learning model comprises inputting node representation vectors to a graph neural network (GNN).
11B. The method of any one of embodiments 9B-10B, wherein nodes of graphs comprising the one or more GNNs represent genes associated with the target organism or pathway.
12B. The method of any one of embodiments 9B-11B, wherein edges of the graphs comprising the one or more GNNs represent predictions of interactions in trans between genes associated with the target organism or pathway.
13B. The method of any one of embodiments 9B-12B, wherein the graphs comprising the one or more GNNs further comprise one or more known gene co-expression relationships, one or more known protein-to-protein interactions, one or more gene ontology relationships, or a combination thereof.
14B. The method of any one of embodiments 1B-13B, further comprising: training the machine-learning model by: aggregating a dataset of endophenotype profiles corresponding to various genotypes comprising a target organism or pathway; and selecting one or more random pairs of genotypes from the dataset of endophenotype profiles of genotypes for which each pair represents an unmodified and modified organism respectively.
15B. The method of embodiment 14B, wherein training the machine-learning model further comprises: initializing one or more graph neural networks (GNNs) by randomly partitioning nodes of graphs comprising the one or more GNNs into a first set of nodes corresponding to a first genotype of the one or more random pairs of genotypes and a second set of nodes corresponding to a second genotype of the one or more random pairs of genotypes.
16B. The method of embodiment 15B, wherein training the machine-learning model further comprises: training the one or more GNNs to predict the endophenotypes corresponding to the first set of nodes for the second genotype given both the endophenotypes corresponding to the first set of nodes for the first genotype and the endophenotypes corresponding to the second set of nodes for the second genotype.
17B. The method of any one of embodiments 1B-16B, wherein obtaining the one or more endophenotype profiles comprises accessing an aggregate of a plurality of gene interaction data to be utilized to construct one or more gene regulatory network graphs.
18B. The method of embodiment 17B, wherein the plurality of gene interaction data comprises one or more known gene co-expression relationships, one or more known protein-to-protein interactions, one or more gene ontology relationships, or a combination thereof.
19B. The method of any one of embodiments 1B-18B, wherein the one or more predicted interacting partner genes in the genome comprises one or more predicted interacting partner genes in a modified genotype of one or more plant seeds.
20B. The method of any one of embodiments 1B-19B, wherein the machine-learning model was trained utilizing data representing information related to RNAseq, microarrays, ribosome profiling, single cell RNASeq, and/or proteome abundance.
21B. The method of any one of embodiments 1B-20B, wherein the endophenotype comprises a tissue-specific gene endophenotype, a temporally-controlled gene endophenotype, or a change in gene endophenotype in response to a stimulus.
22B. The method of any one of embodiments 1B-21B, further comprising providing genome editing molecules to a plant to introduce the one or more modified genotypes to the plant based on the one or more predicted endophenotype profiles.
23B. The method of embodiment 22B, wherein the genome editing molecules comprise an endonuclease and one or more guide RNAs.
24B. The method of any one of embodiments 22B-23B, wherein the genome editing molecules further comprise a donor template nucleic acid comprising the sequence of the one or more modified genotypes.
25B. A plant comprising predicted endophenotype profiles generated by the method of any one of embodiments 1B-4B or 22B-24B.
26B. A system including one or more computing devices, comprising: one or more non-transitory computer-readable storage media including instructions; and one or more processors coupled to the one or more storage media, the one or more processors configured to execute the instructions to: obtain one or more endophenotype profiles corresponding to a genotype; partition the one or more endophenotype profiles into a first set of endophenotypes and a second set of endophenotypes; receive an input to modify the first set of endophenotypes to a desired level; and input the modified first set of endophenotypes and unmodified second set of endophenotypes into a trained machine-learning model to obtain a prediction of an updated second set of endophenotypes, wherein the updated second set of endophenotypes represents an updated version of the second set of endophenotypes after interacting with the modified subset of the first set of endophenotypes.
27B. The system of embodiment 26B, wherein the one or more computing devices are associated with a genome editing platform, and wherein the genome editing platform is configured to assess updates to the second set of endophenotypes as a result of trans regulatory effects.
28B. The system of any one of embodiments 26B-27B, wherein the instructions to obtain the one or more endophenotype profiles further comprise instructions to obtain one or more endophenotype profiles corresponding to a target genotype.
29B. The system of any one of embodiments 26B-28B, wherein the instructions further comprise instructions to provide as feedback the updated second set of endophenotypes to the trained machine-learning model in place of the original second set of endophenotypes in order to refine the prediction of the updated second set of endophenotype levels in accordance with a predetermined evaluation metric.
30B. The system of any one of embodiments 26B-29B, wherein the trained machine-learning model comprises one or more graph neural networks (GNNs).
31B. The system of embodiment 30B, wherein the instructions to input the first set of endophenotypes into the trained machine-learning model further comprise instructions to input node representation vectors to a graph neural network (GNN).
32B. The system of any one of embodiments 30B-31B, wherein nodes of graphs comprising the one or more GNNs represent genes associated with the target organism or pathway.
33B. The system of any one of embodiments 30B-32B, wherein edges of the graphs comprising the one or more GNNs represent predictions of interactions in trans between genes associated with the target organism or pathway.
34B. The system of any one of embodiments 30B-33B, wherein the graphs comprising the one or more GNNs further comprise one or more known gene co-expression relationships, one or more known protein-to-protein interactions, one or more gene ontology relationships, or a combination thereof.
35B. The system of any one of embodiments 26B-34B, wherein the instructions further comprise instructions to: train the machine-learning model by: aggregating a dataset of endophenotype profiles corresponding to various genotypes comprising a target organism or pathway; and selecting one or more random pairs of genotypes from the dataset of endophenotype profiles of genotypes for which each pair represents an unmodified and modified organism respectively.
36B. The system of embodiment 35B, wherein the instructions to train the machine-learning model further comprise instructions to: initialize one or more graph neural networks (GNNs) by randomly partitioning nodes of graphs comprising the one or more GNNs into a first set of nodes corresponding to a first genotype of the one or more random pairs of genotypes and a second set of nodes corresponding to a second genotype of the one or more random pairs of genotypes.
37B. The system of embodiment 36B, wherein the instructions to train the machine-learning model further comprise instructions to: train the one or more GNNs to predict the endophenotypes corresponding to the first set of nodes for the second genotype given both the endophenotypes corresponding to the first set of nodes for the first genotype and the endophenotypes corresponding to the second set of nodes for the second genotype.
38B. The system of any one of embodiments 26B-37B, wherein the instructions to obtain the one or more endophenotype profiles further comprise instructions to access an aggregate of a plurality of gene interaction data to be utilized to construct one or more gene regulatory network graphs.
39B. The system of embodiment 38B, wherein the plurality of gene interaction data comprises one or more known gene co-expression relationships, one or more known protein-to-protein interactions, one or more gene ontology relationships, or a combination thereof.
40B. The system of any one of embodiments 26B-39B, wherein the one or more predicted interacting partner genes in the genome comprises one or more predicted interacting partner genes in a modified genotype of one or more plant seeds.
41B. The system of any one of embodiments 26B-40B, wherein the machine-learning model was trained utilizing data representing information related to RNAseq, microarrays, ribosome profiling, single cell RNASeq, and/or proteome abundance.
42B. The system of any one of embodiments 26B-41B, wherein the endophenotype comprises a tissue-specific gene endophenotype, a temporally-controlled gene endophenotype, or a change in gene endophenotype in response to a stimulus.
43B. The system of any one of embodiments 27B-42B, wherein the genome editing platform is further configured to introduce the one or more modified genotypes to a plant based on the one or more predicted endophenotype profiles.
44B. A non-transitory computer-readable medium comprising instructions that, when executed by one or more processors of one or more computing devices, cause the one or more processors to: obtain one or more endophenotype profiles corresponding to a genotype; partition the one or more endophenotype profiles into a first set of endophenotypes and a second set of endophenotypes; receive an input to modify the first set of endophenotypes to a desired level; and input the modified first set of endophenotypes and unmodified second set of endophenotypes into a trained machine-learning model to obtain a prediction of an updated second set of endophenotypes, wherein the updated second set of endophenotypes represents an updated version of the second set of endophenotypes after interacting with the modified subset of the first set of endophenotypes.
45B. The non-transitory computer-readable medium of embodiment 44B, wherein the one or more computing devices are associated with a genome editing platform, and wherein the genome editing platform is configured to predict updates to the second set of endophenotypes as a result of trans regulatory effects.
46B. The non-transitory computer-readable medium of any one of embodiments 44B-45B, wherein the instructions to obtain the one or more endophenotype profiles further comprise instructions to obtain one or more endophenotype profiles corresponding to a target genotype.
47B. The non-transitory computer-readable medium of any one of embodiments 44B-46B, wherein the instructions further comprise instructions to provide as feedback the updated second set of endophenotypes to the trained machine-learning model in place of the original second set of endophenotypes in order to refine the prediction of the updated second set of endophenotype levels in accordance with a predetermined evaluation metric.
48B. The non-transitory computer-readable medium of any one of embodiments 44B-47B, wherein the trained machine-learning model comprises one or more graph neural networks (GNNs).
49B. The non-transitory computer-readable medium of embodiment 48B, wherein the instructions to input the first set of endophenotypes into the trained machine-learning model further comprise instructions to input node representation vectors to a graph neural network (GNN).
50B. The non-transitory computer-readable medium of any one of embodiments 48B-49B, wherein nodes of graphs comprising the one or more GNNs represent genes associated with the target organism or pathway.
51B. The non-transitory computer-readable medium of any one of embodiments 48B-50B, wherein edges of the graphs comprising the one or more GNNs represent predictions of interactions in trans between genes associated with the target organism or pathway.
52B. The non-transitory computer-readable medium of any one of embodiments 48B-51B, wherein the graphs comprising the one or more GNNs further comprise one or more known gene co-expression relationships, one or more known protein-to-protein interactions, one or more gene ontology relationships, or a combination thereof.
53B. The non-transitory computer-readable medium of any one of embodiments 44B-52B, wherein the instructions further comprise instructions to: train the machine-learning model by: aggregating a dataset of endophenotype profiles corresponding to various genotypes comprising a target organism or pathway; and selecting one or more random pairs of genotypes from the dataset of endophenotype profiles of genotypes for which each pair represents an unmodified and modified organism respectively.
54B. The non-transitory computer-readable medium of embodiment 53B, wherein the instructions to train the machine-learning model further comprise instructions to: initialize one or more graph neural networks (GNNs) by randomly partitioning nodes of graphs comprising the one or more GNNs into a first set of nodes corresponding to a first genotype of the one or more random pairs of genotypes and a second set of nodes corresponding to a second genotype of the one or more random pairs of genotypes.
55B. The non-transitory computer-readable medium of embodiment 54B, wherein the instructions to train the machine-learning model further comprise instructions to: train the one or more GNNs to predict the endophenotypes corresponding to the first set of nodes for the second genotype given both the endophenotypes corresponding to the first set of nodes for the first genotype and the endophenotypes corresponding to the second set of nodes for the second genotype.
56B. The non-transitory computer-readable medium of any one of embodiments 44B-53B, wherein the instructions to obtain the one or more endophenotype profiles further comprise instructions to access an aggregate of a plurality of gene interaction data to be utilized to construct one or more gene regulatory network graphs.
57B. The non-transitory computer-readable medium of embodiment 56B, wherein the plurality of gene interaction data comprises one or more known gene co-expression relationships, one or more known protein-to-protein interactions, one or more gene ontology relationships, or a combination thereof.
58B. The non-transitory computer-readable medium of any one of embodiments 44B-57B, wherein the one or more predicted interacting partner genes in the genome comprises one or more predicted interacting partner genes in a modified genotype of one or more plant seeds.
59B. The non-transitory computer-readable medium of any one of embodiments 44B-58B, wherein the machine-learning model was trained utilizing data representing information related to RNAseq, microarrays, ribosome profiling, single cell RNASeq, and/or proteome abundance.
60B. The non-transitory computer-readable medium of any one of embodiments 44B-59B, wherein the endophenotype comprises a tissue-specific gene endophenotype, a temporally-controlled gene endophenotype, or a change in gene endophenotype in response to a stimulus.
61B. The non-transitory computer-readable medium of any one of embodiments 45B-60B, wherein the genome editing platform is further configured to introduce the one or more modified genotypes to a plant based on the one or more predicted endophenotype profiles.
62B. The method of any one of embodiments 1B-24B, wherein receiving an input to modify the first set of endophenotypes to a desired level comprises receiving one or more modified genotypes predicted to modify the first set of endophenotypes to the desired level.
63B. The system of any one of embodiments 26B-43B, wherein the instructions to receive an input to modify the first set of endophenotypes to a desired level further comprise receiving one or more modified genotypes predicted to modify the first set of endophenotypes to the desired level.
64B. The non-transitory computer-readable medium of any one of embodiments 44B-61B, wherein the instructions to receive an input to modify the first set of endophenotypes to a desired level further comprise receiving one or more modified genotypes predicted to modify the first set of endophenotypes to the desired level.

1. A method of modifying an endophenotype in a plant, the method comprising, by one or more computing devices:
obtaining a plurality of gene regulatory sequences;
inputting the plurality of gene regulatory sequences into a machine-learning model trained to obtain a plurality of effect predictions corresponding to a plurality of endophenotypes;
selecting one or more desired endophenotypes based on the plurality of endophenotypes;
selecting a gene regulatory sequence in accordance with the one or more desired endophenotypes, and
introducing the selected gene regulatory sequence into the plant, thereby modifying the endophenotype of the plant.

2. A method for generating a gene regulatory sequence with a desired endophenotype profile, the method comprising, by one or more computing devices:
obtaining a plurality of gene regulatory sequences;
inputting the plurality of gene regulatory sequences into a machine-learning model trained to obtain a plurality of effect predictions corresponding to a plurality of endophenotypes;
selecting one or more desired endophenotypes based on the plurality of endophenotypes; and
selecting a gene regulatory sequence in accordance with the one or more desired endophenotypes.

3. The method of claim 1, wherein selecting the gene regulatory sequence comprises selecting a gene regulatory sequence in accordance with a desired endophenotype level.

4. The method of claim 3, wherein the desired endophenotype level comprises a desired messenger RNA (mRNA) expression level.

5. The method of claim 1, wherein the one or more computing devices are associated with a genome editing platform, the genome editing platform configured to generate the gene regulatory sequence with the desired endophenotype profile.

6. The method of claim 1, wherein obtaining the plurality of gene regulatory sequences comprises: obtaining a plurality of gene promoter regulatory sequences, a plurality of gene terminator regulatory sequences, a plurality of gene enhancer regulatory sequences, a plurality of gene repressor regulatory sequences, a plurality of transcription factor binding sites, and/or a plurality of synthetic gene regulatory sequences.

7. The method of claim 1, wherein the machine-learning model comprises one or more sequence encoder models.

8. The method of claim 1, wherein the machine-learning model is trained by:
pre-training a randomly-initialized sequence encoder model utilizing a self-supervised prediction of the one or more gene regulatory sequences; and
fine-tuning the pre-trained sequence encoder model utilizing a self-supervised prediction of a plurality of gene regulatory sequences extracted from a targeted taxonomic unit.

9. The method of claim 8, wherein the machine-learning model is trained further by:
utilizing a variant effect predictor model with inputs generated by the sequence encoder model to: 1) further fine-tune the weights of the sequence encoder model and 2) generate effect predictions corresponding to a plurality of candidate endophenotypes of interest.

10. The method of claim 9, wherein the machine-learning model is trained further by:
computing a loss value based on a comparison of the effect predictions and an endophenotype measurement; and
training the variant effect predictor model based on a backpropagation of the computed loss value.

11. The method of claim 10, further comprising utilizing the variant effect predictor model to predict a particular endophenotype measurement observed from one or more cell-based assays or one or more plant-based assays.

12. The method of claim 1, wherein the machine-learning model comprises one or more sequence space-sampling algorithms.

13. The method of claim 12, further comprising:
subsequent to obtaining the plurality of gene regulatory sequences:
inputting a plurality of seed gene regulatory sequences into the one or more sequence space-sampling algorithms; and
obtaining the plurality of effect predictions by: 1) computationally sampling the space of gene regulatory sequences, and 2) inputting the plurality of sampled gene regulatory sequences into the one or more trained machine-learning models to obtain the plurality of effect predictions corresponding to the plurality of endophenotypes.

14. The method of claim 13, wherein the one or more sequence space-sampling algorithms comprise one or more generative adversarial networks (GANs), one or more variational autoencoders (VAEs), or one or more Markov chain Monte Carlo (MCMC) sampling algorithms.

15. The method of claim 13, wherein obtaining the plurality of effect predictions corresponding to the plurality of endophenotypes comprises iteratively providing as feedback a plurality of sampled gene regulatory sequences as seed sequences for the one or more sequence space-sampling algorithms until the one or more desired endophenotypes are produced.

16. The method of claim 1, wherein the selected gene regulatory sequence is operably linked to an exogenous or endogenous transcript, and is provided in a vector for expressing the exogenous or endogenous transcript.

17. The method of claim 1, further comprising generating a donor template nucleic acid comprising the gene regulatory sequence or a portion thereof.

18. The method of claim 1, further comprising generating one or more guide RNAs (gRNAs) targeting a genomic location to promote introduction of the gene regulatory sequence.

19. The method of claim 17, wherein the guide RNA and/or donor template nucleic acid is configured to introduce a selected modified gene regulatory sequence into one or more plants.

20. The method of claim 1, wherein the machine-learning model was trained utilizing data representing information related to RNAseq, microarrays, ribosome profiling, single cell RNASeq, and/or proteome abundance.

21. The method of claim 1, wherein the one or more desired endophenotypes comprises a tissue-specific gene endophenotype, a temporally-controlled gene endophenotype, or a change in gene endophenotype in response to a stimulus.

22. (canceled)

23. A plant comprising a modified gene regulatory sequence generated by the method of claim 1.

24. A system including one or more computing devices, comprising:
one or more non-transitory computer-readable storage media including instructions; and
one or more processors coupled to the one or more storage media, the one or more processors configured to execute the instructions to:
obtain a plurality of gene regulatory sequences;
input the plurality of gene regulatory sequences into a machine-learning model trained to obtain a plurality of effect predictions corresponding to a plurality of endophenotypes;
select one or more desired endophenotypes based on the plurality of endophenotypes; and
select a gene regulatory sequence in accordance with the one or more desired endophenotypes.

25.-42. (canceled)

43. A non-transitory computer-readable medium comprising instructions that, when executed by one or more processors of one or more computing devices, cause the one or more processors to:
obtain a plurality of gene regulatory sequences;
input the plurality of gene regulatory sequences into a machine-learning model trained to obtain a plurality of effect predictions corresponding to a plurality of endophenotypes;
select one or more desired endophenotypes based on the plurality of endophenotypes; and
select a gene regulatory sequence in accordance with the one or more desired endophenotypes.

44.-53. (canceled)

54. A method for predicting the effect of a mutated gene regulatory sequence the method comprising, by one or more computing devices:
inputting a plurality of gene regulatory sequences to a first trained machine-learning model, the plurality of gene regulatory sequences comprising one or more mutated gene regulatory sequences;
utilizing the first trained machine-learning model to generate a first set of gene-level endophenotype profiles based on the plurality of gene regulatory sequences, comprising cis regulatory effects of the one or more mutated gene regulatory sequences;
inputting the first set of gene-level endophenotype profiles to a second trained machine-learning model; and
utilizing the second trained machine-learning model to generate a second set of gene-level endophenotype profiles based on the first set of gene-level endophenotype profiles, wherein generating the second set of gene-level endophenotype profiles comprises predicting one or more updated gene-level endophenotype profiles based on the plurality of gene regulatory sequences including the trans regulatory effects of the one or more mutated gene regulatory sequences.

55.-69. (canceled)

70. The method of claim 54, further comprising introducing a mutated gene regulatory sequence to a plant based on the one or more predicted gene-level endophenotype profiles.

71. A plant comprising a mutated gene regulatory sequence and/or predicted gene-level endophenotype profiles generated by the method of claim 70.

72.-88. (canceled)
